(19) United States
(12) Patent Application Publication
(10) Pub. No.: US 2011/0195910 A1
(43) Pub. Date: Aug. 11, 2011

(54) USE OF TOCOTRIENOL COMPOSITION FOR THE PREVENTION OF CANCER

(75) Inventors: Ming Tat Ling, Brisbane (AU); Wei Ney Yap, Singapore (SG); Yong Chuan Wong, Hong Kong (CN); Yee Leng Daniel Yap, Singapore (SG)

(73) Assignee: DAVOS LIFE SCIENCE PTE. LTD., Singapore (SG)

(21) Appl. No.: 12/599,486

(22) PCT Filed: Oct. 20, 2009
(86) PCT No.: PCT/SG2009/000390

§ 371 Date: Nov. 9, 2009

Related U.S. Application Data
(60) Provisional application No. 61/107,842, filed on Oct. 23, 2008.

(51) Int. Cl. A61K 31/352; A61K 38/00; A61K 31/4164; A61P 35/00

(52) U.S. Cl. 514/19.3; 514/456; 514/398

(57) ABSTRACT

The present invention is directed to a method of preventing cancer or preventing the recurrence of cancer after undergoing a cancer treatment by administering a composition comprising at least one of γ-tocotrienol or δ-tocotrienol, wherein the cancer is selected from the group consisting of melanoma, prostate cancer, prostate intraepithelial neoplasia, colon cancer, liver cancer, bladder cancer, breast cancer and lung cancer. The present invention is further directed to a composition comprising at least one of γ-tocotrienol or δ-tocotrienol and Docetaxel and/or Dacarbazine, and to a method of inhibiting or arresting or reversing of cancer by administering a composition comprising at least one of γ-tocotrienol or δ-tocotrienol together with Docetaxel and/or Dacarbazine. The present invention is also directed to methods of manufacturing those compositions.

### CROSS-REFERENCE TO RELATED APPLICATIONS

This application claims the benefit of priority of U.S. provisional application No. 61/107,842, filed Oct. 23, 2008, the contents of it being hereby incorporated by reference in its entirety for all purposes.

### FIELD OF THE INVENTION

The present invention is directed to the field of molecular biology and biochemistry, in particular the field of biochemistry and molecular biology of cancer.

### BACKGROUND OF THE INVENTION

Cancer or more precisely malignant neoplasm is a class of diseases in which a group of cells display uncontrolled growth (division beyond the normal limits), invasion (intrusion on and destruction of adjacent tissues), and sometimes metastasis (spread to other locations in the body via lymph or blood).

The progression, or lack thereof, of a given cancer is highly variable and depends on the type of neoplasm and the response to treatment. Treatment modalities include surgery, chemotherapy, radiation therapy, hormonal manipulation, and immunotherapy. In general, each type of cancer is treated very specifically, and often a combination of the various modalities is used, for example, surgery preceded or followed by radiation therapy. The response to treatment depends on the type of tumor, its size, and whether it has spread.

Most of the known methods of treating cancer have severe side effects on the patient. Therefore, it is an object of the present invention to explore further ways of treating cancer.

### SUMMARY OF THE INVENTION

In a first aspect, the present invention refers to a method of preventing cancer or preventing the recurrence of cancer after undergoing a cancer treatment by administering a composition comprising or consisting of at least one of γ-tocotrienol or δ-tocotrienol, wherein the cancer is selected from the group consisting of melanoma, prostate cancer, colon cancer, liver cancer, bladder cancer, breast cancer and lung cancer.

In a further aspect, the present invention refers to a composition comprising or consisting of at least one of γ-tocotrienol or δ-tocotrienol and (2R,3S)—N-carboxy-3-phenylisoserine, N-tert-butylester, 13-ester with 5,20-epoxy-1,2,4,7,10,13-hexahydroxytax-11-en-9-one-4-acetate-2-benzoate, trihydrate (Docetaxel) and/or (5Z)-5-(dimethylaminohydrazinylidene)imidazole-4-carboxamide (Dacarbazine).

In still a further aspect, the present invention refers to a method of inhibiting or reversing of cancer by administering a composition comprising or consisting of at least one of γ-tocotrienol or δ-tocotrienol together with (2R,3S)—N-carboxy-N-tert-butylester-3-phenylisoserine, 13-ester with 5,20-epoxy-1,2,4,7,10,13-hexahydroxytax-11-en-9-one-4-acetate-2-benzoate, trihydrate (Docetaxel) and/or (5Z)-5-(dimethylaminohydrazinylidene)imidazole-4-carboxamide (Dacarbazine).

In another aspect, the present invention refers to the use of a composition comprising at least one of γ-tocotrienol or δ-tocotrienol for the manufacture of a medicament for preventing cancer in an animal body or preventing the recurrence of cancer in an animal body after undergoing a cancer treatment, wherein the cancer is selected from the group consisting of melanoma, prostate cancer, colon cancer, liver cancer, bladder cancer, breast cancer and lung cancer.

In still another aspect, the present invention refers to the use of a composition comprising at least one of γ-tocotrienol or δ-tocotrienol together with (2R,3S)—N-carboxy-3-phenylisoserine, N-tert-butylester, 13-ester with 5,20-epoxy-1,2,4,7,10,13-hexahydroxytax-11-en-9-one-4-acetate-2-benzoate, trihydrate (Docetaxel) or (5Z)-5-(dimethylaminohydrazinylidene)imidazole-4-carboxamide (Dacarbazine) for the manufacture of a medicament for the treatment of cancer.

In a further aspect, the present invention refers to a method of manufacturing a composition comprising or consisting of at least one of γ-tocotrienol or δ-tocotrienol and (2R,3S)—N-carboxy-3-phenylisoserine, N-tert-butylester, 13-ester with 5,20-epoxy-1,2,4,7,10,13-hexahydroxytax-11-en-9-one-4-acetate-2-benzoate, trihydrate (Docetaxel) and/or (5Z)-5-(dimethylaminohydrazinylidene)imidazole-4-carboxamide (Dacarbazine), comprising mixing at least one γ-tocotrienol or δ-tocotrienol with (2R,3S)—N-carboxy-3-phenylisoserine, N-tert-butylester, 13-ester with 5,20-epoxy-1,2,4,7,10,13-hexahydroxytax-11-en-9-one-4-acetate-2-benzoate, trihydrate (Docetaxel) and/or (5Z)-5-(dimethylaminohydrazinylidene)imidazole-4-carboxamide (Dacarbazine).

### BRIEF DESCRIPTION OF THE DRAWINGS

The invention will be better understood with reference to the detailed description when considered in conjunction with the non-limiting examples and the accompanying drawings, in which:

FIG. 1 illustrates the results of experiments demonstrating that gamma-T3 down-regulates prostate cancer stem cell markers in PC-3 cells. (A) Western blotting of prostate cancer stem cell markers CD44 and CD133 after γ-T3 treatment. Note that γ-T3 significantly down-regulates both stem cell markers in a dose and time dependent manner. (B) Flow cytometry analysis of CD44⁺ population in PC-3 cells after 5 μg/ml of γ-T3 treatment for 24 hrs. CD44⁺ population after γT3-treatment, as indicated by arrow, was reduced compared to untreated control (shaded peak). (C) mRNA levels of CD44 and CD133 after γ-T3 treatment. Note that markers were decreased after 48 and 72 hrs of treatment. Samples were normalized by GAPDH. (D) Viability of PC-3 cells after treatment with 2.5 and 5 μg/ml of γ-T3 for 24, 48 and 72 hrs was examined by MTT assay. Each experiment was repeated for at least three times. Results are presented as mean±standard deviation (s.d.) (E) Western blotting result of apoptotic markers in γ-T3 treated PC-3 cells. Note that no cleaved form of PARP, caspase-3, -7, -9 were detected, indicating no induction of apoptosis by γ-T3 treatment.

FIG. 2 illustrates the results of experiments demonstrating that gamma-T3 suppresses stem cell property of PC-3 cells. (A) Spheroid formation assay was performed with cells that treated with γ-T3 or vehicle. Two hundred PC-3 cells were seeded on polyHEMA-coated 12-well plates and treated with either γ-T3 or vehicle for 14 days. The number of prostaspheres formed was counted and the result was presented as mean±s.d. Note that γ-T3 treatment efficiently suppresses the spheroid formation ability of PC-3 cells. (B) Image of the prostaspheres was captured under microscope. Note that no prostaspheres can be found in γ-T3 treated group.

FIG. 3 illustrates the results of experiments showing that gamma-T3 suppresses cancer stem-like cells also in other cancer cell lines. (A) Western blotting of CD44 in vehicle and γ-T3 treated DU145 and MGH-U1 cells. CD44 expressions of both cell lines were down-regulated after low dose γ-T3 treatment. (N.B. 5 μg/ml of γ-T3 is equivalent to 12.176 μM). (B&C) MTT assay showing the viability of DU145 and MOI-UI (Bladder cancer) cells after treatment with different dosages of γ-T3 for 24 and 48 hrs. (D&E) Spheroid formation assay was performed with cells that treated with γ-T3 or vehicle. Note that γ-T3 treatment efficiently suppresses the spheroid formation ability of both cell lines. Images of the spheroids were captured under microscope. Note that no spheroid can be found in γ-T3 treated groups.

FIG. 4 illustrates the results of experiments showing that gamma-T3 significantly reduces the tumorigenicity of PC-3 cells in vivo. (A) Bioluminescent image of SCID mice that orthotopically injected with PC-3-luc cells for 2 weeks. SCID mice at upper row were injected with vehicle treated PC-3-luc cells where as mice in bottom row were injected with γ-T3 treated PC-3-luc cells. Note that 3 mice from γ-T3 group did not show detectable tumor. (B) The percentage of mice developing detectable tumors at week 2. Note that over half of the mice in γ-T3 group did not form detectable tumor whereas 100% tumor formation were found in control group. n=16.

FIG. 5 illustrates the results of experiments demonstrating the effect of γ-T3 on targeting cancer stem cell-enriched prostaspheres. (A) CSC enriched prostaspheres were formed by maintaining DU145 cells in non-adherent culture supplemented with serum replacement medium for 14 days. The prostaspheres were then treated with either vehicle, γ-T3 (10, 20 μg/ml) or Docetaxel (Doc, 40 ng/ml) for 48 hrs. Spheroids were counted under microscope before and after treatment. Results were presented as mean % change in spheroid number to control±s.d. Note that spheroids were highly sensitive to γ-T3 treatment but resistant to high dose of Docetaxel. (B) Images of prostaspheres after 48 hrs treatment with vehicle, 40 ng/ml of Docetaxel and 10 μg/ml of γ-T3. Gamma-T3 treated spheroids were found to be dissociated.

FIG. 6(A) shows that γT3 was not determined to affect mTOR and β-catenin, but suppresses Akt signalling pathway. Activation of AKT signalling pathway is highly correlated with human prostate cancer and transgenic animals that express a constitutively active form of AKT develop prostatic intraepithelial neoplasia. (B) γT3 enhanced OCT3/4 and Nestin mRNA expression, the key regulators for pluripotent stem cell phenotype.

FIG. 7 illustrates a specific embodiment of one aspect of the present invention in which a composition comprising at least one of γ-tocotrienol or δ-tocotrienol is used to prevent cancer (in the embodiment illustrated in FIG. 7 prostate cancer) before it occurs (third pathway from the top) and after it has been treated with conventional cancer therapy (second pathway from the top). The first pathway illustrates the normal therapy in which a solid prostate cancer tumor comprising prostate cancer stem cells (PCSC) is treated with a conventional cancer therapy, such as chemotherapy or with a chemotherapeutic drug, such as docetaxel. Since those therapies do not affect the PCSCs the tumor can redevelop based on the PCSCs. As has been demonstrated in experiments referred to herein, when a composition comprising at least one of γ-tocotrienol or δ-tocotrienol is administered to an animal body the development of a solid prostate cancer tumor can be prevented (third pathway). Furthermore, application of a composition comprising at least one of γ-tocotrienol or δ-tocotrienol after a tumor treatment can prevent that PCSCs initiate cancer cell renewal, i.e. the composition claimed herein prevents the recurrence of cancer.

FIG. 8 shows that γ-T3, and δ-T3 and γ-T3-comprising composition prevent the formation of prostate intraepithelial neoplasia (PIN), the most likely precursor of prostate cancer development. The prostate cancer mouse models used were previously published (Gabril, M. Y., Duan, W., et al., Molecular Therapy (2005), vol. 11, no. 3, p. 348; Greenberg et al., Proc Natl Acad Sci USA (1995), vol. 92, pp. 3439-3443; Duan, W., Gabril, M. Y., et al., Oncogene (2005) 24, 1510-1524; Wang S, Gao J, et al., Cancer Cell., 2003, vol. 4, no. 3, pp. 209-21; Gabril, M. Y., Onita, T., et al., Gene Ther., 2002, vol. 9, no. 23, pp. 1589-99). Briefly, the mice received 5-day a week treatment, and continued for 4-6 months. At the end treatment, the mice were euthanized, and their prostates were collected for biopsies to examine development of PIN and low/high grade prostate carcinoma.

FIG. 9 illustrates results demonstrating the effect of vitamin E isomers on prostate cells. (A) Cell viability was examined by MTT assay after treatment with different vitamin-E isomers for 24 and 48 hrs. Note that vitamin-E isomers, particularly tocotrienols, affect selectively the viability of the prostate cancer cells at different degree, but do not have significant effect on the non-tumorigenic prostate epithelial cells. PC-3 is more responsive to vitamin-E isomers compared to LNCaP. (B) LNCaP and PC-3 growth rate in the presence of γ-T3 at IC₅₀. The IC₅₀ dose levels correspond to that in FIG. 9A. For alpha-T3, 100 μM was used. UD indicates undetermined IC₅₀.

FIG. 10 illustrates results demonstrating the induction of apoptosis by γ-T3 treatment. (A) Cell cycle analysis by flow cytometry. Control cells and treated cells incubated with γ-T3 at IC₅₀ for 24-hr were subjected to flow cytometry analysis. Note that the sub-G1 population appears after treatment. (B) IC₅₀ time-dependent and 24-hr dose-dependent activation (in hrs and μM respectively) of the pro-apoptosis pathway in PC-3. Note that γ-T3 induces activation of the critical molecules (cleaved caspase 3, 7, 8, 9, PARD) and modulate the ratio between the amounts of bcl-2 and bax in a cell dose- and time-dependent fashion. (C) IC₅₀ γ-T3 activates pro-apoptotic genes and suppresses pro-survival genes expression on LNCaP and PC-3 but not on non-tumorigenic prostate epithelial cells (PZ-HPV) for 24-hr incubation period.

FIG. 11 illustrates results demonstrating the inactivation of pro-survival pathways by γ-T3. (A) Effect of γ-T3 on the activity of NF-κB pathway was examined by IC₅₀ time-dependent and 24-hr dose-dependent western blotting (in hrs and μM respectively). Note that nuclear translocation of NF-κB p65 and phosphorylated iκB were inhibited by γ-T3 treatment. (B) Treatment of γ-T3 also resulted in downregulation of Id family proteins and EGFR in PC-3 cells.

FIG. 12 illustrates results demonstrating that the Jun N-terminal Kinase (JNK) activation is involved in γ-T3-induced apoptosis. (A) Cell viability, after incubation with γ-T3 and JNK inhibitor (SP600125) for 24-hr, was examined by MTT assay. Note that the addition of JNK inhibitor alleviates the cytotoxicity of γ-T3 in PC-3, suggesting that JNK mediate the anti-proliferation effect of γ-T3. (B) JNK activity after 24-hr dose-dependent and IC₅₀ time-dependent γ-T3 treatment (in μM and hrs respectively) and was found to be elevated by measuring the phosphorylation levels of MKK4, SAPK/JNK, c-jun and ATF-2. Thus, confirming the involvement of JNK in γ-T3 anti-cancer property.

FIG. 13 illustrates results demonstrating the inhibition of cell invasion by γ-T3 treatment. (A) 24-hr dose-dependent and IC₅₀ time-dependent γ-T3 treatment induces the expression of epithelial markers (E-Cadherin, γ-catenin), but suppresses the expression of mesenchymal markers (vimentin, twist and α-SMA) and E-cadherin's repressor (snail). (B) The invasive androgen-independent PCa cells (PC-3) treated with the indicated dosage of γ-T3 was harvested and then plated into the Matrigel-coated (0.5 mg/ml) insert. Cells invaded through the membrane were stained with crystal violet and the images were photographed under microscope. After lysed with extraction buffer, intensity at 595 nm was measured.

FIG. 14 illustrates results demonstrating the synergistic effect of γ-T3 on Docetaxel-induced apoptosis. (A) Effect of Docetaxel and γ-T3 co-treatment for 24-hr. Cells were incubated with different dosages of γ-T3 and 100 nM of Docetaxel for 24 hrs. Cell viability was examined by MTT assay. The percentage of apoptotic PC-3 and LNCaP cells following co-treatment of Docetaxel and γ-T3 was significantly higher than that treated with either agent alone. (B) Using western blotting, it was further demonstrated that γ-T3 co-treatment with Docetaxel for 24-hr enhances PC-3 cell apoptosis through activation of pro-apoptotic molecules (cleaved PARP, caspases 3, 7, 8, 9). Additional suppression of proliferation genes were also confirmed for Id-1, EGFR, iκB, NF-κB p65. (C) Proposed T3 anti-cancer pathway in PCa cells, The proposed anti-cancer pathway in melanoma cells is shown separately also in FIG. 26C.

FIG. 15 illustrates results demonstrating induction of apoptosis in breast cancer cells (BCa) by gamma-T3 treatment. (A) IC₅₀ of different vitamin-E isomers was determined by examination of cell viability by MTT assay 24 hrs after the treatment. Note that vitamin-E isomers, particularly beta-, gamma- and delta-T3, selectively inhibit the viability of the BCa cells at different degree, but do not have significant effect on the non-tumorigenic breast epithelial cells. UD represents undetermined IC₅₀ value. (B) Treatment of cells with gamma-T3 (IC₅₀₋₉₀) resulted in an induction of sub-G1 cell population. The proportion of apoptotic cells (sub-G1 fraction) increased in a dose-dependent manner. (C) Gamma-T3 induces DNA fragmentation in MDA-MB-231 cells. Briefly, the cells were harvested and fragmented DNA was extracted and analyzed by electrophoresis in 2% agarose gel containing ethidium bromide. (D) DNA fragmentation induced by gamma-T3 was also detected by terminal deoxynucleotidyl transferase (TUNEL assay) (“Untreated” black image, i.e. no DNA damage can be detected; 20 and 40 μM gamma-T3, apoptotic cells with severe DNA damage appear in green fluorescence by presence of nicks in the DNA which can then be identified by terminal deoxynucleotidyl transferase). (scale bar 25 μm))

FIG. 16 shows results demonstrating the activation of pro-apoptosis molecules by gamma-T3 treatment. (A) gamma-T3 treatment induces activation of the critical apoptotic molecules (cleaved caspase 3, 7, 8, 9, PARP) and modulates the ratio between the amounts of bcl-2 and bax in a cell dose-dependent fashion. (B) gamma-T3 activates pro-apoptotic genes on MCF7 and MDA-MB-231 cells but not on the non-tumorigenic breast epithelial cells (MCF-10A).

FIG. 17 shows results demonstrating inactivation of pro-survival pathways by gamma-T3. (A) Effect of gamma-T3 on the activity of NF-κB pathway was examined by Western blotting. The phosphorylation of IκB was inhibited by gamma-T3 treatment in total cell lysate. Similarly, the nuclear translocated NF-κB p65 was inhibited in nuclear protein extract. (B) Treatment of gamma-T3 resulted in downregulation of the expression of EGFR and Id family proteins in MDA-MB-231 cells. (C) Treatment of gamma-T3 also resulted in downregulation of the upstream regulators of Id1 in MDA-MB-231 cells (Src, Smad1/5/8 and LOX). The focal adhesion kinase activity (Fak) is strongly correlated with LOX activation. (D) MDA-MB-231 cells treated with gamma-T3 were lysed and the lysate was used for immunoprecipitation assay using the anti-Src antibody. Results indicated that physical interaction between Src and Smad1/5/8 was affected by gamma-T3 treatment.

FIG. 18 shows results demonstrating the Jun N-terminal Kinase (JNK) and MAPK/ERK activation during gamma-T3 induced apoptosis. (A) JNK activity was examined by measuring the phosphorylation levels of SAPK/JNK, c-jun and ATF-2 after 24 hours of gamma-T3 treatment. Note that phosphorylation levels of all the proteins were induced by gamma-T3, suggesting that JNK was activated by gamma-T3 treatment. (B) Cell viability, after incubation with gamma-T3 and JNK inhibitor (SP600125) for 24 hours, was examined by MTT assay. Note that the addition of JNK inhibitor alleviates the cytotoxicity of gamma-T3 on MDA-MB-231 cells, suggesting that JNK mediates the anti-proliferation effect of gamma-T3. (C) MAPK/ERK activity, as examined by measuring the phosphorylation levels of Mek1/2, Erk1/2 and Elk1, was found to be elevated after 24 hours of gamma-T3 treatment. (D) Cell viability, after incubation with gamma-T3 and MAPK/ERK inhibitor (U0126/PD98059) for 24 hours, was examined by MTT assay. Note that the addition of MAPK/ERK inhibitors had no impact on the cytotoxicity of gamma-T3 on MDA-MB-231 cells.

FIG. 19 illustrates the results of experiments demonstrating the inhibition of cell invasion by gamma-T3 treatment. (A) MDA-MB-231 cells treated with the indicated dosage of gamma-T3 was harvested and then plated into the matrigel-coated (0.5 mg/ml) insert. Cells invaded through the membrane were stained with crystal violet and the images were photographed under microscope. After lysed with extraction buffer, intensity at 595 nm was measured and presented with the means and standard deviations (Right panel). (B) 24 hours dose-dependent gamma-T3 treatment had no impact on the expression of epithelial markers (α-, β-, γ-catenin), but suppresses the expression of mesenchymal markers (Twist and α-SMA) and E-cadherin's repressor (Snail, Twist). PC-3 represents the androgen independent prostate cancer cell line expressing wild type E-cadherin.

FIG. 20 illustrates the results of experiments demonstrating the synergistic effect of gamma-T3 on Docetaxel-induced apoptosis. (A) Effect of Docetaxel and gamma-T3 co-treatment for 24 hours. Cells were incubated with 50 nM of Docetaxel together with different dosages of gamma-T3 for 24 hours. Cell viability was examined by MTT assay. The viable MDA-MB-231 cells following co-treatment of Docetaxel and gamma-T3 was significantly lower than that treated with either agent alone. (B-C) Using Western blotting, it was further demonstrated that gamma-T3 co-treatment with Docetaxel for 24 hours promote apoptosis of MDA-MB-231/MCF7 cell through activation of pro-apoptotic molecules (cleaved PARP, caspases 3, 7, 8, 9). Suppression of Id-1 and EGFR expressions were also confirmed by Western blotting analysis. Gamma-TP represents gamma tocopherol. (D) Cell viability, after incubation with gamma-T3 and 3-aminoproprinitrile (APN) for 24 hours, was examined by MTT assay. Note that the addition of APN alleviates the cytotoxicity of gamma-T3 on MDA-MB-231 cells. (E) Id1 mRNA was determined to be repressed following gamma-T3 treatment. However, Id1 mRNA was determined to be restored partially following gamma-T3 co-treatment with 3-aminoproprinitrile (APN). Amount of GAPDH was measured as loading control. (F) Gamma T3 co-treatment with 3-aminoproprinitrile (APN) reversed the activation of pro-apoptosis genes (caspases 3, 7, 8, 9 and PARP) and partially restored the constitutive activation of Id1.

FIG. 21 illustrates results demonstrating the effect of vitamin E isomers on melanoma cells. (A) Cell viability was examined by MTT assay after treatment with different vitamin-E isomers for 24 hrs. Note that vitamin-E isomers, particularly tocotrienols, affect the viability of melanoma cells at different degree. (B) C32 growth rate in the presence of γ-T3 at IC₅₀. For alpha-T3, 100 μM was used.

FIG. 22 illustrates results demonstrating the induction of apoptosis by γ-T3 treatment. (A) Cell cycle analysis by flow cytometry. Control cells and treated cells incubated with γ-T3 at IC₅₀ for 24-hr were subjected to flow cytometry analysis. Note that the sub-G1 population appears after treatment. (B) Dose-dependent (in μM) activation of the pro-apoptosis pathway in C32 and G361. Note that γ-T3 induces activation of the critical molecules (cleaved caspases 3, 7, 9, PARP) in a cell dose-dependent fashion for 24-hr incubation period.

FIG. 23 illustrates results demonstrating the inactivation of pro-survival pathways by γ-T3 in C32 cells. (A) Effect of γ-T3 (in μM) on the activity of NF-κB pathway was examined by western blotting. Note that nuclear translocation of NF-κB p65 and phosphorylated iκB were inhibited by γ-T3 treatment. (B) Treatment of γ-T3 (in μM) also resulted in downregulation of Id family proteins and EGFR in C32 cells.

FIG. 24 illustrates results demonstrating that Jun N-terminal Kinase (JNK) activation is involved in γ-T3-induced apoptosis in C32 cells. (A) Cell viability, after incubation with γ-T3 and JNK inhibitor (SP600125) for 24-hr, was examined by MTT assay. Note that the addition of JNK inhibitor alleviates the cytotoxicity of γ-T3 in C32, suggesting that JNK mediate the anti-proliferation effect of γ-T3. (B) JNK activity after γ-T3 treatment was found to be elevated by measuring the phosphorylation levels of SAPK/JNK, c-jun and ATF2. Thus, confirming the involvement of JNK in γ-T3 anti-cancer property.

FIG. 25 illustrates results demonstrating the inhibition of cell invasion by γ-T3 treatment in malignant melanoma G361. (A) G361 cells treated with the indicated dosage of γ-T3 was harvested and then plated into the Matrigel-coated (0.5 mg/ml) insert. Cells invaded through the membrane were photographed under microscope. After lysed with extraction buffer, intensity was measured at 595 nm. (B) γ-T3 treatment induces the expression of epithelial markers (E-Cadherin and γ-catenin); but suppresses the expression of mesenchymal markers (vimentin, α-SMA and twist). G361 cells treated with different dosages of γ-T3 for 24 hrs were lysed and analyzed with western blotting.

FIG. 26 illustrates results demonstrating the synergistic effect of γ-T3 on Docetaxel- and Dacarbazine-induced apoptosis in C32. (A) Effect of Docetaxel and γ-T3 co-treatment. C32 cells were incubated with 40 μM of γ-T3 and 50 nM/500 μM of Docetaxel/Dacarbazine respectively for 24 hrs. Cell viability was examined by MTT assay. The percentage of viable C32 cells relative to control following co-treatment of Docetaxel and γ-T3 was significantly lower than that treated with either agent alone. (B) Using western blotting, it was further demonstrated that γ-T3 co-treatment with either Docetaxel or Dacarbazine enhances C32 cell apoptosis through activation of pro-apoptotic molecules (cleaved PARP, caspases 3, 7, 9). Additional suppression of proliferation genes were also confirmed for Id-1, EGFR, phosphor-iκB in C32 cells. (C) Proposed T3 anti-cancer pathway in melanoma cells. The proposed anti-cancer pathway in PCa cells is shown separately also in FIG. 12C.

FIG. 27 illustrates results of experiments demonstrating pharmacokinetics, single acute toxicity and serum biomarkers. (A) Forty 5-week old C57BL/6 black mice received single dose intraperitoneal (i.p.) injection containing 1 mg of gamma-tocotrienol. Five mice were sacrificed at different time points (10 min, 30 min, 1 h, 3 h, 6 h, 24 h, 48 h and 72 h). γ-Tocotrienol concentration in serum was analyzed using HPLC method described in material and method. (B) Ninety C57BL/6 black mice (ten for each group) received single dose i.p. injection containing 1, 2, 4, 8, 12, 16, 20, 30 and 40 mg of gamma-tocotrienol in 100 μl injection volume. The weight and survival of mice were observed for 30-day, followed by euthanized by CO₂ inhalation. (C) ten C57BL/6 black mice received 5 dose i.p. injections per week containing 1 mg of gamma-tocotrienol or DMSO blank. Mice were sacrificed by cardiac bleed and serum subjected to biomarkers detection methods described in materials and methods. There were no toxicological changes in any of the parameters examined. Serum level of the biomarkers are albumin (Alb), creatine (Cre), alanine transaminase (ALT), aspartate aminotransferase (AST), urea (Ure) and alkaline phosphatase (ALP) (RANDOX laboratories Ltd, Crumlin, United Kingdom).

FIG. 28 illustrates results of experiments demonstrating body weight, tumor size and organ distribution of the administered γ-T3. Male BALB/c athymic nude mice were implanted with PCa cells and selected randomly into three groups (n=5 per group); control (DMSO as vehicle), gamma-T3 (50 mg/kg/d) and combination treatment of gamma-T3 and Docetaxel (50 mg of gamma-T3/kg/d, and 7.5 mg of Docetaxel/kg/wk). The mice were weighed (A) and the tumors were measured (B) using a Digital Carbon Fiber Caliper (Fisher scientific, Pittsburgh, Pa.) before each drug treatment. (C) gamma-T3 concentration in organs and serum were analyzed using HPLC method described in material and method.

FIG. 29 illustrates imaging of PCa cells xenografted on male BALB/c athymic nude mice following drugs treatment. (A-B) For two repeated experiments, male BALB/c athymic nude mice were implanted with PCa cells and selected randomly into three groups (n=10 per group); control (DMSO as vehicle), gamma-T3 (50 mg/kg/d) and combination treatment of gamma-T3 and Docetaxel (50 mg of gamma-T3/kg/d, and 7.5 mg of Docetaxel/kg/wk). Mice received i.p. injection with luciferin solution (150 mg/kg of body weight). (A1) shows a side view of s.c. PC3-Luc tumor bearing nude mice were treated with DMSO (solvent), single agent (1.5 mg of γ-T3/d/mice) or combination therapy (1.5 mg of γ-T3/d/mice and 0.75 mg of docetaxel/wk/mice). 2 million of PC3-Luc cells were inoculated in male nude mice and the tumor suppression was monitored using IVIS™ Imaging System (Xenogen Corp., Hopkinton, Mass., USA) 5 min after administration of luciferin. (B1) shows a side view of s.c. PC3-Luc tumor bearing nude mice which were treated with DMSO (solvent), single agent (1.0 mg of γ-T3/d/mice) or combination therapy (1.0 mg of γ-T3/d/mice and 0.15 mg of docetaxel/wk/mice). 1 million of PC3-Luc cells were inoculated in male nude mice and the tumor suppression was monitored using IVIS™ Imaging System at the end of the treatment. (A2) & (B2) Average in vivo signal intensity of mice in different treatment groups. (A3) & (B3) Photographs of representative tumors in control, γ-T3, and combination treatment of γ-T3 and docetaxel. Arrow indicates in situ tumors on the nude mice. (A4) & (B4) Photographs of representative the sizes of removed tumors from the control, γT3 as well as γT3 and docetaxel groups.

FIG. 30 shows images illustrating the γ-T3 antitumor effect on cancer cell proliferation. The downregulation of PCNA, Ki67 and Id-1 were determined by IHC immunohistochemistry with mouse antibodies against PCNA, Ki67 and Id-1 and secondary antibody anti-mouse Fab-HRP. The expression level for these three cell proliferation molecules were lower after treatment with either gamma-T3 alone or co-treatment with Docetaxel (Doce). (scale bar in all images 100 μm ______)

FIG. 31 shows images illustrating the gamma-T3 antitumor effect on cancer cell apoptosis. The presence of cleaved caspase 3 and cleaved PARP were determined by IHC immunohistochemistry with rabbit polyclonal antibodies against cleaved caspase-3 and cleaved PARP and secondary antibody anti-rabbit Fab-HRP. The expression level for these two molecules was higher after treatment with either gamma-T3 alone or co-treatment with Docetaxel (Doce). (scale bar in all images 100 μm ______)

FIG. 32 shows images illustrating the gamma-T3 antitumor effect on tumor suppressor gene and its repressor. The changes in expression of tumor suppressor gene (E-cadherin; (A)) and its repressor (Snail; (B)) were determined by IHC immunohistochemistry with antibodies against E-cadherin and Snail and secondary antibody Fab-HRP. The expression level for these two molecules correlates oppositely after treatment with either gamma-T3 or co-treatment with Docetaxel (Doce). (scale bar in all images 100 μm ______)

### DETAILED DESCRIPTION OF THE PRESENT INVENTION

In a first aspect the present invention refers to a method of preventing cancer or preventing the recurrence of cancer after undergoing a cancer treatment by administering a composition comprising at least one of γ-tocotrienol or δ-tocotrienol.

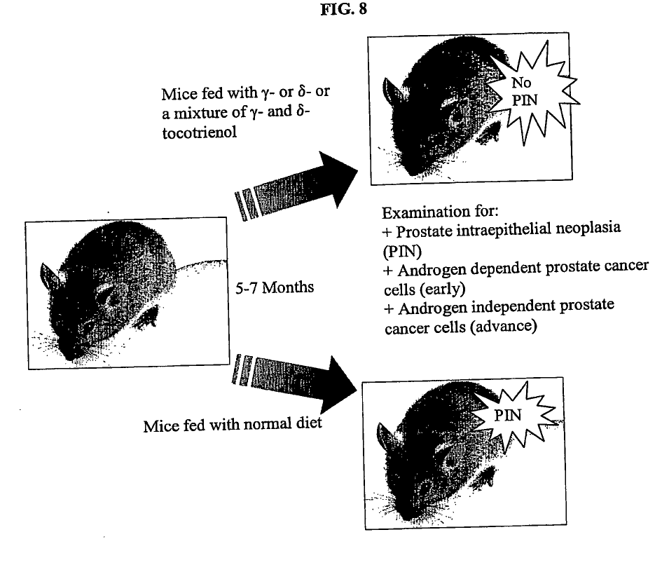

The inventors demonstrate for the first time that a composition referred to herein can a) down-regulate the expression of stem cell markers, such as stem cell markers CD133 and CD44 as evident from Western blotting and flow cytometry analysis referred to herein, and b) suppresses sphere- and tumor formation. Thus, it was demonstrated by the inventors that pre-treatment of cells which might develop into cancer cells with a composition referred to herein was found to interfere with the tumor initiation ability of the cells. These findings are supported by in vitro as well as in vivo data as becomes apparent from the experimental results referred to herein. The general principal of this aspect of the present invention is illustrated in FIG. 7 based on an example in which a composition comprising at least one of gamma- and delta-tocotrienol has been used to prevent the treatment of cancer or to avoid the recurrence of cancer after undergoing a cancer treatment. In a further example, illustrated in FIG. 8 special mice which are genetically modified to develop prostate intraepithelial neoplasia (PIN) do not develop PIN if they are fed with a composition comprising at least one of gamma- or delta-tocotrienol or a mixture of gamma- and delta-tocotrienol.

With “prevention of cancer” it is referred to the act of preventing or hindering cancer from occurring. In the present case, administering a composition referred to herein has the effect that cancer cannot develop in an animal body. Prevention is to be differentiated from “cancer treatment” in which a composition referred to herein would be used for the treatment of cancer cells which already exist in the animal body or in other words for the treatment of an animal body already suffering from cancer. Sometimes the term “chemoprevention” is used. Like the term cancer treatment, “chemoprevention” also refers to the treatment of a patient already suffering from cancer and is not to be mistaken with the “prevention of cancer” as referred to in the claims of the present invention. Chemoprevention indicates that a treatment is supposed to avoid the use of chemotherapy which has mostly severe side effects for the animal body undergoing this specific kind of treatment.

In another embodiment, the composition referred to herein can also be used for preventing recurrence of cancer after undergoing a cancer treatment. That means that an animal body which suffered from cancer and which underwent a treatment to heal the animal body from cancer uses the composition referred to herein to prevent cancer from reoccurring. In one embodiment it means that the animal body underwent and finished a treatment to heal or cure the animal body from cancer. The difference to an ongoing cancer treatment is based on the fact that the composition referred to herein is not used to destroy or stop proliferation of cancer cells but, as for the “prevention of cancer” to prevent or hinder the cancer from reoccurring. “Cure” or “heal” as referred to herein is defined clinically as the permanent absence of signs or symptoms of cancer; complete remission or complete response as disappearance of clinical evidence of cancer.

“Cancer treatment” refers to any kind of known treatment of cancer which aims at eliminating or removing cancer cells. The major modalities of cancer treatment or therapy are surgery, and radiation therapy (for local and local-regional disease), and chemotherapy (for systemic disease). Other important methods include hormonal therapy (for selected cancers, such as prostate cancer, breast cancer or endometrium), immunotherapy (monoclonal antibodies, interferons, and other biologic response modifiers and tumor vaccines), the use of differentiating agents, such as retinoids, agents that exploit the growing knowledge of cellular and molecular biology and mixtures of the aforementioned treatments or therapies.

In general, “cancer” is considered to refer to a group of cells (usually derived from a single cell) that has lost its normal control mechanisms and thus has unregulated growth (proliferation), lack of differentiation, local tissue invasion, and, often, metastasis. Cancerous (malignant) cells can develop from any tissue within any organ. As cancerous cells grow and multiply, they form a mass of cancerous tissue—called the tumor—that can invade and destroy normal adjacent tissues. The term “tumor” refers to an abnormal growth or mass, tumors can be cancerous or noncancerous. Cancerous cells from the primary (initial) site can spread (metastasize) throughout the body. Cancerous cells develop from healthy cells in a complex process called transformation. The first step in the process is initiation, in which a change in the cells genetic material (in the DNA or sometimes in the chromosome structure) primes the cell to become cancerous. The change in the cell's genetic material may occur spontaneously or be brought on by an agent that causes cancer (a carcinogen). The compositions referred to herein which comprise at least one of γ-tocotrienol or δ-tocotrienol can prevent this initiation.

In one embodiment, the types of cancer which can be treated using the composition referred to herein can be cancer caused by genetic mutation(s), such as chromosomal abnormalities, or cancer caused by viruses, such as papilloma viruses, Epstein-Barr virus to name only a few. Two major groups of genes responsible for genetic mutations are oncogenes and tumor suppressor genes. Oncogenes are abnormal forms of normal genes (proto-oncogenes) that regulate cell growth while tumor suppressor genes are inherent genes that play a role in cell division and DNA repair and are critical for detecting inappropriate growth signals in cells. Thus, in one embodiment, cancer to be treated refers either to cancer caused by the mutation of oncogenes or to cancer caused by the mutation of tumor suppressor genes.

In another embodiment, the type of cancer can include, but is not limited to lymphocytic leukemia, myeloid leukemia, malignant lymphoma, myeloproliferative diseases, or solid tumors. In still another embodiment, cancer refers to a type of cancer which can include, but is not limited to melanoma (skin cancer), prostate cancer, colon cancer, liver cancer, bladder cancer, breast cancer and lung cancer. In one example, cancer refers to prostate cancer, breast cancer or melanoma (skin cancer). In a further embodiment, the present invention is directed to the prevention of prostate intraepithelial neoplasia (PIN) or the recurrence of prostate intraepithelial neoplasia (PIN) after undergoing a cancer treatment by administering a composition comprising at least one of γ-tocotrienol or δ-tocotrienol.

Vitamin E is composed of two main components—Tocopherols (T) and Tocotrienols (T3). Tocotrienols (T3) are found mainly in palm oil. Together with tocopherols (T), they provide a significant source of anti-oxidant activity to all living cells. This common anti-oxidant attribute reflects the similarity in chemical structures of the tocotrienols and the tocopherols, which differ only in their structural side chain (contains farnesyl for tocotrienol or saturated phytyl side chain for tocopherol). The common hydrogen atom from the hydroxyl group on the chromanol ring acts to scavenge the chain-propagating peroxyl free radicals. Depending on the locations of methyl groups on their chromanol ring, tocopherols and tocotrienols can be distinguished into four isomeric forms: alpha (α), beta (β), gamma (γ), and delta (δ).

As described, for the prevention of cancer or for the prevention of reoccurrence of cancer after undergoing a cancer treatment a composition comprising at least one of γ-tocotrienol or δ-tocotrienol is used. γ-Tocotrienol and δ-tocotrienol are isoforms of Vitamin E. Vitamin E is composed of two main components—Tocopherols (T) and Tocotrienols (T3). Tocotrienols (T3) are found mainly in palm oil. Together with tocopherols (T), they provide a significant source of anti-oxidant activity to all living cells. This common anti-oxidant attribute reflects the similarity in chemical structures of the tocotrienols and the tocopherols, which differ only in their structural side chain (contains farnesyl for tocotrienol or saturated phytyl side chain for tocopherol).

Different tocopherol and tocotrienol isoforms exist (see Formula I and II). Tocopherols consist of a chromanol ring and a 15-carbon tail derived from homogentisate (HGA) and phytyl diphosphate, respectively. On the other hand, tocotrienols differ structurally from tocopherols by the presence of three trans double bonds in the hydrocarbon tail. Formula I and Formula II and the description following it provide an overview about the known isoforms of tocopherols (T) and tocotrienols (T3).

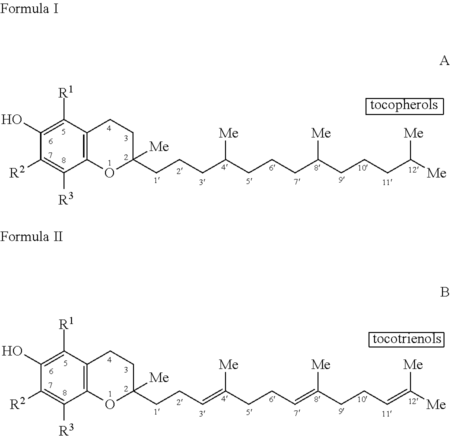

Formula I (A): R1=R2=R3=Me, known as α(alpha)-tocopherol, is designated α-tocopherol or 5,7,8-trimethyltocol; R1=R3=Me; R2=H, known as, β(beta)-tocopherol, is designated, β-tocopherol or 5,8-dimethyltocol; R1=H; R2=R3=Me, known as γ(gamma)-tocopherol, is designated γ-tocopherol or 7,8-dimethyltocol; R1=R2=H; R3=Me, known as δ(delta)-tocopherol, is designated δ-tocopherol or 8-methyltocol. Formula II (B): R1=R2=R3=H, 2-methyl-2-(4,8,12-trimethyltrideca-3,7,11-trienyl)chroman-6-ol, is designated tocotrienol; R1=R2=R3=Me, formerly known as ζ1 or ζ2-tocopherol, is designated 5,7,8-trimethyltocotrienol or α(alpha)-tocotrienol. The name tocochromanol-3 has also been used; R1=R3=Me; R2=H, formerly known as ε-tocopherol, is designated 5,8-dimethyltocotrienol or β(beta)-tocotrienol; R1=H; R2=R3=Me, formerly known as γ-tocopherol, is designated 7,8-dimethyltocotrienol or (gamma)γ-tocotrienol. The name plastochromanol-3 has also been used; R1=R2=H; R3=Me is designated 8-methyltocotrienol or δ(delta)-tocotrienol.

The composition referred to herein comprises or consists of either gamma-tocotrienol or delta-tocotrienol or both isoforms, i.e. a mixture of gamma-tocotrienol and delta-tocotrienol. In one embodiment, the amount of gamma- or delta-tocotrienol can be enriched. “Enriched” means that the respective isoform(s) of tocotrienol is comprised in an amount which is higher than in the normal mixture comprising all isoforms of tocotrienol isolated from its natural source. For example, tocotrienol isolated from, e.g., palm oil, comprises γ-tocotrienol and σ-tocotrienol in an amount of less than 10 wt. % based on the total weight of the oil. Thus, with respect to the embodiments of the present invention, an “enriched” formulation means any formulation comprising γ-tocotrienol or σ-tocotrienol or a mixture of γ-tocotrienol and σ-tocotrienol in an amount of more than 10 wt. % based on the total weight of the formulation (or composition). For example, an enriched formulation comprises γ-tocotrienol or σ-tocotrienol in an amount of at least 10 wt. %. In another example, it comprises a mixture of γ-tocotrienol and σ-tocotrienol, wherein γ-tocotrienol is comprised in an amount of 4 wt. % and σ-tocotrienol in an amount of 6 wt. %, i.e. together 10 wt. %.

In another embodiment enriched means that even in a mixture of γ-tocotrienol and σ-tocotrienol both components are comprised in an amount of at least 10 wt. %, i.e. at least 10 wt. % γ-tocotrienol and at least 10 wt. % σ-tocotrienol (total of 20 wt. % of the total composition).

In another embodiment, enriched tocotrienol composition or formulation refers to a Composition or formulation comprising gamma or delta tocotrienol in an amount of at least 10 wt. % or at least 20 wt. % or at least 30 wt. % or at least 40 wt. % or at least 50 wt. % or at least 60 wt. % or at least 70 wt. % or at least 80 wt. % or at least 90 wt. % based on the total weight of the composition.

In still another embodiment, enriched gamma- and/or delta-tocotrienol composition or formulation refers to a composition or formulation comprising at least one of this tocotrienol isoforms in an amount of about 10 wt. %, 20 wt. %, 30 wt. %, 40 wt. %, 50 wt. %, 60 wt. %, 70 wt. %, 80 wt. % or at least 90 wt. % based on the total weight of the formulation. In a further embodiment, the composition referred to herein can comprise gamma- and delta-tocotrienol together in an amount as specified above.

In another embodiment, the composition can include γ-tocotrienol and δ-tocotrienol in a ratio of 1:Y wherein Y is less than 10. For example, γ-tocotrienol and δ-tocotrienol isolated from, e.g., palm oil, comprises γ-tocotrienol and δ-tocotrienol in a ratio of 1:0.38; annatto oil, comprises γ-tocotrienol and δ-tocotrienol in a ratio of 1:9. Thus, in a further embodiment the composition can include γ-tocotrienol and δ-tocotrienol in a ratio of 1:(0.3 to about 0.7) or 1:(4 to 9). Since many natural products can also comprise other isoforms of tocotrienol the composition referred to herein cannot only comprise only γ-tocotrienol and δ-tocotrienol but can further comprise α-tocotrienol or β-tocotrienol or α-tocotrienol and β-tocotrienol. In another example, the composition referred to herein is substantially free of α-tocotrienol and/or β-tocotrienol and/or α-tocotrienol and β-tocotrienol and/or any tocopherol. However, in one embodiment, it is also possible that at least one tocopherol, such as α-, β-, γ- or δ-tocopherol is comprised in the composition referred to herein. For example, palm oil which has been isolated and enriched to comprise tocotrienols and tocopherols in an amount of 70 wt. % of the total weight of the palm oil can comprise α-tocopherol, α-tocotrienol, β-tocotrienol, γ-tocotrienol and δ-tocotrienol in a ratio of 0.24:0.24:0.033:0.33:0.13.

In one embodiment, the composition comprising at least one γ-tocotrienol and δ-tocotrienol for preventing cancer or preventing the recurrence of cancer does not include a further anti-cancer active agent. In the context of this embodiment, anti-cancer active agent refers to any substance which itself acts to prevent cancer, such as doxorubixin, paclitaxel, tumor necrosis factors (TNF).

In another embodiment, the compositions of the present invention can comprise further substances, green tea polyphenols, such as epicatechin (EC), epigallocatechin (EGC), epicatechin gallate (ECG), and epigallocatechin gallate (EGCG), or organosulfur compounds, such as S-allylmercaptocysteine derived from garlic and allicin derived from garlic, or protein-bound polysaccharides, such as polysaccharide-K (Krestin, PSK) and polysaccharide peptide (PSP) isolated from Trametes versicolor and Coriolus versicolor respectively, or red carotenoid pigments, such as lycopene found in tomatoes and other red fruits & vegetables.

The amount of composition administered to the animal body can be between about 10 mg and about 1000 mg per 60-kg adult or between about 10 mg and about 500 mg per 60-kg adult.

In another embodiment, the composition is administered in an amount to obtain a serum level concentration of an individual tocotrienol isomer in the blood of an animal between about 0.1 to 30 mg/L or between about 10 to 30 mg/L. In one example the concentration of gamma-tocotrienol is about 1 mg/L.

In one embodiment, the animal body is a mammal. Examples for mammals include, but are not limited to a human, pig, horse, mouse, rat, cow, dog or cat.

In another aspect, the present invention refers to a composition comprising at least one of γ-tocotrienol or δ-tocotrienol and (2R,3S)—N-carboxy-3-phenylisoserine; N-tert-butylester, 13-ester with 5,20-epoxy-1,2,4,7,10,13-hexahydroxytax-11-en-9-one-4-acetate-2-benzoate, trihydrate (Docetaxel) and/or (5Z)-5-(dimethylaminohydrazinylidene)imidazole-4-carboxamide (Dacarbazine).

For example, prostate cancer (PCa) is responsible for the largest number of death cases among all other cancers, except for lung cancer. Due to the slow growing nature of the tumor, many of the prostate cancer patients have already developed metastatic disease upon diagnosis and will inevitably enter the hormone refractory stage after hormone ablation therapy. There remains no curative treatment against Hormone refractory prostate cancer (HRPC) at present. The most effective treatment regime for HRPC patients, the Docetaxel-based chemotherapy, can only improve the median survival time for 3 months. Therefore, effective treatment strategies against metastatic HRPC are urgently needed.

To-date, the reason behind the failure of current therapies towards metastatic HRPC is not completely understood, however, increasing evidences had put forward that current therapies are only successful in targeting the more differentiated tumor cells but spare the putative cancer stem/progenitor cells. As for normal stem cells, cancer stem cells (CSCs) are thought to be quiescent comparing to the mature cancer cells. This property makes CSCs resistance to chemotherapeutic drugs which target mainly the actively replicating cells. In addition, prostate CSCs do not express androgen receptor. Thus, they do not respond to hormone ablation as mature tumor cells do. Owning to the self renewing and differentiation ability, they are capable of regenerating the heterogeneous tumor population (with both androgen dependent and independent cells) after hormone ablation which accounts for tumor relapse. Using the composition of the present invention comprising at least one of γ-tocotrienol or δ-tocotrienol together with Docetaxel, it has been shown for the first time that cancer, such as prostate cancer, can be successfully treated as indicated by in vitro and in vivo results referred to herein.

The inventors demonstrated herein for the first time that Docetaxel- or Dacarbazine-induced apoptosis was found to be significant enhanced in the presence of a composition referred to herein, suggesting a synergistic effect between a composition comprising at least one of γ-tocotrienol or δ-tocotrienol and Docetaxel and/or Dacarbazine against cancer cells, such as melanoma cells, breast cancer cells and prostate cancer cells. The lethal dose 50 (LD₅₀) study performed by the inventors indicated no toxicity after treatment with tocotrienols extract (LD₅₀≧2000 mg/kg), the results from this study show that tocotrienol isomers can be used as a safe and effective anti-cancer agent in combination with a chemotherapeutic drug, such as Docetaxel and Dacarbazine, for the treatment of cancer, such as malignant melanoma, breast cancer, liver cancer, bladder cancer, lung cancer, colon cancer or prostate cancer.

The results of the experiments referred to herein confirm for the first time the involvement of JNK pathway in tocotrienol, such as gamma-tocotrienol, induced apoptosis in cancer cells, such as melanoma cells or breast cancer cells or prostate cancer cells. Worth noting is that, the JNK pathway is also known to be involved in cell apoptosis induced by the chemotherapeutic drug, Docetaxel and Dacarbazine. Taking these findings into consideration, it was therefore questioned by the inventors whether tocotrienol possesses synergistic interaction with Docetaxel and Dacarbazine as a result of activation of JNK pathway. To this end, the inventors compared the anti-proliferation capability of a chemotherapeutic drug alone, or co-treatment with tocotrienol. Remarkably, it was found that combined treatment of a chemotherapeutic drug and tocotrienol, but not tocopherol, such as gamma-tocopherol, resulted in higher proportion of apoptotic cells.

The inventors also found that the compositions referred to herein can modulate the activity of at least one protein of the Id family, such as Id-1, Id-2, Id-3 or Id-4. In one embodiment, the compositions referred to herein inhibit the activity of Id-1. It was also found that the compositions referred to herein inhibit cell invasion, i.e. cancer metastasis, through restoration of E-cadherin and gamma-catenin expression. Thus, in one embodiment, the compositions comprising the at least one of γ-tocotrienol or δ-tocotrienol and Docetaxel and/or Dacarbazine inhibits metastazation of cancer.

Docetaxel which can be used in combination with the tocotrienol enriched composition or formulation referred to herein is an anti-neoplastic medication used for example for the treatment of breast, ovarian, and non-small cell lung cancer. Docetaxel is marketed under the name Taxotere® Injection Concentrate by Sanofi-Aventis. Docetaxel is administered as a one-hour infusion every three weeks generally over a ten cycle course. Docetaxel is of the chemotherapy drug class; taxane, and is a semi-synthetic analogue of Taxol (paclitaxel), an extract from the rare Western yew tree Taxus brevifolia. The anti-cancer activity of docetaxel is due to promoting and stabilising microtubule assembly, while preventing physiological microtubule depolymerisation/disassembly in the absence of GTP. This leads to a significant decrease in free tubulin, needed for microtubule formation and results in inhibition of mitotic cell division between metaphase and anaphase, preventing further cancer cell division and growth.

The other chemotherapeutic agent which can be used in combination with the tocotrienol enriched composition or formulation referred to herein is Dacarbazine (DTIC). Dacarbazine belongs to the group of alkylating agents. Dacarbazine is a triazene derivative with antineoplastic activity. Dacarbazine alkylates and cross-links DNA during all phases of the cell cycle, resulting in disruption of DNA function, cell cycle arrest, and apoptosis. As such, Dacarbazine is used for the treatment of various cancers, among them malignant melanoma, Hodgkin lymphoma, sarcoma, and islet cell carcinoma of the pancreas, to name only a few.

In another embodiment, it is referred to a method of inhibiting or arresting or reversing of cancer by administering a composition comprising at least one of γ-tocotrienol or δ-tocotrienol together with (2R,3S)—N-carboxy-N-tert-butylester-3-phenylisoserine, 13-ester with 5,20-epoxy-1,2,4,7,10,13-hexahydroxytax-11-en-9-one-4-acetate-2-benzoate, trihydrate (Docetaxel) and/or (5Z)-5-(dimethylaminohydrazinylidene)imidazole-4-carboxamide (Dacarbazine).

In the context of the present invention, “reversing” means to reduce the size of the tumor mass or masses and finally to eliminate the tumor completely. Thus, reversing cancer means to cure the cancer, i.e. to eliminate any signs of cancer in the animal body. “Inhibiting” or “arresting” cancer means to stabilize the tumor. A stabilized tumor indicates neither improvement nor worsening of the disease.

The part of the composition comprising at least one of γ-tocotrienol or δ-tocotrienol can be used in the same formulations, amounts, combination with other substances (polyphenols etc.) as described above with respect to the first aspect. The composition or formulation comprising at least one of γ-tocotrienol or δ-tocotrienol can be either administered separately to the chemotherapeutic drug, i.e. Docetaxel and/or Dacarbazine or they can be formulated together in one composition.

In the method of inhibiting or arresting or reversing of cancer, the cancer can be in the form of melanoma (skin cancer), prostate cancer, colon cancer, prostate intraepithelial neoplasia, bladder cancer, liver cancer, breast cancer or lung cancer.

In one embodiment, it is referred to a method of inhibiting or reversing of melanoma, wherein the composition comprises at least one of γ-tocotrienol or δ-tocotrienol together with (2R,3S)—N-carboxy-N-tert-butylester-3-phenylisoserine, 13-ester with 5,20-epoxy-1,2,4,7,10,13-hexahydroxytax-11-en-9-one-4-acetate-2-benzoate, trihydrate (Docetaxel) and/or (5Z)-5-(dimethylaminohydrazinylidene)imidazole-4-carboxamide (Dacarbazine). In still another embodiment, it is referred to a method of inhibiting or reversing of prostate cancer, or breast cancer or prostate intraepithelial neoplasia, wherein the composition comprises at least one of γ-tocotrienol or δ-tocotrienol together with (2R,3S)—N-carboxy-N-tert-butylester-3-phenylisoserine, 13-ester with 5,20-epoxy-1,2,4,7,10,13-hexahydroxytax-11-en-9-one-4-acetate-2-benzoate, trihydrate (Docetaxel). In still another embodiment, the present invention refers to the use of a composition comprising at least one of γ-tocotrienol or δ-tocotrienol together with (2R,3S)—N-carboxy-3-phenylisoserine, N-tert-butylester, 13-ester with 5,20-epoxy-1,2,4,7,10,13-hexahydroxytax-11-en-9-one-4-acetate-2-benzoate, trihydrate (Docetaxel) or (5Z)-5-(dimethylaminohydrazinylidene)imidazole-4-carboxamide (Dacarbazine) for the manufacture of a medicament for the treatment of cancer or of cancer types as described above.

Since vitamin E and their isoforms are in general water insoluble, the compositions referred to herein are in one embodiment prepared in a water soluble form. Thus, the compositions referred to herein are water solubilized by the addition of specific compounds. A water solubilized form of a composition referred to herein can be obtained, for example, by formulating it into a solid dispersion. Other methods of formulating water-dispersible or water-soluble tocotrienol forms are disclosed for example in U.S. 5,869,704.

The term “solid dispersion” defines a system in a solid state (as opposed to a liquid or gaseous state) comprising at least two components, wherein one component is dispersed throughout the other component or components. For example, the active ingredient (tocotrienols) or combination of active ingredients (tocotrienols and chemotherapeutic drug) is dispersed in a matrix comprised of a pharmaceutically acceptable water-soluble polymer(s) and a pharmaceutically acceptable surfactant(s).

The term “solid dispersion” encompasses systems having small particles of one phase dispersed in another phase. These particles are typically of less than 400 μm in size, for example less than 100 μm, 10 μm, or 1 μm in size. When said dispersion of the components is such that the system is chemically and physically uniform or homogenous throughout or consists of one phase (as defined in thermodynamics), such a solid dispersion will be called a “solid solution” or a “glassy solution.” A glassy solution is a homogeneous, glassy system in which a solute is dissolved in a glassy solvent.

Such solid dispersions can be administered via different routes. For example, orally administered solid dosage forms include but are not limited to capsules, dragées, granules, pills, powders, and tablets. Excipients commonly used to formulate such dosage forms include encapsulating materials or formulation additives such as absorption accelerators, antioxidants, binders, buffers, coating agents, colouring agents, diluents, disintegrating agents, emulsifiers, extenders, fillers, flavouring agents, humectants, lubricants, preservatives, propellants, releasing agents, sterilizing agents, sweeteners, solubilizers, and mixtures thereof.

Excipients for orally administered compounds in solid dosage forms can include, but are not limited to agar, alginic acid, aluminium hydroxide, benzyl benzoate, 1,3-butylene glycol, castor oil, cellulose, cellulose acetate, cocoa butter, corn starch, corn oil, cottonseed oil, ethanol, ethyl acetate, ethyl carbonate, ethyl cellulose, ethyl laureate, ethyl oleate, gelatine, germ oil, glucose, glycerol, groundnut oil, isopropanol, isotonic saline, lactose, magnesium hydroxide, magnesium stearate, malt, olive oil, peanut oil, potassium phosphate salts, potato starch, propylene glycol, talc, tragacanth, water, safflower oil, sesame oil, sodium carboxymethyl cellulose, sodium lauryl sulfate, sodium phosphate salts, soybean oil, sucrose, tetrahydro fur fury 1 alcohol, and mixtures thereof.

In one embodiment, a dosage form can comprise a solid solution or solid dispersion of at least one γ-tocotrienol and/or δ-tocotrienol or a mixture of at least one γ-tocotrienol and/or δ-tocotrienol together with Docetaxel and/or Dacarbazine in a matrix, and the matrix can comprise at least one pharmaceutically acceptable water-soluble polymer and at least one pharmaceutically acceptable surfactant. Suitable pharmaceutically acceptable water-soluble polymers include, but are not limited to, water-soluble polymers having a glass transition temperature (T_{g}) of at least 50° C., or at least 60° C., or from about 80° C. to about 180° C.

Water-soluble polymers having a T_{g} as defined above allow for the preparation of solid solutions or solid dispersions that are mechanically stable and, within ordinary temperature ranges, sufficiently temperature stable so that the solid solutions or solid dispersions can be used as dosage forms without further processing or be compacted to tablets with only a small amount of tableting aids.

The water-soluble polymer comprised in a dosage form referred to herein is a polymer that can have an apparent viscosity, when dissolved at 20° C. in an aqueous solution at 2% (w/v), of 1 to 5000 mPa s, or of 1 to 700 mPa s, or of 5 mPa s to 100 mPa s.

Water-soluble polymers suitable for use in a dosage form referred to herein can include, but are not limited to homopolymers and copolymers of N-vinyl lactams, especially homopolymers and copolymers of N-vinyl pyrrolidone, e.g. polyvinylpyrrolidone (PVP), copolymers of N-vinyl pyrrolidone and vinyl acetate or vinyl propionate; cellulose esters and cellulose ethers, in particular methylcellulose and ethylcellulose, hydroxyalkylcelluloses, in particular hydroxypropylcellulose, hydroxyalkylalkylcelluloses, in particular hydroxypropylmethylcellulose, cellulose phthalates or succinates, in particular cellulose acetate phthalate and hydroxypropylmethylcellulose phthalate, hydroxypropylmethylcellulose succinate or hydroxypropylmethylcellulose acetate succinate; high molecular polyalkylene oxides such as polyethylene oxide and polypropylene oxide and copolymers of ethylene oxide and propylene oxide; polyacrylates and polymethacrylates such as methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), poly(hydroxyalkyl methacrylates); polyacrylamides, vinyl acetate polymers such as copolymers of vinyl acetate and crotonic acid, partially hydrolyzed polyvinyl acetate (also referred to as partially saponified “polyvinyl alcohol”), polyvinyl alcohol; oligo- and polysaccharides such as carrageenans, galactomannans and xanthan gum, or mixtures of one or more thereof.

The term “pharmaceutically acceptable surfactant” as used herein refers to a pharmaceutically acceptable non-ionic surfactant. A dosage form referred to herein comprises at least one surfactant having a hydrophilic lipophilic balance (HLB) value of from 12 to 18, or from 13 to 17, or from 14 to 16. The HLB system attributes numeric values to surfactants, with lipophilic substances receiving lower HLB values and hydrophilic substances receiving higher HLB values.

In one embodiment, a dosage form referred to herein comprises one or more pharmaceutically acceptable surfactants selected from polyoxy ethylene castor oil derivates, e.g. polyoxyethyleneglycerol triricinoleate or polyoxyl 35 castor oil (Cremophor® EL) or polyoxyethyleneglycerol oxystearate such as polyethylenglycol 40 hydrogenated castor oil (Cremophor® RH 40, also known as polyoxyl 40 hydrogenated castor oil or macrogolglycerol hydroxystearate) or polyethylenglycol 60 hydrogenated castor oil (Cremophor® RH 60); or a mono fatty acid ester of polyoxy ethylene (20) sorbitan, e.g. polyoxyethylene (20) sorbitan monooleate (Tween® 80), polyoxyethylene (20) sorbitan monostearate (Tween® 60), polyoxyethylene (20) sorbitan monopalmitate (Tween® 40), or polyoxyethylene (20) sorbitan monolaurate (Tween® 20). Other surfactants including those with HLB values of greater than 18 or less than 12 may also be used, e.g., block copolymers of ethylene oxide and propylene oxide, also known as polyoxyethylene polyoxypropylene block copolymers or polyoxyethylene polypropyleneglycol, such as Poloxamer® 124, Poloxamer® 188, Poloxamer® 237, Poloxamer® 388, or Poloxamer® 407.

Where two or more surfactants are used, the surfactant(s) having an HLB value of from 12 to 18 preferably accounts for at least 50% by weight, more preferably at least 60% by weight, of the total amount of surfactants used.

A dosage form referred to herein can also include additional excipients or additives such as flow regulators, lubricants, bulking agents (fillers) and disintegrants. Such additional excipients may comprise, without limitation, from 0% to 15% by weight of the total dosage form.

Dosage forms referred to herein can be provided as dosage forms consisting of several layers, for example laminated or multilayer tablets. They can be in open or closed form. “Closed dosage forms” are those in which one layer is completely surrounded by at least one other layer. Multilayer forms have the advantage that two active ingredients which are incompatible with one another can be processed, or that the release characteristics of the active ingredient(s) can be controlled. For example, it is possible to provide an initial dose by including an active ingredient in one of the outer layers, and a maintenance dose by including the active ingredient in the inner layer(s). Multilayer tablets, types may be produced by compressing two or more layers of granules.

Furthermore, a film coat on the tablet can contribute to the ease with which a tablet can be swallowed. A film coat also improves taste and provides an elegant appearance. If desired, the film-coat may be an enteric coat. The film-coat usually includes a polymeric film-forming material such as hydroxypropyl methylcellulose, hydroxypropylcellulose, and acrylate or methacrylate copolymers. Besides a film-forming polymer, the film-coat may further comprise a plasticizer, e.g. polyethylene glycol, a surfactant, e.g. a Tween® type, and optionally a pigment, e.g. titanium dioxide or iron oxides. The film-coating may also comprise talc as anti-adhesive. The film coat usually accounts for less than 5% by weight of the dosage form.

Other specific forms of formulating the compositions referred to herein, include, but are not limited to native oil liquids of tocotrienols, such as palm oil, which can be used for the manufacture of a soft gel, a water soluble emulsion liquid form, which can be used for the manufacture of soft drinks, a cold water dispersible powder, which can be used for the manufacture of soft capsules and tablets, or beadlets, which can be used for the manufacture of hard capsules.

For the manufacture of the compositions referred to herein in form of water soluble emulsion liquid, tocotrienol liquids are used as starting material to which one adds glycerine and blends of emulsifiers. Afterwards the mixture is homogenized into an emulsion.

Examples for emulsifiers which can be used for the formulation of water soluble emulsion liquid include, but are not limited to glycerine fatty acid esters, acetic acid esters of monoglycerides, lactic acid esters of monoglycerides, citric acid esters of monoglycerides, succinic acid esters of monoglycerides, diacetyl tartaric acid esters of monoglycerides, polyglycerol esters of fatty acids, polyglycerol polyricinoleate, sorbitan esters of fatty acids, propylene glycol esters of fatty acids, starch derivatives, surfactants, sucrose esters of fatty acids, calcium stearoyl di lactate, lecithin, or enzyme digested lecithin/enzyme treated lecithin.

Cold water dispersible powders of the compositions referred to herein can be manufactured by providing tocotrienol oil liquids as starting material. Emulsifiers, such as modified corn starch, maltodextrin, cyclodextrins or corn starch, are added to the tocotrienol oil. The mixture can afterwards be spray dried into a dry powder.

Beadlets comprising compositions referred to herein can be obtained by providing tocotrienol oil liquids as starting material. Afterwards, gelatine, corn starch, sucrose and ascorbyl palmitate are added in one embodiment to the tocotrienol oil. The mixture is spray dried into dry beadlets.

Injectable formulations which allow the introduction and delivery of the above compositions into the circulatory system of the animal body via subcutaneous, intramuscular or intraperitoneal (i.p.) injections in precisely calculated dosages. Propylene glycol is a commonly used solvent for such formulations. In another embodiment the compositions are formulated in a water-in-oil formulation.

Thus, in one embodiment the compositions referred to herein are administered in the form of as a tablet, beadlet, or (soft) gel, or dragée, or sustained-release formulation, or ointment, or injectable formulation or in encapsulated form. Encapsulated forms for example can include compositions encapsulated in phospholipids.

In still another aspect, the present invention refers to a method of manufacturing any of the compositions or formulations referred to herein. Any known method of formulating such compositions can be used. Thus, in one embodiment the method of manufacturing such a compositions comprises mixing at least one γ-tocotrienol or δ-tocotrienol with (2R,3S)—N-carboxy-3-phenylisoserine, N-tert-butylester, 13-ester with 5,20-epoxy-1,2,4,7,10,13-hexahydroxytax-11-en-9-one-4-acetate-2-benzoate, trihydrate (Docetaxel) and/or (5Z)-5-(dimethylaminohydrazinylidene)imidazole-4-carboxamide (Dacarbazine).

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Experimental Section

1. In the following experiments, the anti-proliferative effect of the different tocopherol and tocotrienol isomers using melanoma cell lines was compared. It was found that all the eight vitamin-E isomers can inhibit the proliferation of malignant skin cancer cells. The potency varied between each isomer, with the γ-T3 being the most potent. Detailed study of the effect of γ-T3 revealed that γ-T3 treatment of the two melanoma cell lines resulted in induction of apoptosis, which was associated with the downregulation of the pro-survival factors such as Id-1 or EGFR. Meanwhile, activation of JNK and inactivation of NF-κB were also observed after the treatment. Inhibition of JNK activity by specific inhibitor, SP600125, blocked partially the sensitivity to γ-T3 treatment, indicating that the γ-T3-induced apoptosis is mediated through JNK signalling pathway. In addition, γ-T3 treatment also restored the expression of E-cadherin, γ-catenin and suppressed the expression of mesenchymal markers in melanoma cells, resulting in inhibition of cell invasion. Interestingly, Docetaxel- or Dacarbazine-induced apoptosis was found to be significant enhanced in the presence of γ-T3, demonstrating a synergistic effect between tocotrienol isoforms, such as γ-T3 and chemotherapy drugs (Docetaxel and Dacarbazine) against melanoma cells. Since previous reports and our lethal dose 50 (LD₅₀) study (data not shown) indicated no toxicity after treatment with tocotrienols extract (LD₅₀≧2000 mg/kg), the results from this study suggest that T3 isomers can be used as a safe and effective anti-cancer agent either alone or in combination with other chemotherapeutic drug for the treatment of malignant melanoma.

1.1 Materials and Experimental Conditions

1.1.1 Melanoma cell lines, cell culture conditions and chemicals—Amelanotic melanoma (C32), malignant melanoma (G361) cells (ATCC, Rockville, Md.) were maintained in their respective medium recommended by ATCC (Invitrogen, Carlsbad, Calif.) supplemented with 2 mmol/1 L-glutamine, 10% fetal calf serum (FCS) and 1% penicillin streptomycin at 37° C. in 5% CO₂. Docetaxel, Dacarbazine (Calbiochem) and JNK inhibitor (Sigma-Aldrich) were dissolved in dimethylsulfoxide (DMSO). The treatment solutions were diluted in culture medium to obtain the desired concentrations.

1.1.2 Tocotrienol and tocopherol isomers were extracted and purified from palm oil using Davos Life Science (Singapore) separation technology. Crude palm oil (CPO) feed was purchased from Kuala Lumpur Kepong Berhad. Using the corresponding tocotrienol isomers as the reference standard, the purity of T3 and T isomers was verified to be 97% by high performance liquid chromatography (HPLC) percentage area (%-area).

1.1.3 Cell viability study and time course experiment—For cell viability study, 1×10⁴ cells resuspended in 100 μl medium were plated into each well of a 96-well plate. The cells were then treated with different concentrations of the vitamin-E isomers for 24 hrs. After the treatment, 20 μl of 3-(4,5-Dimethyl thiazol-2-yl)-2,5-diphenyl tetrazolium bromide (MTT) solution was added into each well and the cells were incubated at 37° C. for 2 hrs. The formazan crystals were then re-suspended in 200 μl of DMSO and the intensity at 595 nm was measured. For JNK inhibitor study, cells were pre-treated with 20 μM of SP600125 for 8 hrs prior to the addition of vitamin-E isomers. For time course study, 5×10³ cells were treated with different concentrations of the vitamin-E isomers and were subjected to MTT cell proliferation assay at the indicated time point. If IC₅₀ for the isomer is >100 μM, 100 μM will be used as treatment dosage. Each experiment was repeated three times in triplicate wells and the growth curves showed the means and standard deviations.

To test the effect of γ-T3 on the cytotoxicity of Docetaxel and Dacarbazine, cells were co-incubated with γ-T3 and Docetaxel or Dacarbazine. After 24 hrs, cells were subjected to western blotting and MTT cell proliferation assays.

1.1.3 Flow cytometry—Cell cycle distribution was examined using flow cytometry. Briefly, cells were harvested by trypsinization, fixed in 70% ethanol at 4° C. overnight, and then resuspended in PBS. After incubation at 4° C. for overnight, 2×10⁶ cells were incubated with in 20 μg/ml propidium iodide (PI) and 2 mg RNaseA for 15 minutes at 37° C. Cells were then examined by BD SLRII cytometer and the results were analyzed using ModFit software (Becton Dickinson, Mountain View, Calif.). Data were expressed as the percentage of cell cycle distribution in the entire population.

1.1.4 Matrigel-invasion assay—Matrigel-invasion assay was performed according to a previously published method with modifications. Briefly, cells were pre-incubated in a serum-free RPMI 1640 medium with or without γ-T3 isomers for 24 hrs. Cells (2.5×10⁵) resuspended in 500 μl of serum-free RPMI 1640 containing 0.1% bovine serum albumin (BSA) were then added to the upper chamber of a 8 μm pore size insert (Millipore, Bedford, Mass.) manually coated with Matrigel (0.5 mg/ml) (BD Bioscience, Bedford, Mass.). Five hundred μl of invasion buffer containing fibronectin (10 μg/ml) and RPMI 1640 supplemented with 10% FCS were added in the lower chamber as a chemo-attractant. Cells were incubated at 37° C. for 24 hrs in 5% CO₂ humidified conditions. At the end of incubation period, inserts were stained with Diff-Quick staining solution (Fischer Scientific). Non-invaded cells on the inside of the insert were scraped off with a cotton swab. Cell invasions were then examined by a phase-contrast microscope. The invaded cells were extracted using extraction buffer (Millipore, Bedford, Mass.) and the cell number was estimated based on absorbance at 595 nm.

1.1.5 Western blotting—Detailed protocols have been described previously and are known in the art. Briefly, cell lysates were prepared by suspending cell pellets in lysis buffer [50 mmol/l Tris-HCl (pH 8.0), 150 mmol/l NaCl, 1% NP40, 0.5% deoxycholate, 0.1% SDS, 1 mg/ml aprotinin, 1 μg/ml leupeptin and 1 mmol/l phenylmethylsulfonyl fluoride]. For nuclear protein extraction, NucBuster™ protein extraction kit was used. Protein concentration was measured using the DC Protein Assay kit (Bio-Rad, Hercules, Calif.). Equal amount of protein (30 μg) was loaded onto a 10% SDS polyacrylamide gel for electrophoresis and then transferred onto a polyvinylidene difluoride membrane (Amersham, Piscataway, N.J.). The membrane was then incubated with primary antibodies for 1 hr at room temperature against E-cadherin (BD Biosciences, Bedford, Mass.), α-catenin, β-catenin, γ-catenin, Id-1, Id-3, EGFR, phosphor-c-jun, phosphor-ATF2, cleaved PARP, vimentin, α-smooth muscle actin, twist (Santa Cruz Biotechnology, Calif.), phosphor-IkB-alpha (Ser32/36), phosphor-IKK alpha (Seri 80)/IKK beta (Ser181), phosphor-SAPK/JNK (Thr183/Tyr185) G9, SAPK/JNK, NF-kB p65 (5A5) (Cell Signaling Technology Inc, Beverly, Mass.), Snail (Abcam). After incubation with appropriate secondary antibodies, signals were visualized by ECL western blotting system (Amersham, Piscataway, N.J.). Expression of β-actin and histone H1 were assessed as an internal loading control for total cell lysate and nuclear protein lysate respectively.

1.2 Results—Anti-Proliferation Effect of Vitamin-E Isomers

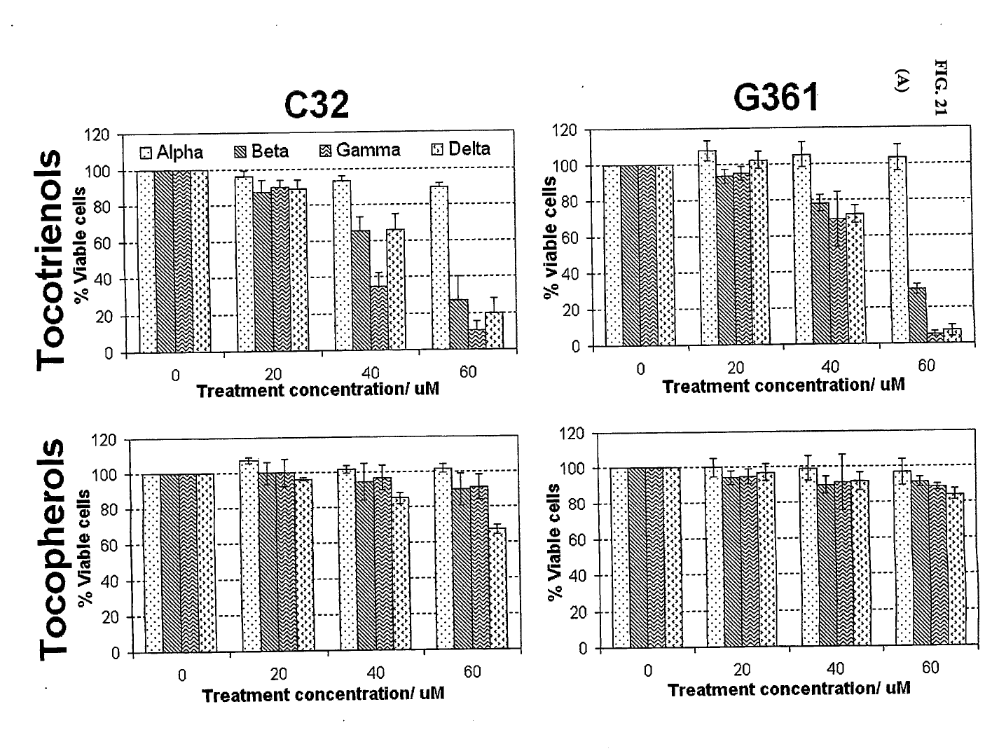

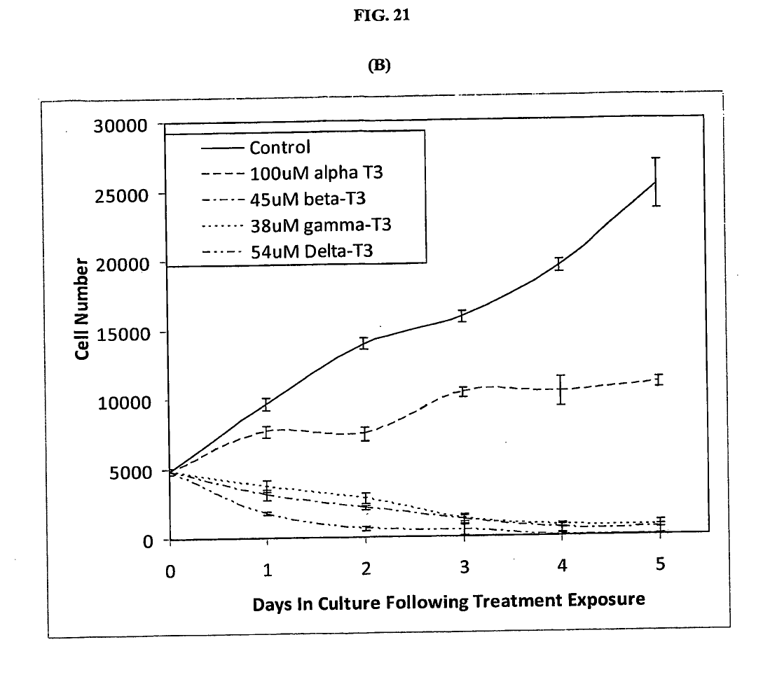

Melanoma cells were treated with vitamin-E isomers at increasing dosage (0, 20, 40 and 60 μM) and for varying time points. The results showed that vitamin-E isomers significantly suppressed the proliferation of skin cancer cells (G361 and C32) (FIG. 21A). The inhibition of cell proliferation was significantly stronger for T3 isomers, particularly for γ-T3, which showed a dose-dependent inhibition (FIG. 21B). Based on the concentration that caused 50% growth inhibition (IC₅₀) in G361 cells, the order of inhibitory effect is γ-T3>δ-T3>β-T3>α-T3≈α-T≈β-T≈γ-T≈δ-T.

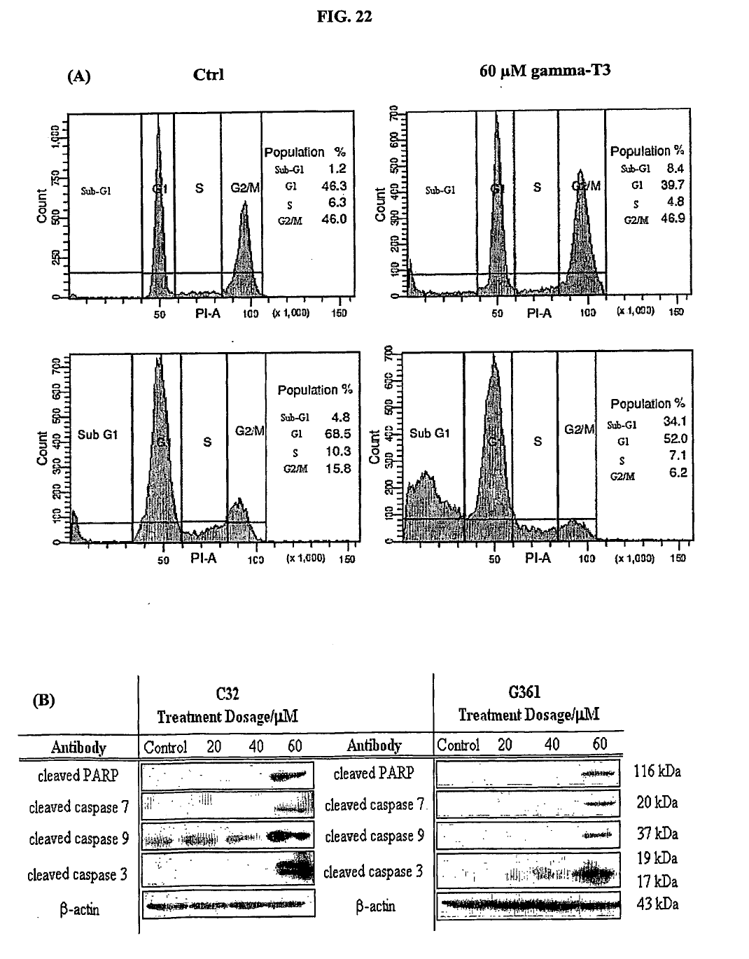

To study the mechanism responsible for γ-T3-induced growth inhibition, cell cycle distribution of the cells with or without γ-T3 treatment were analyzed by flow cytometry. Consequently, treatment of cells with γ-T3 resulted in an induction of sub-G1 cell population, indicating the presence of apoptotic cells after the treatment (FIG. 22A). Consistent with the result of flow cytometry, activation of procaspase 3, 7, 9 as well as PARP, as evidenced by the appearance of the cleaved products, were observed in C32 cells treated with different γ-T3 dosage (FIG. 22B). Meanwhile, these γ-T3-mediated activation of the proapoptotic proteins were in a dose-dependent manner, consistent with the effect of γ-T3 treatment on inhibition of cell proliferation.

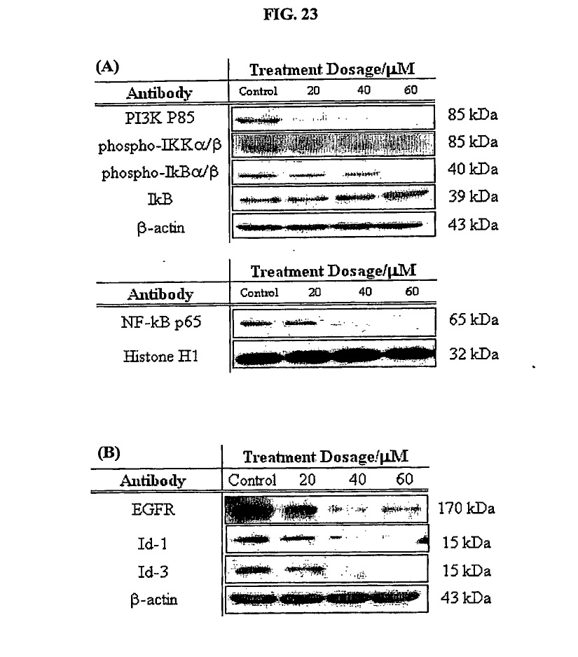

γ-T3 downregulates the pro-survival signalling pathways—Because NF-κB was reported to be constitutively activated in C32, the possibility that γ-T3 induced cell apoptosis attributable to the suppression of NF-κB activation was considered. The NF-κB activities of C32 treated with γ-T3 at different dosages were measured by examining the translocation of NF-κB subunit p65. As illustrated in FIG. 23A, γ-T3 treatment suppressed constitutive NF-κB p65 activity in a dose-dependent manner. The effect of γ-T3 on NF-κB signalling was further explored by examining the expression of other upstream regulators, such as phosphor-iκBα/β, iκBα/β, phospho-IKKα/β and phosphatidylinositol 3-kinase (PI3K). Translocation of NF-κB to nucleus is inhibited by the IκBα/β protein, which are degraded through phosphorylation by IKKα/β. The IKKα/β is in turn phosphorylated and activated by the PI3K. In γ-T3 treated C32 cells, a dose-dependent decrease in the level of the phosphorylated iκBα/β were observed (FIG. 23A). This is associated with the decrease in the level of phosphorylated IKKα/β, IκBα/β, as well as an inhibition of PI3K p85 and NF-κB p65 nuclear translocation. These results indicate that γ-T3 suppressed NF-κB activity through the dephosphorylation and accumulation of IκBα/β.

It was found that γ-T3 treatment also downregulates a number of the key proteins that are involved in the development and progression of skin cancer. As shown in FIG. 23B, Id-1 and Id-3 expression levels were significantly suppressed to almost undetectable level by treatment with increasing dosages of γ-T3. Similar effect on EGF-R protein level was also observed. Since EGF-R and Id protein family are essential for cancer cell growth and survival, their downregulation may be associated with the γ-T3-induced growth arrest and apoptosis.

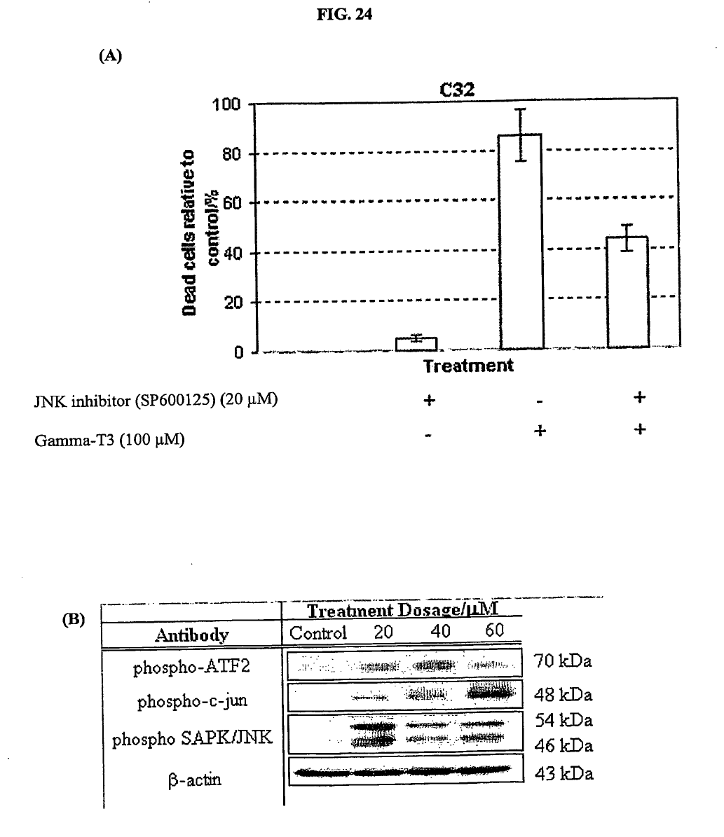

Activation of pro-apoptotic pathway by γ-T3 treatment—The c-Jun N-terminal kinase (JNK) is an evolutionarily conserved serine/threonine protein kinase that is activated by stress and genotoxic agents. JNK phosphorylates the amino terminal of all three Jun transcription factors and ATF-2 members of the AP-1 family. The activated transcription factors modulate gene expression to generate appropriate biological responses, including cell migration and cell death. When C32 cells were treated with varies dosages of γ-T3, a dosage-dependent increase in JNK phosphorylation activities were detected (FIG. 24B). Meanwhile, phosphorylation of the JNK downstream effectors such as ATF-2 or c-jun were all upregulated by γ-T3, supporting that JNK signalling pathway was activated by the γ-T3.

To further confirm the importance of JNK activation in γ-T3 induced apoptosis in melanoma cells, it was investigated whether inactivation of JNK with a specific inhibitor, SP600125, could protect cells from γ-T3. As shown in FIG. 24A, co-treatment of γ-T3 together with 20 μM of SP600125 decreased the percentage of apoptotic cells compared to that treated with γ-T3 alone, confirming that JNK activation may be required for γ-T3-induced apoptosis in C32.

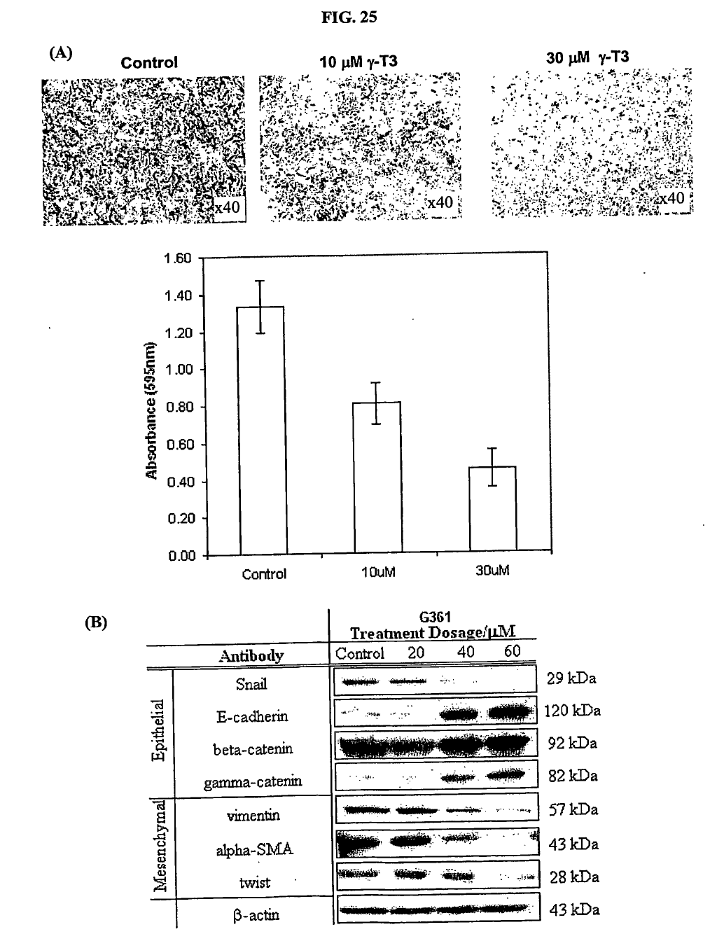

Effect of γ-T3 on inhibition of cell invasion—Although γ-T3 has been shown to have anti-proliferation effect on many cancers, it is not clear if it affects cancer metastasis. Therefore, it was examined whether γ-T3 could suppress the invasive ability of the skin cancer cells. As shown in FIG. 25B, using matrigel-invasion assay, it was found that γ-T3 treated (IC₅₀ and IC₈₅) G361 cells showed a 2-time lower invasion capability compared to the untreated control as evidenced by decreased in the number of cells invaded through the matrigel layer. This inhibitory effect on cell invasion was not the result of cell growth inhibition induced by γ-T3 as the number of viable cells added into the invasion chamber were the same. These results indicate that γ-T3 is able to inhibit the invasion ability of melanoma cells, independent to their cytotoxic effects.

Down-regulation of E-cadherin expression is one of the most frequently reported characteristics of metastatic cancers. Restoration of E-cadherin expression in cancer cells leads to suppression of metastatic ability. In melanoma, down-regulation of E-cadherin expression is correlated with high-grade tumours and poor prognosis, indicating their roles in melanoma progression. Interestingly, it was found that E-cadherin and γ-catenin protein expression were up-regulated whereas E-cadherin's repressor (Snail) were down-regulated after treatment with γ-T3 (FIG. 25A), although the expression for β-catenin remained constant at all treatment dosages. Separately, the mesenchymal markers (vimentin, α-SMA and twist) were all down-regulated after treatment with γ-T3. These results suggested that the inhibitory effect of γ-T3 on cancer cell invasion may act through induction of the E-cadherin, γ-catenin protein expression; and suppression of Snail, vimentin, α-SMA and twist.

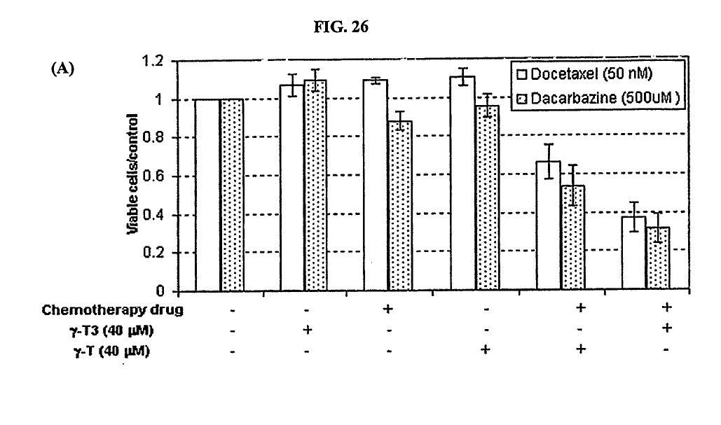

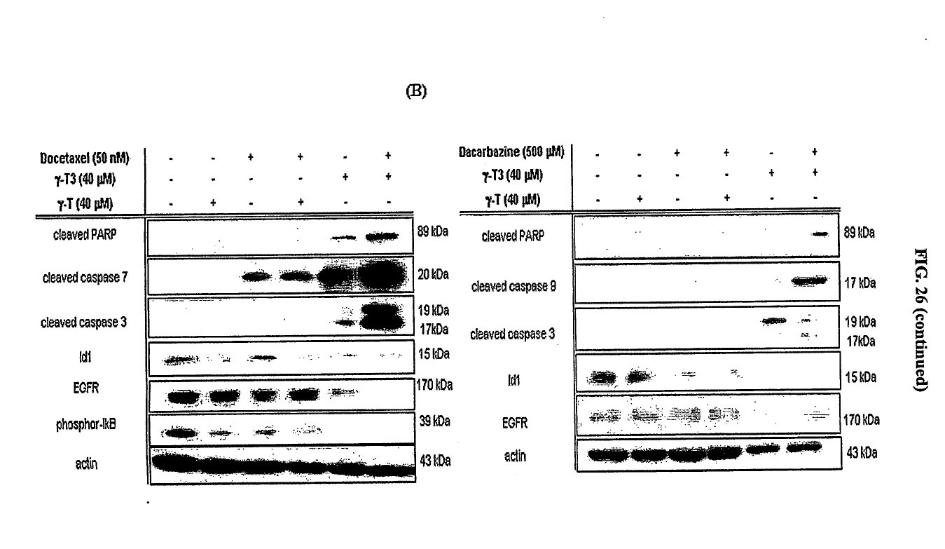

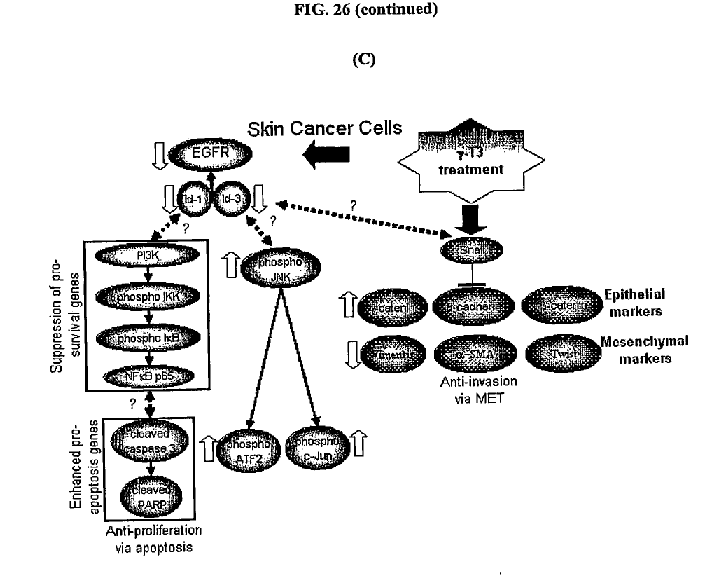

Effect of γ-T3 treatment on Docetaxel- and Dacarbazine-induced apoptosis—To test if γ-T3 can act synergistically with chemotherapeutic agents, the effect of γ-T3 alone or in combination with Docetaxel or Dacarbazine was examined. The latter two represent an important class of anti-cancer agents that are known to have in vitro and in vivo effects against cancers of lung, ovaries, breast, leukemia and malignant melanoma. As shown in FIG. 26A, the percentage of viable cells in C32 cell lines following co-treatment of Docetaxel or Dacarbazine with γ-T3 was significantly lower than that treated with γ-T3 or Docetaxel alone. Using western blotting, it was further demonstrated that γ-T3 co-treatment with either Docetaxel or Dacarbazine enhances cell apoptosis through activation of pro-apoptotic proteins (cleaved PARD, caspases 3, 7) and down-regulation of pro-survival proteins (Id-1, EGFR and phosphor-iκB) (FIG. 26B). The level of apoptotic cells is in stark contrast to that co-treated with γ-T (γ-Tocopherol) and Docetaxel. These results suggested that γ-T3, but not γ-T can act in synergy with Docetaxel or Dacarbazine against skin cancer cells.

1.3 The above experiments show that, among eight vitamin-E isomers, γ-T3 inhibits melanoma cell proliferation through modulation of both pro-survival (Id-1, Id-3, EGFR and NF-κB) and pro-apoptotic (caspases) pathways. Meanwhile, it was demonstrated for the first time that γ-T3 inhibit malignant melanoma cell invasion by restoration of the E-cadherin and γ-catenin expression. This inhibitory effect was also associated with suppression of expression for mesenchymal markers like twist, α-SMA and vimentin. Together with the finding that γ-T3 enhanced the anti-cancer effect of Docetaxel and Dacarbazine, the experiments provide strong evidences that γ-T3 can be used as a safe and effective anti-cancer agent for the treatment of skin cancer.

Furthermore, it was demonstrated that γ-T3 suppressed constitutive NF-κB activity through inhibition of IκB kinase activation, leading to apoptosis in melanoma cells. Consequently, this resulted in induction of apoptosis via activation of caspases 3, 7, 9 and PARP. It is worth noting that γ-T3 was previously demonstrated to abolish NF-κB activation induced by epidermal growth factor (EGF) and other pro-inflammatory cytokines. Although the molecular mechanism involved was not clear at that time, it was proposed that γ-T3 may act through a common step in the suppression of NF-κB. The experimental results referred to herein revealed that down-regulation of EGF receptor (EGF-R) was correlated with γ-T3 induced NF-κB inactivation in skin cancer cells (FIG. 23B).

It was believed that one possible mechanism by which γ-T3 could mediate its effects on the NF-κB pathway is through the suppression of Id-1. Previously, skin melanocytes that constitutively expressing Id-1 were shown to delayed cellular senescence that is not associated with a change in cell growth or telomere length. In addition, it was also determined that elevated Id-1 protein levels were present consistently in radial growth phase melanomas suggesting its role in initiation of carcinogenesis in melanoma. Separately in other cell lines, it was previously demonstrated that ectopic Id-1 expression in prostate cancer cells (LNCaP) resulted in increase of NF-κB transactivation activity and nuclear translocation of the p65 and p50 proteins, which was accompanied by up-regulation of their downstream effectors Bcl-xL and ICAM-1. In addition, inactivation of Id-1 by its anti-sense oligonucleotide and retroviral construct in hormone-independent prostate cancer cells resulted in the decrease of nuclear level of p65 and p50 proteins, which was associated with increased sensitivity to TNF-induced apoptosis. Considering these findings, the results strongly suggest that Id gene family may be one of the upstream regulators of NF-κB that is targeted directly by γ-T3, and inhibition of NF-κB signalling pathway may be responsible for γ-T3 induced anti-proliferation in melanoma cells.

In the above experiments, it was also shown that c-Jun N-terminal kinase (JNK) participates in γ-T3 induced apoptosis. When melanoma cells were treated with γ-T3, a series of molecules associated with JNK pathway, such as c-Jun and ATF-2 (FIG. 24A), were activated simultaneously. Meanwhile, it was demonstrated that treatment of JNK inhibitor (SP600125) protects the melanoma cells from γ-T3 induced apoptosis (FIG. 24A). This further confirms the involvement of JNK pathway in γ-T3 induced apoptosis in melanoma cells. The findings referred to herein demonstrate that γ-T3 can function as a common enhancer on chemotherapeutic agents.

It was determined in the experiments referred to herein that restoration of E-cadherin and γ-catenin expression, together with suppression of snail, α-SMA, vimentin and twist, may account for γ-T3's inhibitory effect on melanoma cell invasion. The results referred to herein provide first evidence to suggest that it can also be a potential agent for suppression of malignant melanoma cell invasion. Down-regulation of epithelial markers (E-cadherin and γ-catenin) and up-regulation of mesenchymal markers (α-SMA, vimentin and twist) are some of the most frequently reported phenomenon in metastatic cancers. It is suggested that loss of E-cadherin expression is able to promote this epithelial-mesenchymal transition (EMT), which plays a key role in the progression of cancer cells to metastatic stage. Although the precise mechanism responsible for E-cadherin inactivation in cancer cells is not clear, alterations at transcriptional level due to its repressor Snail seem to be one of the mechanisms responsible for its decreased expression in several cancer types. In the experiments referred to herein, it was found that the γ-T3-treated melanoma cells showed increased E-cadherin expression (FIG. 25A), which was associated with reduced Snail protein expression and invasion ability (FIG. 25B). Catenins (α,γ), a family of cytoplasmic cadherin binding proteins, link E-cadherin to the actin cytoskeleton and are thought to be essential for normal E-cadherin function. The experiments referred to herein demonstrate that γ-T3 only up-regulated the expression of E-cadherin and γ-catenin, but not α-catenin. The expression for β-catenin remains static. Although γ-T3 treated G361 cells do not show elevated α-catenin expression, a key molecule for functional E-cadherin expression, γ-T3 might restore the function of E-cadherin through other molecules such as vinculin, which has been reported to play a role in the establishment of the E-cadherin-based cell adhesion complex. Taken together, the results suggest that γ-T3 can suppress cancer metastasis through induction of mesenchymal-to-epithelial transition (MET).

As summarized in FIG. 26C, the results demonstrated that γ-T3 is a potent inhibitor of melanoma cell proliferation and invasion which acts through multiple molecular pathways. Since no side effect can be observed after long term intake of natural T3 extract (LD₅₀ ≧2000 mg/kg, data not shown), γ-T3 may be used alone or in combination with chemotherapy for treating Id1-associated malignant melanoma.

2. In the following experiments, the anti-proliferative effect of the different tocopherol and tocotrienol isomers using prostate cancer cell lines was compared and the molecular pathway responsible for their activity was examined. It was shown that the inhibitory effect of gamma(γ)-tocotrienol was most potent, which resulted in induction of apoptosis as evidenced by activation of pro-caspases and the presence of sub-G1 cell population. Examination of the pro-survival genes revealed that the gamma-tocotrienol induced cell death was associated with suppression of NF-κB, EGF-R and Id family proteins (Id1 and Id3). Meanwhile, gamma-tocotrienol treatment also resulted in the induction of JNK signalling pathway and inhibition of JNK activity by specific inhibitor (SP600125) was able to partially block the effect of gamma-tocotrienol. It was also found that gamma-tocotrienol treatment led to suppression of mesenchymal markers and the restoration of E-cadherin and gamma-catenin expression, which was associated with suppression of cell invasion capability. Furthermore, synergistic effect was observed when cells were co-treated with gamma-tocotrienol and chemotherapeutic agents, such as Docetaxel. The results suggested that the anti-proliferative effect of gamma-tocotrienol act through multiple signalling pathways, and demonstrated the anti-invasion and chemosensitization effect of gamma-tocotrienol against PCa cells.

2.1 Materials and Experimental Conditions

2.1.1 Prostate cancer cell lines, cell culture conditions and chemicals—The human androgen-dependent PCa cells (LNCaP), human androgen-independent PCa cells (PC-3) (ATCC, Rockville, Md.) were maintained in their respective medium recommended by ATCC (Invitrogen, Carlsbad, Calif.) supplemented with 2 mmol/l L-glutamine, 10% fetal calf serum (FCS) and 2% penicillin streptomycin at 37° C. in 5% CO₂. The immortalized human prostate epithelial cells (PZ-HPV-7) (ATCC, Rockville, Md.) were maintained in keratinocyte serum free medium (K-SFM) supplemented with bovine pituitary extract (BPE, 0.05 mg/ml) and human recombinant epidermal growth factor (EGF, 5 ng/ml EGF). Docetaxel (Calbiochem) and JNK inhibitor, SP600125 (Sigma-Aldrich), were dissolved in dimethylsulfoxide (DMSO). The treatment solutions were diluted in culture medium to obtain the desired concentrations.

2.1.2 For the following experiments the same tocotrienol and tocopherol isomers have been used as described above under 1.1.2.

2.1.3 Cell viability study and time course experiment—For cell viability study, 5×10³ cells resuspended in 100 μl medium were plated into each well of a 96-well plate. The cells were then treated with different concentrations (20, 40, 60, 80, 100 μM) of the vitamin-E isomers for 24 and 48 hrs. After the treatment, 20 μl of MTT solution was added into each well and the cells were incubated at 37° C. for 2 hrs. The formazan crystals were then re-suspended in 200 μl of DMSO and the intensity at 595 nm were measured. For JNK inhibitor study, cells were pre-treated with 20 μM of SP600125 for 8 hrs prior to the addition of vitamin-E isomers. For time course study, 5×10³ cells (LNCaP and PC3) were treated with IC₅₀ concentrations of the vitamin-E isomers and were subjected to MTT assay at the indicated time point. If IC₅₀ for the isomer is >100 μM, 100 μM will be used as treatment dosage. Each experiment was repeated three times in triplicate wells and the growth curves showed the means and standard deviations.

To test the effect of γ-T3 on the cytotoxicity of Docetaxel, cells were pre-incubated with γ-T3 for 3 hrs before addition of 20 and 100 nM of Docetaxel. After 24 hrs, cells were subjected to western blotting and MTT assays respectively.

2.1.4 Flow cytometry was carried out as described above under item 1.1.3. Matrigel-invasion assay was carried out as described above under item 1.1.4. Western blotting was carried out as described above under item 1.1.5.

2.2 Results—Anti-Proliferation Effect of Vitamin-E Isomers

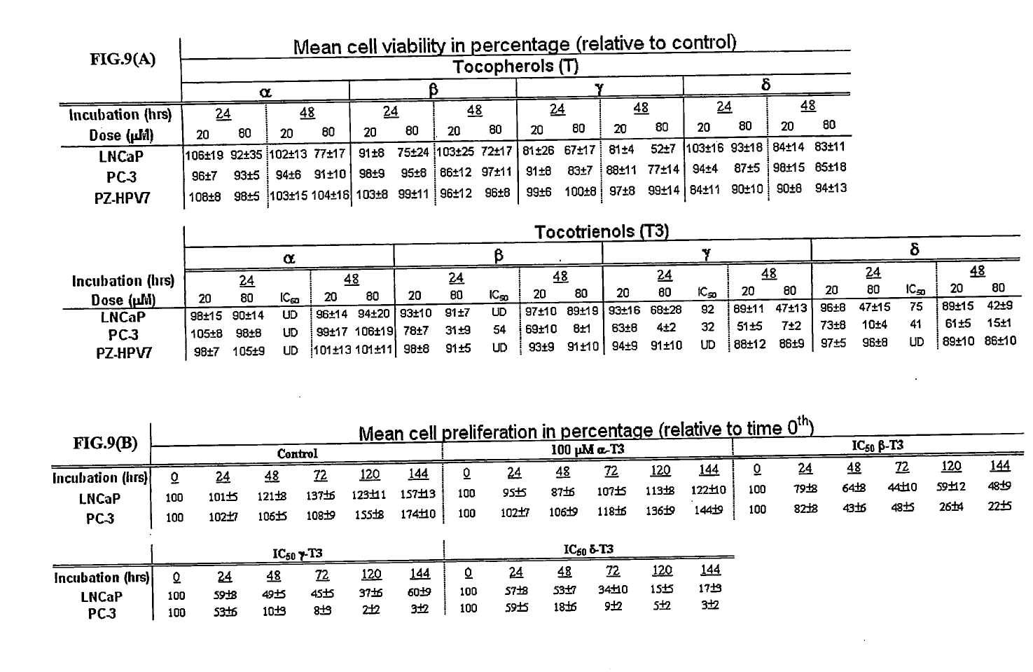

PCa cells were treated with vitamin-E isomers for 24- and 48-hr at increasing dosage (low: 20 μM, medium: 40 μM and high: 80 μM) and for varying time points. The results showed that vitamin-E isomers did not affect significantly the proliferation rate of normal prostate epithelial cells (PZ-HPV-7), but significantly suppressed the proliferation of LNCaP and PC-3 (FIG. 9A). The dose to suppress 50% cell growth (IC₅₀) in LNCaP and PC-3 was inversely proportional to the length of incubation time. Surprisingly, PC-3 cells were more sensitive to the growth inhibition of the vitamin-E isomers than LNCaP cells. The inhibition of cell proliferation was significantly stronger for T3 isomers in PC-3, particularly for γ-T3, which showed a dose and time-dependent inhibition (FIG. 9B). Although δ-T3 was more potent in suppressing cell growth in LNCaP (FIG. 9A), the IC₅₀ value was significantly higher than that for γ-T3 in PC-3. Separately, γ-T was also found to induce apoptosis in LNCaP cells at a dose similar to γ-T3 (data not shown). Based on the IC₅₀ values in PC-3 cells incubated with various isomers for 24-hr, the order of inhibitory effect is γ-T3>δ-T3>β-T3>>γ-T>δ-T≈α-T3≈α-T≈β-T. For the subsequent experiments, the most potent isomer for PC-3 (γ-T3) was investigated since they are in general considered more invasive and resistant to chemotherapeutic agents compared to LNCaP cells.

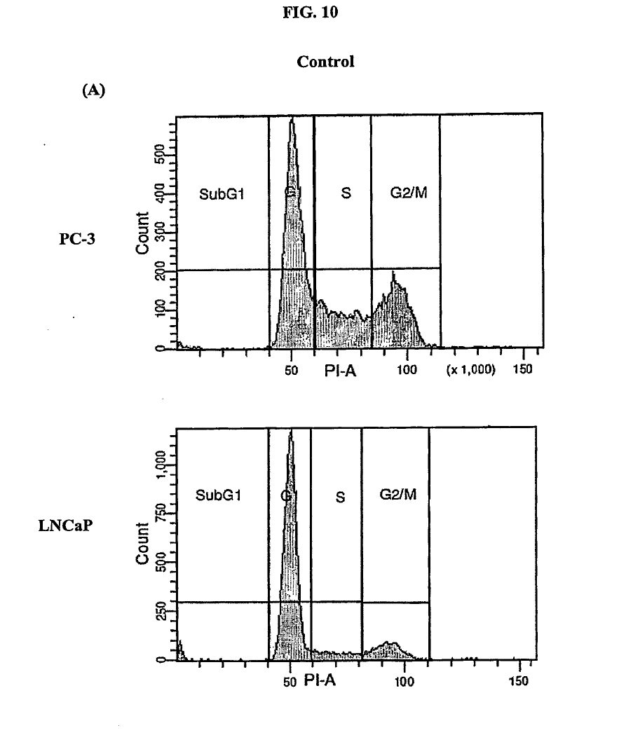

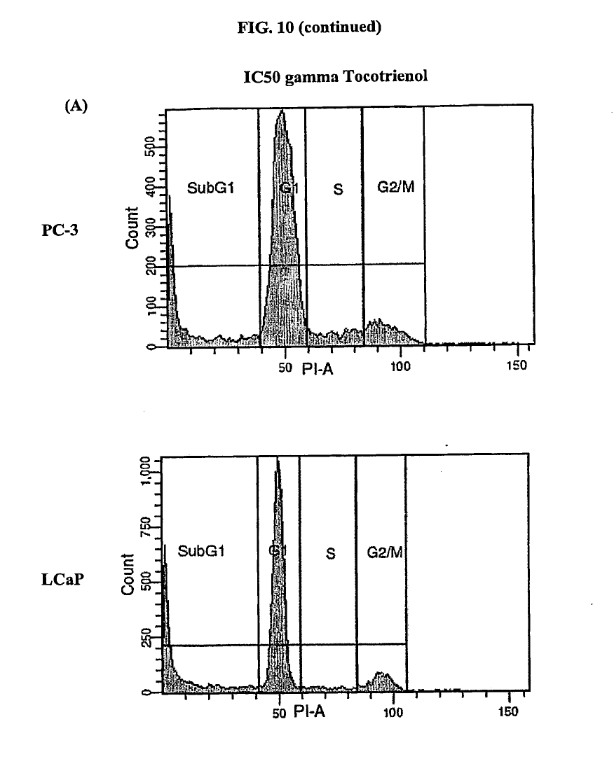

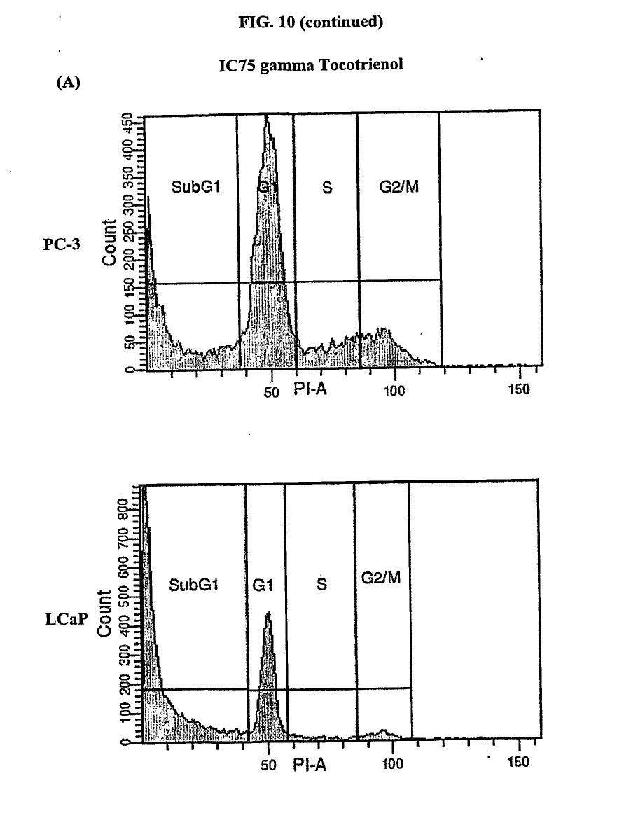

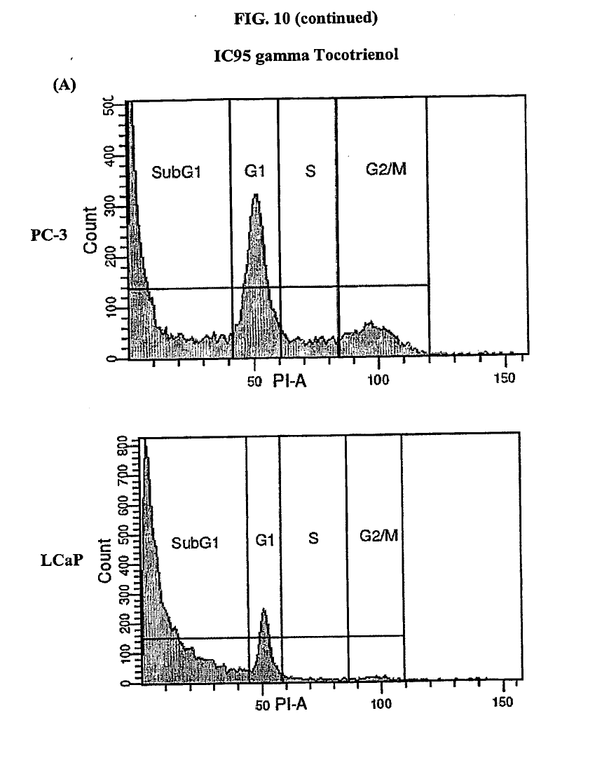

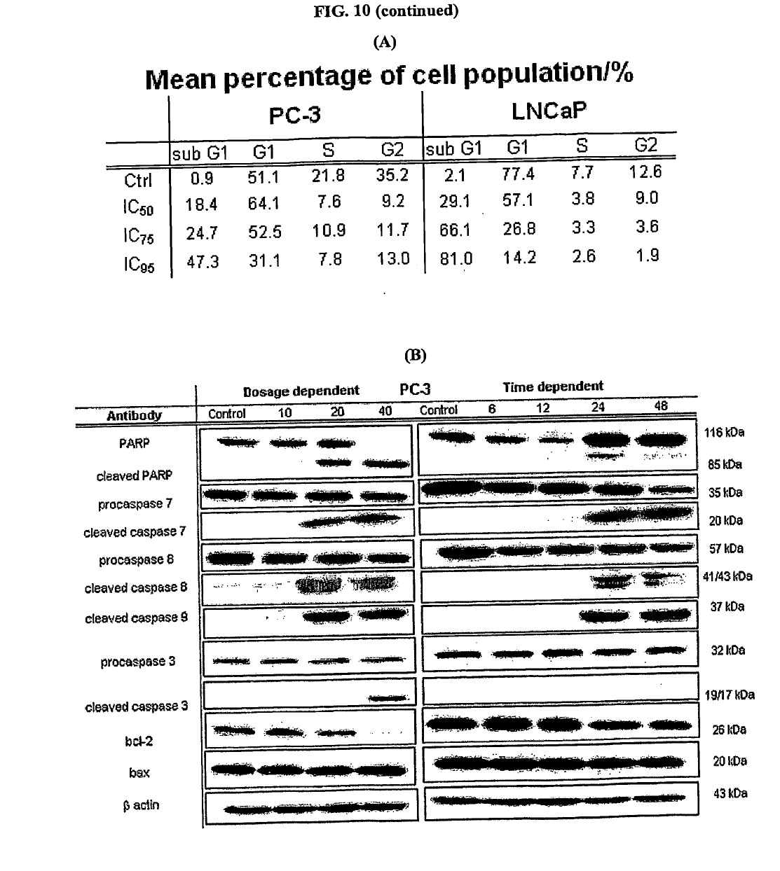

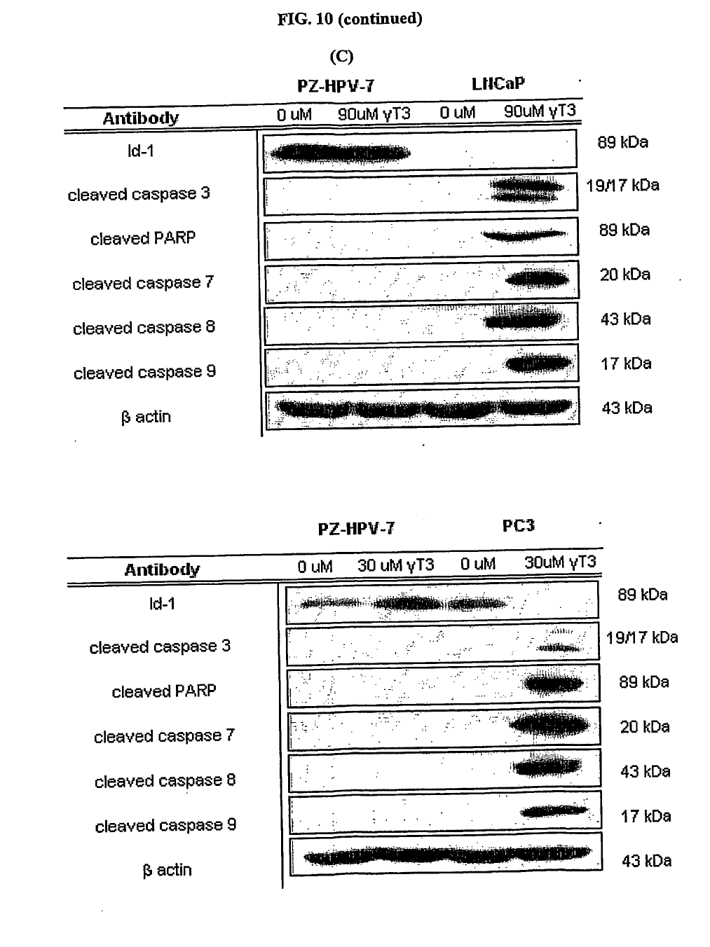

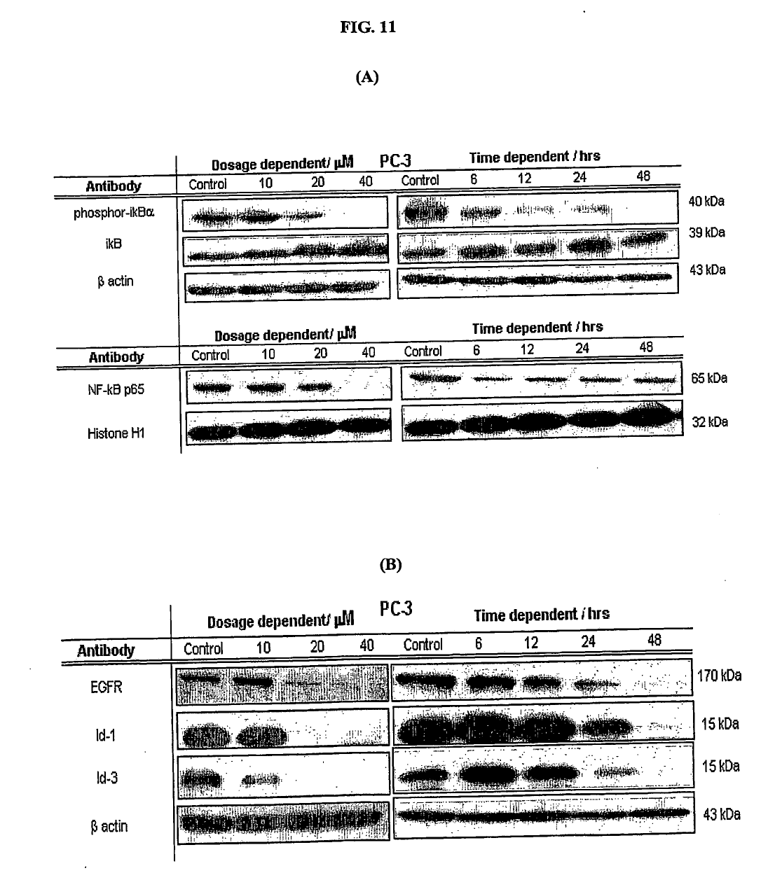

To study the mechanism responsible for γ-T3-induced growth inhibition, cell cycle distribution of the cells with or without γ-T3 treatment for 24 hrs were analyzed by flow cytometry. Consequently, treatment of cells with γ-T3 (IC₅₀₋₉₅) resulted in an induction of sub-G1 cell population, indicating the presence of apoptotic cells after the treatment (FIG. 10A). The proportion of apoptotic cells (sub-G1 fraction) increased in a dose-dependent manner. Worth noting, although γ-T3 was previously reported to induce G1 arrest in some cell lines, a significant increase of G1 population in prostate cancer cells that were treated with γ-T3 was not observed. Consistent with the induction of sub-G1 cell population in flow cytometry, activation of procaspase 3, 7, 8, 9 as well as PARP, as evidenced by the appearance of the cleaved products, were observed in PC-3 cells treated with different γ-T3 dosage for 24 hrs. Downregulation of bcl-2 was also detected after the treatment, although bax expression was not affected, which is likely due to the lack of p53 expression in PC-3 cells (FIG. 10B). Meanwhile, these γ-T3-mediated activation of the proapoptotic proteins as well as the change of bcl-2/Bax ratio were in a dose and time dependent manner (FIG. 10B), consistent with the effect of γ-T3 treatment on inhibition of cell proliferation. In addition, activation of these pro-apoptotic genes after IC_{═}γ-T3 treatment (FIG. 10C) were only observed in PC-3 and LNCaP cells, but not in PZ-HPV-7, indicating that γ-T3 specifically induced apoptosis of androgen-independent prostate cancer cells.

γ-T3 downregulates the pro-survival signalling pathways—Because NF-κB is known to be constitutively activated in PC-3, the possibility that γ-T3 induced cell apoptosis attributable to the suppression of NF-κB activation was considered. The NF-κB activities of PC-3 treated with γ-T3 at either different dosages or at IC₅₀ for different period were measured by examining the translocation of NF-κB subunit p65. As illustrated in FIG. 11A, γ-T3 treatment suppressed constitutive NF-κB p65 activity in a dose-dependent and time-dependent manner. The effect of γ-T3 on NF-κB signalling was further explored by examining the expression of other upstream regulators, such as phosphor-iκBα/β and iκBα/β. In γ-T3 treated PC-3 cells, a time-dependent and dose-dependent decrease in the level of the phosphorylated IκBα/β were observed (FIG. 11A). This is associated with the increase in the level of IκBα/β (named IκBa/b in some Figures), as well as an inhibition of NF-κB p65 nuclear translocation. These results indicate that γ-T3 suppressed NF-κB activity through the dephosphorylation and accumulation of IκBα/β.

It was found that γ-T3 treatment also downregulates a number of the key proteins that are involved in the development and progression of prostate cancer. As shown in FIG. 11B, EGF-R expression was significantly suppressed to almost undetectable level by treatment with increasing dosages of γ-T3. Similar effect on Id-1 and Id-3 protein level was observed. Since EGF-R and Id protein family are essential for cancer cell growth and survival, their downregulation may be associated with the γ-T3-induced growth arrest and apoptosis.

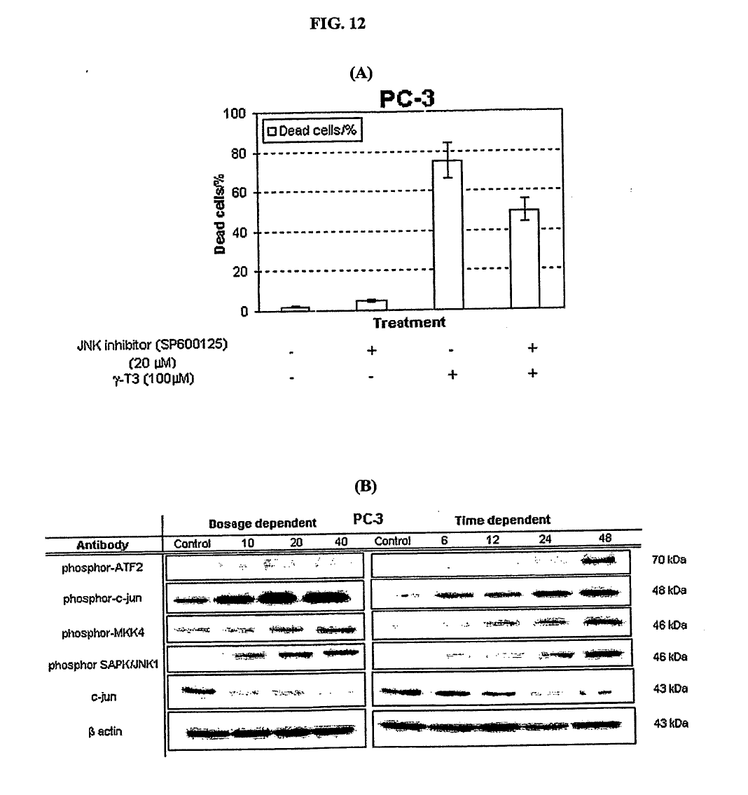

As already mentioned, activation of pro-apoptotic pathway by γ-T3 treatment—The c-Jun N-terminal kinase is an evolutionarily conserved serine/threonine protein kinase that is activated by stress and genotoxic agents. JNK phosphorylates the amino terminal of all three Jun transcription factors and ATF-2 members of the AP-1 family. The activated transcription factors modulate gene expression to generate appropriate biological responses, including cell migration and cell death. When PC-3 cells were treated with varies dosages of γ-T3, a dosage- and time-dependent increase in JNK phosphorylation activities were detected (FIG. 12B). Meanwhile, phosphorylation of the JNK downstream effectors such as ATF-2 or c-jun were all upregulated by γ-T3, supporting that JNK signalling pathway was activated by the γ-T3.

As described above for the skin cancer cell lines, to further confirm the importance of JNK activation in γ-T3 induced apoptosis in PCa cells, it was investigated whether inactivation of JNK with a specific inhibitor, SP600125, could protect cells from γ-T3. As shown in FIG. 12A, co-treatment of γ-T3 together with 20 μM of SP600125, a dose that was previously determined to inhibit JNK activity in the same cell lines, decreased the percentage of apoptotic cells compared to that treated with γ-T3 alone, confirming that JNK activation may be required for γ-T3-induced apoptosis.

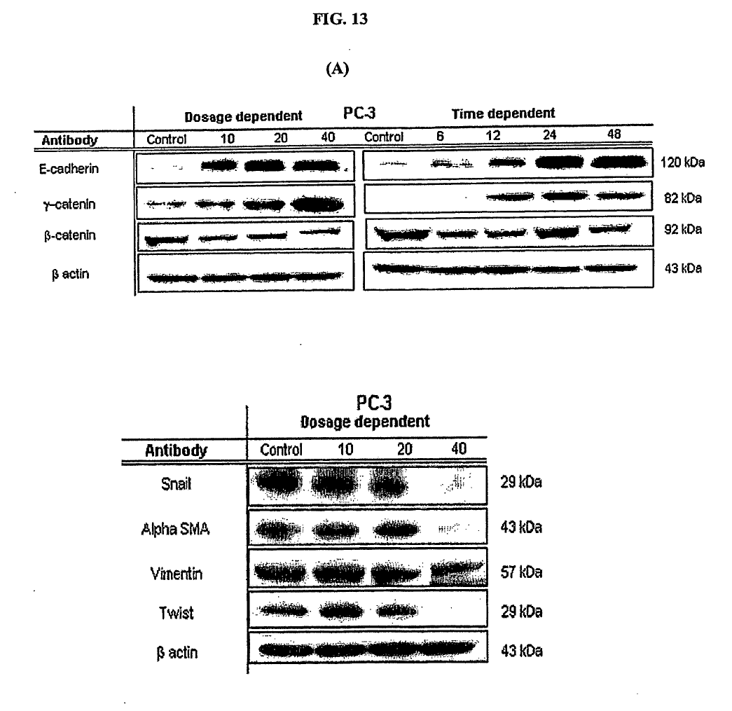

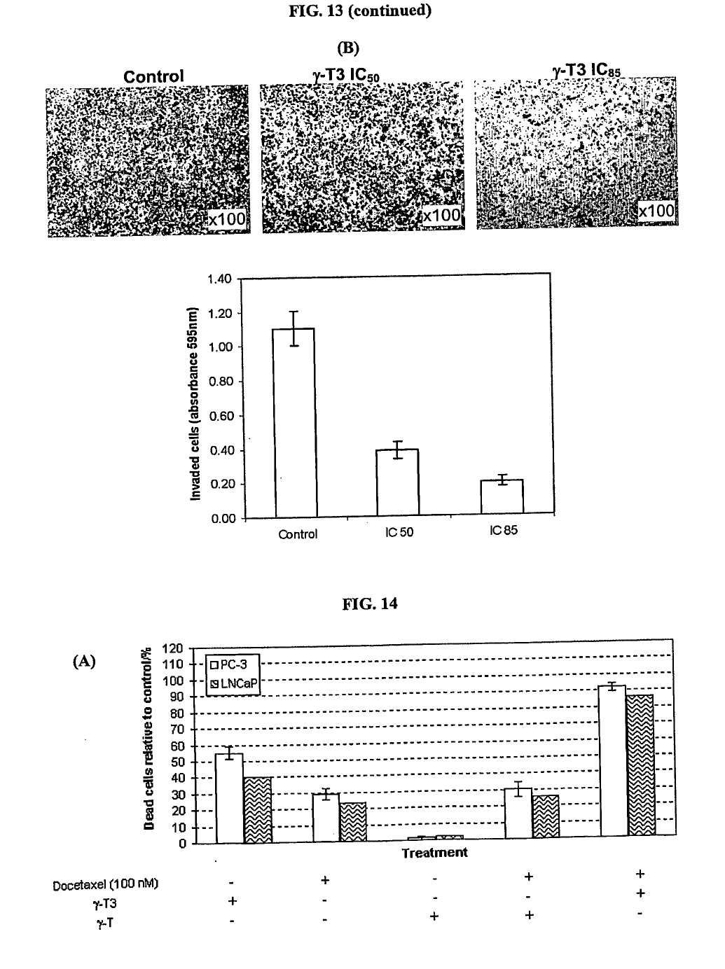

Effect of γ-T3 on inhibition of cell invasion—Although γ-T3 has been shown to have anti-proliferation effect on many cancers, it is not clear if it affects cancer metastasis. Therefore, it was examined whether γ-T3 could suppress the invasive ability of the prostate cancer cells. As shown in FIG. 13B, using matrigel-invasion assay, it was found that γ-T3 treated (IC₅₀) PC-3 cells for 24 hrs showed an at least 2.5-time lower invasion capability compared to the untreated control as evidenced by decreased in the number of cells invaded through the matrigel layer. This inhibitory effect on cell invasion was not the result of cell growth inhibition induced by γ-T3 as the number of viable cells added into the invasion chamber were the same. These results indicate that γ-T3 is able to inhibit the invasion ability of PCa cells, independent to their cytotoxic effects.

Down-regulation of E-cadherin expression is one of the most frequently reported characteristics of metastatic cancers. Restoration of E-cadherin expression in cancer cells leads to suppression of metastatic ability. In PCa, down-regulation of E-cadherin expression is correlated with high-grade tumours and poor prognosis, indicating their roles in PCa progression. Interestingly, it was found that E-cadherin and γ-catenin protein expression were up-regulated whereas E-cadherin's repressor (Snail) were down-regulated after treatment with γ-T3 (FIG. 13A), although the expression for β-catenin remained constant at all treatment dosages and time points. Owing to the deletion of the α-catenin in PC-3 cells, there was no expression detected. Separately, the mesenchymal markers (vimentin, α-SMA and twist) were all down-regulated after treatment with γ-T3 for 24 hrs.

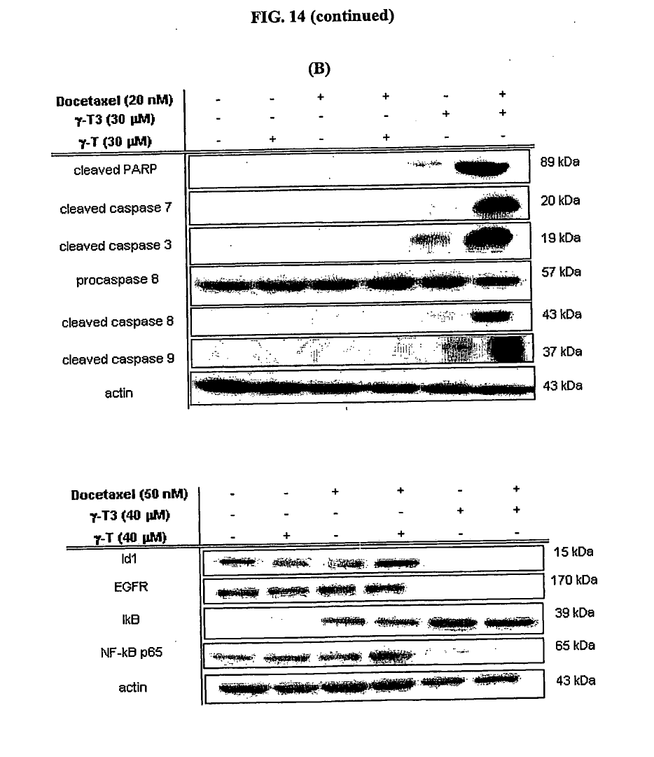

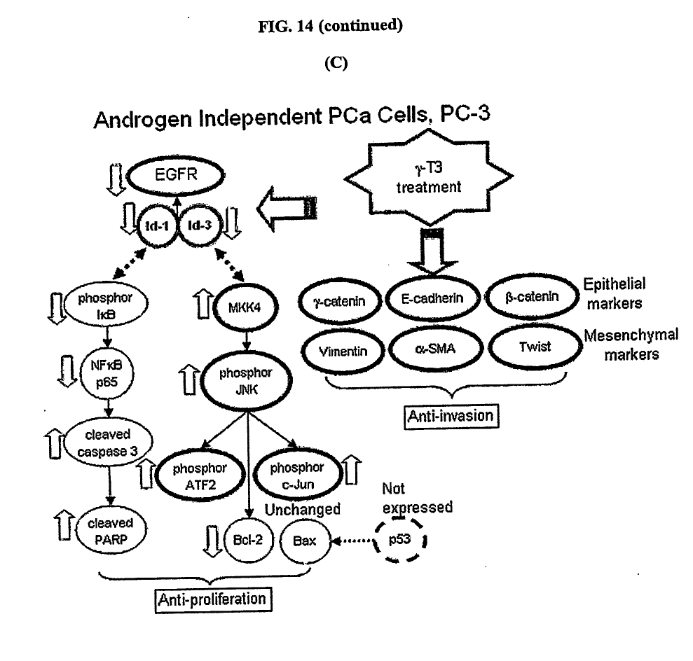

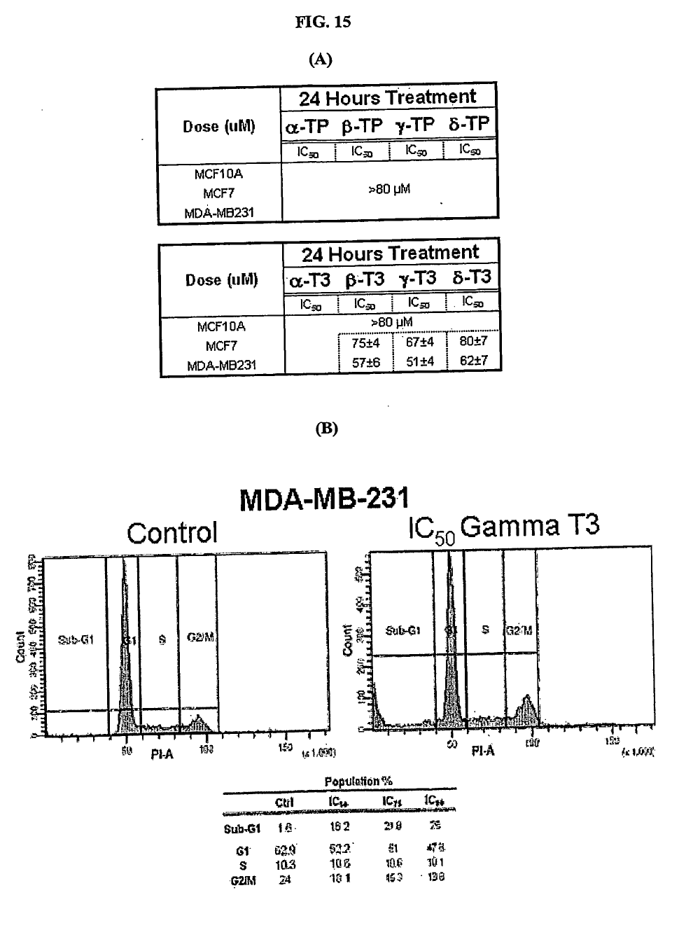

Effect of γ-T3 treatment on Docetaxel induced apoptosis—To test if γ-T3 can act synergistically with chemotherapeutic agent, the effect of γ-T3 alone or in combination with a anti-cancer agent, such as Docetaxel was compared. As shown in FIG. 14A, the percentage of apoptotic cells in PC-3 and LNCaP cell lines following co-treatment of Docetaxel with γ-T3 for 24 hrs was significantly higher than that treated with γ-T3 or Docetaxel alone. Using western blotting, it was further demonstrated that γ-T3 co-treatment with Docetaxel enhances cell apoptosis through activation of pro-apoptotic proteins (cleaved PARP, caspases 3, 7, 8, 9) and down-regulation of pro-survival proteins (Id-1, EGFR, iκB and NF-κB p65) (FIG. 14B). The level of apoptotic cells is in stark contrast to the γ-T co-treatment with Docetaxel. These results demonstrate that γ-T3 and Docetaxel have synergistic effect against prostate cancer cells.

The experiments referred to herein demonstrated for the first time that γ-T3 inhibit cell invasion by restoration of the E-cadherin, γ-catenin expression and suppression of mesenchymal markers. Together with the finding that γ-T3 enhanced the anti-cancer effect of Docetaxel, the experiments referred to herein provide strong evidences that γ-T3 can be developed as a safe and effective anti-cancer agent for the treatment of prostate cancer. The results suggest that γ- and δ-T3 possess tumor suppressing activities with different cell type specificity and potency.

In the experiments referred to herein it was demonstrated that γ-T3 suppressed constitutive NF-κB activity through inhibition of IκB kinase activation, leading to apoptosis in PCa cells. It was also demonstrated that γ-T3 induced NF-κB inactivation also downregulates the level of bcl-2 in a dosage-dependent and time-dependent fashion. Consequently, this induced apoptosis via activation of caspases 3, 7, 8, 9 and PARP. Consistent with previous results obtained with diverse cell lines differing in p53 status, the results showed that p53 is not required for γ-T3-induced apoptosis, since the p53-null cell lines (PC-3 and HL-60) are still responsive to γ-T3 treatment. It is worth noting that γ-T3 was previously demonstrated to abolish NF-κB activation induced by epidermal growth factor (EGF) and other pro-inflammatory cytokines. Although the molecular mechanism involved was not clear at that time, it was proposed that γ-T3 may act through a common step in the suppression of NF-κB. The experimental results referred to herein revealed that downregulation of EGF receptor (EGF-R) was correlated to γ-T3 induced NF-κB inactivation (FIG. 11B). This finding may explain why γ-T3 was able to suppress NF-κB activation by EGF treatment in KBM-5 cells. Interestingly, the androgen-independent prostate cancer cell line PC-3 was found to be more sensitive to γ-T3 treatment than the androgen-dependent LNCaP cells. PC-3 cells were found to have constitutive NF-kB activation and are in general more resistant to chemotherapeutic drugs-induced apoptosis than the LNCaP cells. Although the exact reason for this observation is unclear, but based on the fact that non-tumorigenic prostate epithelial cells are highly resistant to γ-T3 as well, it is possible that γ-T3 may preferentially target the cells with higher malignant phenotype.

It is believed that one possible mechanism by which γ-T3 could mediate its effects on the NF-κB pathway is through the suppression of Id-1 and EGF-R. It was previously demonstrated that ectopic Id-1 expression in LNCaP cells resulted in increase of NF-κB transactivation activity and nuclear translocation of the p65 and p50 proteins, which was accompanied by up-regulation of their downstream effectors Bcl-xL and ICAM-1. In addition, inactivation of Id-1 by its anti-sense oligonucleotide and retroviral construct in DU145 cells resulted in the decrease of nuclear level of p65 and p50 proteins, which was associated with increased sensitivity to TNF-induced apoptosis. Considering these findings, the results referred to herein strongly suggest that Id gene family may be one of the upstream regulators of NF-κB that is targeted directly by γ-T3, and inhibition of NF-κB signalling pathway may be responsible for γ-T3 induced anti-proliferation.

It was also shown herein that c-Jun N-terminal kinase participates in γ-T3 induced apoptosis. When PCa cells were treated with γ-T3, a series of molecules associated with JNK pathway, such as c-Jun and ATF-2 (FIG. 12A), were activated simultaneously. Meanwhile, it was demonstrated that treatment of JNK inhibitor (SP600125) protects the PCa cells from γ-T3 induced apoptosis (FIG. 12A). This further confirms the involvement of JNK pathway in γ-T3 induced apoptosis in PCa cells. Worth noting is that, the JNK pathway is also known to be involved in cell apoptosis induced by the chemotherapeutic drug, Docetaxel. Taking these findings into consideration, it was therefore questioned whether γ-T3 possesses synergistic interaction with Docetaxel as a result of activation of JNK pathway. To this end, the anti-proliferation capability of Docetaxel treatment alone, and co-treatment with γ-T3 was compared. Remarkably, it was found that combined treatment of Docetaxel and γ-T3, but not γ-T, resulted in higher proportion of apoptotic cells (FIG. 14A). This finding indicates a synergistic role of γ-T3 with the chemotherapeutic agent.

Furthermore, it was demonstrated herein that restoration of E-cadherin and γ-catenin expression, together with suppression of snail, α-SMA, vimentin and twist, probably account for γ-T3's inhibitory effect on PCa cell invasion capability. Although the anti-proliferation effect of γ-T3 has been reported in several cancer types, the results referred to herein provide first evidence to suggest that it can also be a potential agent for suppression of cancer invasion. Down-regulation of E-cadherin and up-regulation of mesenchymal markers (α-SMA, vimentin and twist) are some of the most frequently reported phenomena in metastatic cancers. It is suggested that loss of E-cadherin expression is able to promote epithelial-mesenchymal transition (EMT), which plays a key role in the progression of cancer cells to metastatic stage. Although the precise mechanism responsible for E-cadherin inactivation in cancer cells is not clear, alterations at transcriptional level due to its repressor Snail seem to be one of the mechanisms responsible for its decreased expression in several cancer types. Due to the results from the experiments referred to herein, it was found that the γ-T3-treated PCa cells showed increased E-cadherin expression (FIG. 13A), which was associated with reduced Snail protein expression and invasion ability (FIG. 13B). Catenins (α, γ), a family of cytoplasmic cadherin binding proteins, link E-cadherin to the actin cytoskeleton and are thought to be essential for normal E-cadherin function. It was found that γ-T3 only up-regulated the expression of E-cadherin and γ-catenin, but not α-catenin. The expression for β-catenin remains static, similar to previous experiments using garlic derivatives. Although PC-3 cells do not express α-catenin, a key molecule for functional E-cadherin expression, γ-T3 might restore the function of E-cadherin through other molecules such as vinculin, which has been reported to play a role in the establishment of the E-cadherin-based cell adhesion complex. Taken together, the results referred to herein suggest that γ-T3 can suppress cancer metastasis through induction of mesenchymal-to-epithelial transition (MET).

As summarized in FIG. 14C, the results referred to herein demonstrate that γ-T3 is a potent and specific inhibitor of PCa cell proliferation and invasion which acts through multiple molecular pathways. Since no side effect can be observed after long term intake of natural T3 extract (LD₅₀≧2000 mg/kg, data not shown), γ-T3 may be used alone or in combination with chemotherapy for treating advanced stage PCa.

3. The following experiments provide explanations as to the anti-proliferative effect of gamma-tocotrienol comprising compositions on breast cancer (BCa) cells and the underlying molecular pathways responsible for its activity. The results showed that treatment of breast cancer cells with gamma-tocotrienol comprising compositions resulted in induction of apoptosis as evidenced by activation of pro-caspases, accumulation of sub-G1 cells and DNA fragmentation. Examination of the pro-survival genes revealed that the gamma-tocotrienol-induced cell death was associated with suppression of Id1 and NF-κB through modulation of their upstream regulators (Src, Smad1/5/8, Fak and LOX). Meanwhile, gamma-tocotrienol treatment also resulted in the induction of JNK signalling pathway and inhibition of JNK activity by specific inhibitor partially blocked the effect of gamma-tocotrienol. Furthermore, a synergistic effect was observed when cells were co-treated with gamma-tocotrienol and a chemotherapeutic agent, such as Docetaxel. Interestingly, in cells that treated with gamma-tocotrienol, alpha-tocopherol or β-aminoproprionitrile were found to partially restore Id1 expression. Meanwhile, this restoration of Id1 was found to protect the cells from gamma-tocotrienol induced apoptosis. The results suggested that the anti-proliferative and chemosensitization effect of gamma-tocotrienol on breast cancer cells is mediated through downregulation of Id1 protein.

3.1 Materials and Experimental Conditions

3.1.1 Breast Cancer cell lines, cell culture conditions and chemicals—The human estrogen-dependent BCa cells (MCF-7), human estrogen-independent BCa cells (MDA-MB-231), androgen-independent prostate cancer cells (PC-3) (ATCC, Rockville, Md.) were maintained in their respective medium recommended by ATCC (Invitrogen, Carlsbad, Calif.) supplemented with 2 mmol/l L-glutamine, 10% fetal calf serum (FCS) and 2% penicillin streptomycin at 37° C. in 5% CO₂. The immortalized human non-tumorigenic breast epithelial cell line (MCF-10A) (ATCC, Rockville, Md.) was maintained in MEBM, which is supplied as part of the MEGM Bullet Kit available from Clonetics Corporation. To make the complete growth medium, the following components were added into the base medium: All MEGM SingleQuot additives that are supplied with the kit except the GA-1000 (BPE 13 mg/ml, 2 ml; hydrocortisone 0.5 mg/ml, 0.5 ml; hEGF 10 ug/ml, 0.5 ml; insulin 5 mg/ml, 0.5 ml); 100 ng/ml cholera toxin. The stable Si-Id1 PC-3 cell line (Id1 knockdown model) was contributed by Prof Y C Wong (HKU) based on previous protocol. Docetaxel (Calbiochem, Darmstadt, Germany), JNK inhibitor SP600125, Erk inhibitor U0126 (Sigma-Aldrich, St. Louis USA) and β-aminoproprionitrile (APN) (TCI, Japan) were dissolved in dimethylsulfoxide (DMSO). The treatment solutions were diluted in culture medium to obtain the desired concentrations.

3.1.2 Generation of Id1 transfectant—MDA-MB-231 cells (1×10⁵ cells/well) were plated into 12-well culture plates and allowed to grow for 24 hrs. pc-Id1 or pcDNA (a gift from Prof MT Ling, IHBI) was transfected into the cells using Fugene 6 reagent for 24 hrs before gamma-T3 treatment. 24 hrs later, the cells were either assayed for MTT cell viability or lysed for western blotting.

3.1.3 For the following experiments the same tocotrienol and tocopherol isomers have been used as described above under 1.1.2.

3.1.4 Cell viability and time course experiments were carried out as described above under item 1.1.3 with the following difference. For inhibitors study, cells were pre-treated with inhibitors (U0126, PD98059 and APN) at targeted dosage for 8 hrs prior to the addition of vitamin-E isomers.

3.1.5 DNA fragmentation assay—After 24 hrs incubation with gamma-T3, 3×10⁶ MDA-MB-231 cells were harvested and suspended in lysis buffer [5 mM Tris-HCl (pH 8.0), 20 mM EDTA, and 0.5% (v/v) Triton X-100] for 60 min on ice. Samples were centrifuged, the supernatants were removed and incubated with 5 μl RNase A (10 μg/ml) at 37° C. for 40 min, and 1 ml of anhydrous ethanol was added. Tubes were placed at 20° C. for 20 min and then centrifuged to pellet the DNA. DNA samples were analyzed by electrophoresis at 80 V for 3 hrs on a 2% agarose gel containing ethidium bromide (0.2 μg/ml) and visualized under UV illumination.

3.1.6 Terminal deoxynucleotidyltransferase-mediated deoxyuridine triphosphate nick end-labelling (TUNEL) assay—DNA strand breaks during apoptosis was examined using in situ cell death detection reagent (Roche Applied Science). Briefly, 1×10⁶ cells were pretreated with gamma-T3 for 24 hrs. Thereafter, cells were incubated with reaction mixture for 60 min at 37° C. Stained cells were analyzed and captured by fluorescence microscope on glass slide.

3.1.7 Flow cytometry was carried out as described above under item 1.1.3. Matrigel-invasion assay was carried out as described above under item 1.1.4. Western blotting was carried out as described above under item 1.1.5.

3.1.8 Id-1 RT-PCR—Total RNA was isolated using Trizol® reagent according to the manufacturer's protocol (Invitrogen). cDNA was synthesized using the SuperScript™ First Strand Synthesis System (Invitrogen) and was then amplified by PCR with Id-1 specific primers (forward primer, Id1-S, 5′-CTC CAG CAC GTC ATC GAC TA-3′ and reverse primer, Id1-AS,5′-AAC GCA TGC CGC CTC-3′). PCR cycling protocol was as follows: 30 cycles of 10 min at 95° C., 30 s at 95° C., 30 s at 55° C., 1 min at 72° C. and 10 min at 72° C. Glyceraldehyde 3-phosphate dehydrogenase was amplified as an internal control. The PCR products were electrophoresed on a 2% agarose gel and analysed using a gel documentation system.

3.2 Results—Anti-Proliferation Effect of Vitamin-E Isomers on Breast Cancer (BCa) Cells

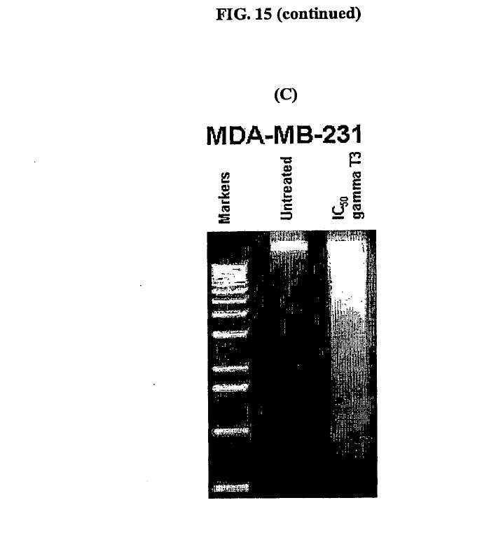

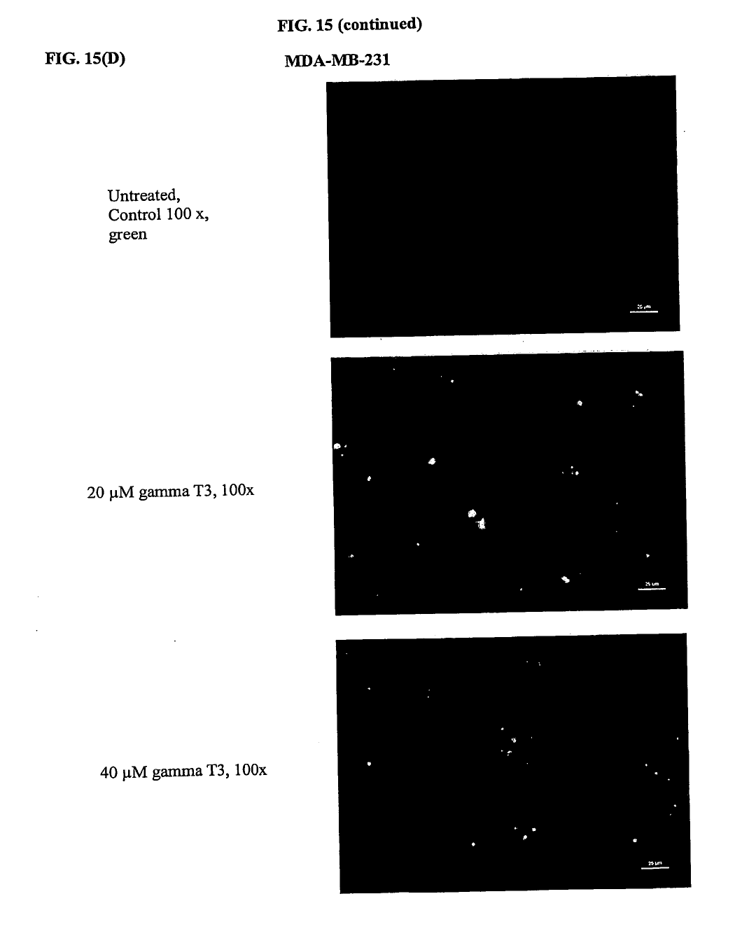

BCa cells were treated with vitamin-E isomers for 24-hr at increasing dosage (low: 20, medium: 40 μM and high: 80 μM). The results showed that vitamin-E isomers did not affect the proliferation rate of normal breast epithelial cells (MCF-10A), but significantly suppressed the proliferation of MCF-7 and MDA-MB-231 (FIG. 15A). Surprisingly, MDA-MB-231 cells were more sensitive to the growth inhibition of the vitamin-E isomers than MCF-7 cells. The inhibition of cell proliferation was stronger for T3 isomers in MDA-MB-231, particularly for gamma-T3, which showed a dose-dependent inhibition. Based on the IC₅₀ values in MDA-MB-231 cells incubated with various isomers for 24-hr, the order of inhibitory effect is gamma-T3>beta-T3>delta-T3. Since MDA-MB-231 cells are considered to be more invasive and resistant to chemotherapeutic agents when compared to MCF-7 cells, for the subsequent experiments, it was decided to investigate the effect of gamma-T3 on MDA-MB-231.

To study the mechanism responsible for gamma-T3-induced growth inhibition, cell cycle distribution and genomic DNA fragmentation of the cells with or without gamma-T3 treatment for 24 hrs were analyzed by flow cytometry, gel electropheresis and TUNEL assays. Consequently, treatment of cells with gamma-T3 (IC₅₀₋₉₀) resulted in an induction of sub-G1 cell population (FIG. 15B) and DNA fragmentations (FIG. 15C-D), indicating the presence of apoptotic cells after the treatment. The proportion of apoptotic cells (sub-G1 fraction) increased in a dose-dependent manner.

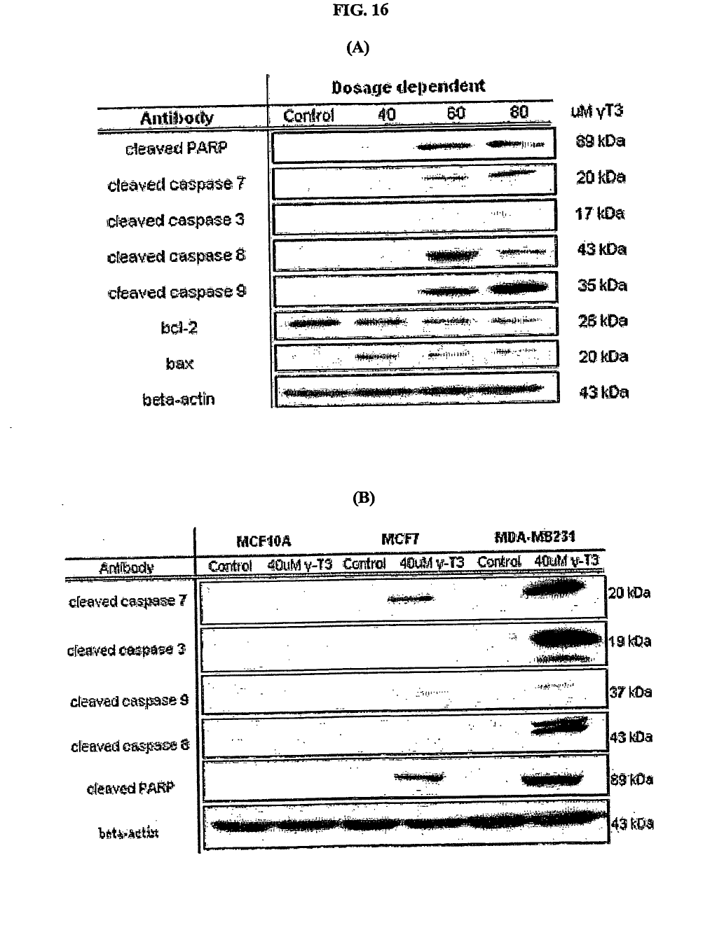

To study further the mechanism of gamma-T3 induced apoptosis, it was at first investigated if the programmed cell death in MDA-MB-231 cells is caspase-dependent. As shown in FIG. 16A activation of procaspase 3, 7, 8, 9 as well as PARP, as evidenced from the appearance of the cleaved products, were observed in MDA-MB-231 cells treated with different gamma-T3 dosage for 24 hrs. Downregulation of bcl-2 was also detected after the treatment, together with upregulation of bax expression (FIG. 16A). Meanwhile, these gamma-T3-mediated activations of the pro-apoptotic proteins as well as the change of bcl-2/Bax ratio were in a dose-dependent manner (FIG. 16A). In addition, activation of these pro-apoptotic genes by gamma-T3 treatment (FIG. 16B) was only observed in MDA-MB-231 and MCF-7 cells, but not in MCF-10A cells, indicating that gamma-T3 specifically induced apoptosis in BCa cells.

3.3 Gamma-T3 Downregulated the Pro-Survival Signalling Pathways in BCa Cells

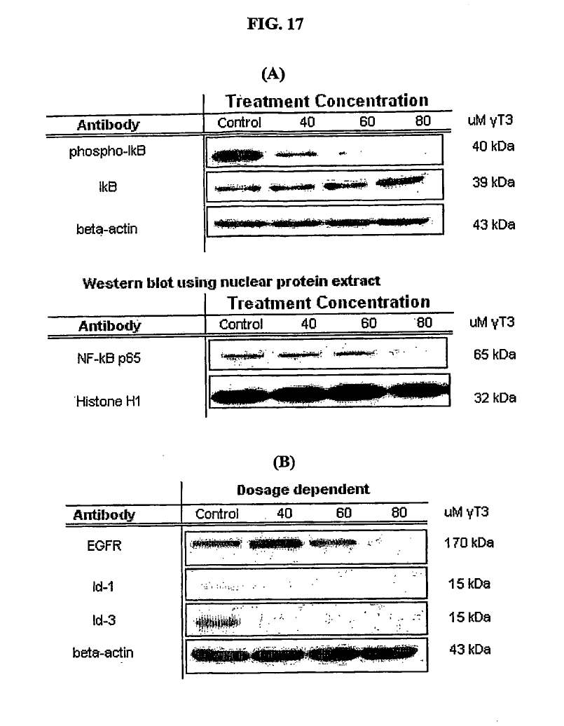

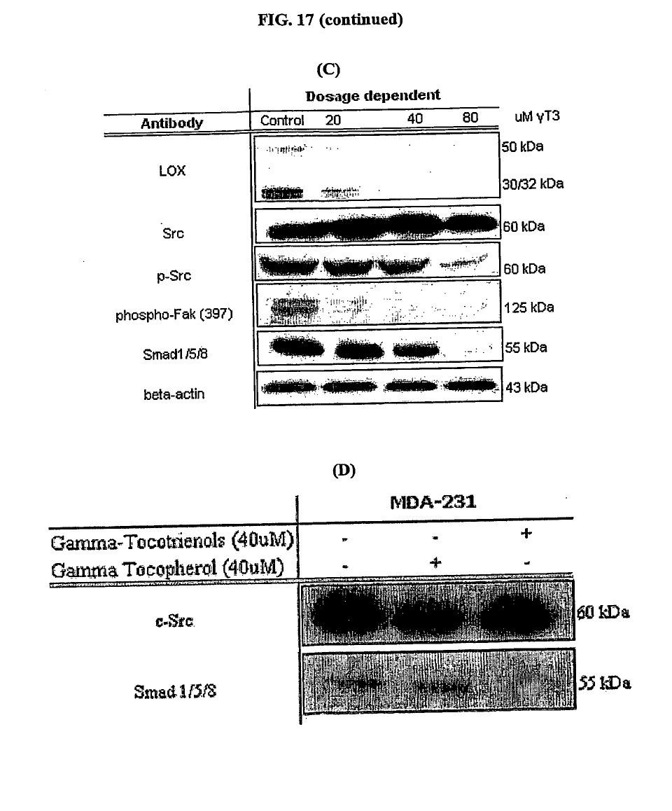

Because NF-κB was reported to be constitutively activated in MDA-MB-231 cells, the possibility that gamma-T3 induced cell apoptosis attributable to the suppression of NF-κB activation was considered. The NF-κB activities of MDA-MB-231 treated with gamma-T3 at different dosages were measured by examining the nuclear translocation of NF-κB subunit p65. As illustrated in FIG. 17A, gamma-T3 treatment suppressed nuclear level of NF-κB p65 in a dose-dependent manner. The effect of gamma-T3 on NF-κB signalling was further explored by examining the expression of other upstream regulators, such as p-IκBα/β and IκBα/β. In gamma-T3 treated MDA-MB-231 cells, a dose-dependent decrease in the level of the phosphorylated IκBα/β were observed (FIG. 17A). This is associated with the increase in the level of IκBα/β, as well as an inhibition of NF-κB p65 nuclear translocation. These results indicate that γ-T3 suppressed NF-κB activity through the dephosphorylation and accumulation of IκBα/β.

3.4 Gamma-T3 Downregulated the Id1 Signalling Pathway and its Upstream Regulator Proteins in BCa Cells

Surprisingly, it was found that gamma-T3 treatment also downregulated a number of the key proteins that are involved in the development and progression of BCa. As shown in FIG. 17B, Id1 and Id3 expressions were significantly suppressed to almost undetectable level by treatment with increasing dosages of gamma-T3. Similar effect on EGF-R protein level was observed. Since EGF-R and Id protein family are essential for BCa cell growth and survival, their downregulation may be associated with the gamma-T3 induced growth arrest and apoptosis.

Because Id1 transcript and protein levels were previously shown to be regulated directly or indirectly by the Src, Smad1/5, LOX and Fak signalling pathways in BCa cells, it was further examined the effect of gamma-T3 on the upstream regulators of Id1 in BCa cells. The results showed that the Src phosphorylation, as well as the protein level of Smad1/5/8, LOX and activated Fak were repressed in a dose dependent manner by gamma-T3 treatment (FIG. 17C). Meanwhile, immunoprecipitation assay revealed using anti-Src antibody revealed a decrease of interaction between Src and Smad1/5/8, which is likely due to the suppression of Smad1/5/8 protein level by gamma-T3 (FIG. 17D). This possibly led to decreased binding of Src-Smad complex to Src-responsive region of the Id-1 promoter, resulting in the observed suppression of Id1 protein expression by gamma-T3.

3.5 Gamma-T3 Activated the Pro-Apoptotic Signalling Pathways in BCa Cells

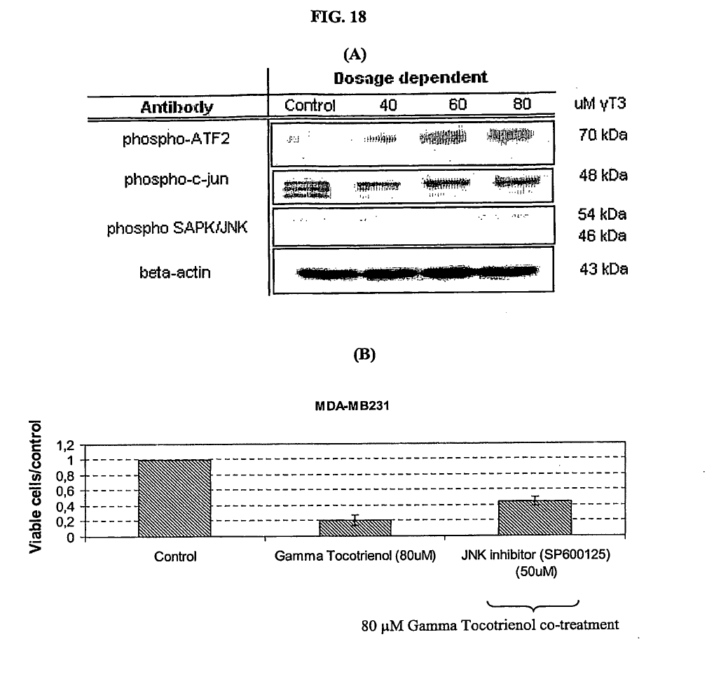

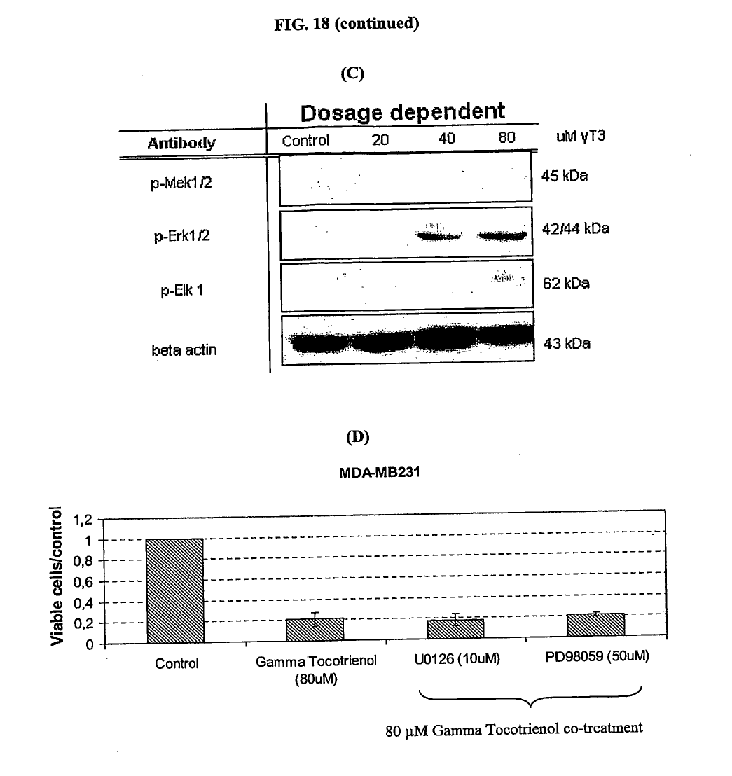

The c-Jun N-terminal kinase (JNK) is an evolutionarily conserved serine/threonine protein kinase that is activated by stress and genotoxic agents. JNK phosphorylates the amino terminal of all three Jun transcription factors and ATF-2 members of the AP-1 family. The activated transcription factors modulate gene expression to generate appropriate biological responses, including cell migration and cell death. When MDA-MB-231 cells were treated with varies dosages of gamma-T3, a dose-dependent increase in JNK phosphorylation activities were detected (FIG. 18A). Meanwhile, phosphorylation of the JNK downstream effectors such as ATF-2 or c-jun were all upregulated by gamma-T3, supporting that JNK signalling pathway was activated by gamma-T3.

To study the importance of JNK activation in gamma-T3 induced apoptosis in BCa cells, it was investigated whether inactivation of JNK with a specific inhibitor, SP600125, could protect cells from gamma-T3. As shown in FIG. 18B, co-treatment of gamma-T3 together with 20 μM of SP600125 was found to increase the percentage of viable cells when compared to that treated with gamma-T3 alone, confirming that JNK activation may be required for gamma-T3 induced apoptosis.

3.6 Activation of MAPK/ERK Pathway was not Associated with Gamma-T3 Induced Apoptosis in BCa Cells

The MAPK/ERK kinase is one of the intracellular signalling pathways which is activated by different stimuli, including growth factors, cytokines and carcinogens. Although mitogen-activated protein kinase (MAPK/ERK) pathway was found to be activated by gamma-T3 in MDA-MB-231, as evident by phosphorylation of Erk1/2, Mek1/2 and Elk1 (FIG. 18C), their activation may not be directly required for gamma-T3 induced apoptosis because inactivation of MAPK by specific inhibitors, U0126/PD98059, were not able to restore cancer cell viability after gamma-T3 treatment (FIG. 18D).

3.7 Effect of Gamma-T3 on Inhibition of BCa Cell Invasion

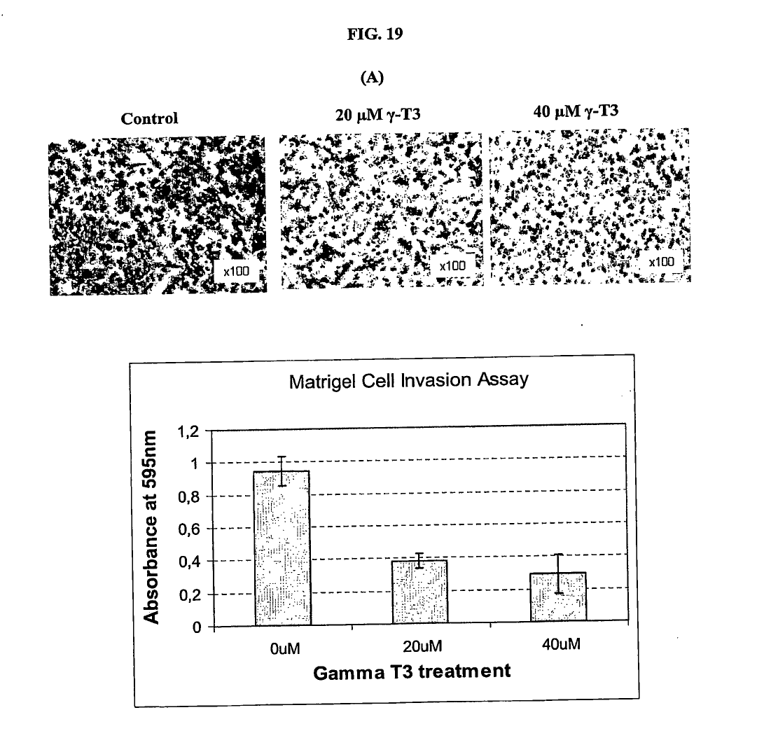

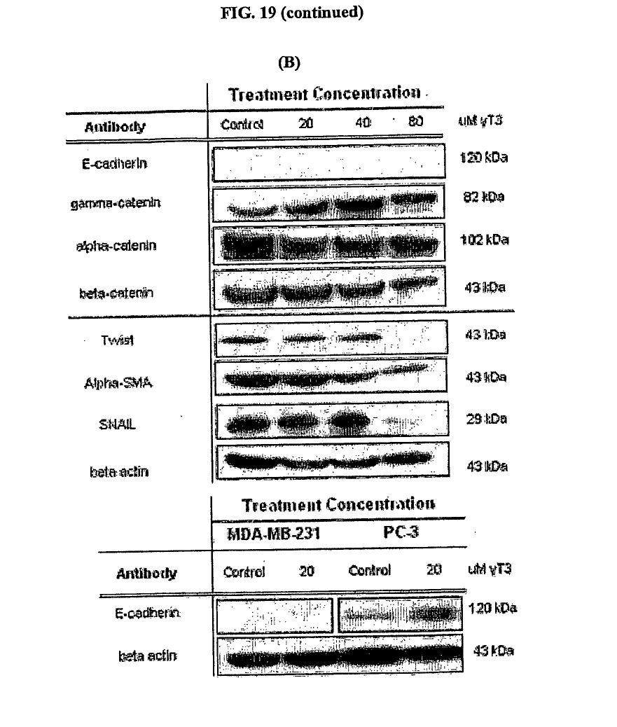

Although gamma-T3 has been shown to have anti-proliferation effect on many cancers, it is not clear if it affects BCa metastasis. Therefore, it was examined whether gamma-T3 could suppress the invasive ability of the BCa cells. As shown in FIG. 19A, using matrigel-invasion assay, it was found that gamma-T3 treated MDA-MB-231 cells for 24 hrs showed an at least 2-time lower invasion capability compared to the untreated control, as evidenced by the decreased in the number of cells invaded through the matrigel layer. This inhibitory effect on cell invasion was not the result of cell growth inhibition induced by gamma-T3 as the number of viable cells added into the invasion chamber was the same. These results indicate that gamma-T3 is able to inhibit the invasion ability of BCa cells, independent to their cytotoxic effects.

Down-regulation of E-cadherin expression is one of the most frequently reported characteristics of metastatic cancers. Restoration of E-cadherin expression in cancer cells leads to suppression of metastatic ability. In BCa, down-regulation of E-cadherin expression is correlated with high-grade tumours and poor prognosis, indicating their roles in BCa progression. It was so far not possible to detect MDA-MB-231 as it is an E-cadherin-negative human BCa cell line. Meanwhile, gamma-T3 treatment failed to affect α- and β-catenin protein expression but enhanced the γ-catenin expression. The expression of Snail and Twist, the two E-cadherin repressors were both downregulated after treatment with γ-T3 (FIG. 19B). In addition, the mesenchymal markers α-SMA was down-regulated after treatment with gamma-T3 for 24 hours (FIG. 19B), indicating that gamma-T3 can suppress BCa invasion through inhibition of epithelial to mesenchyme transition (EMT).

3.8 Effect of Gamma-T3 Treatment on Docetaxel Induced Apoptosis

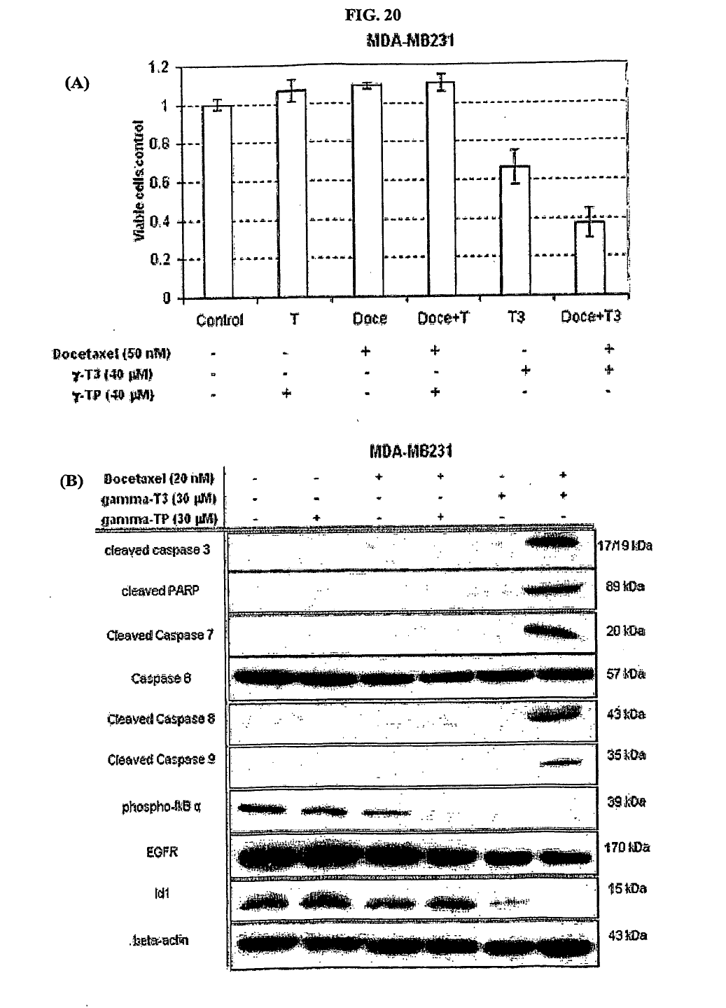

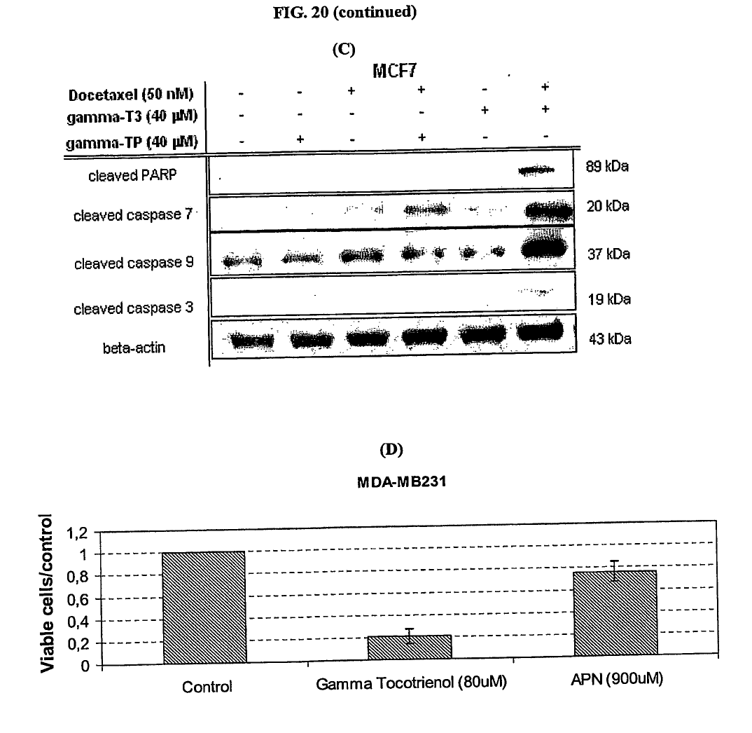

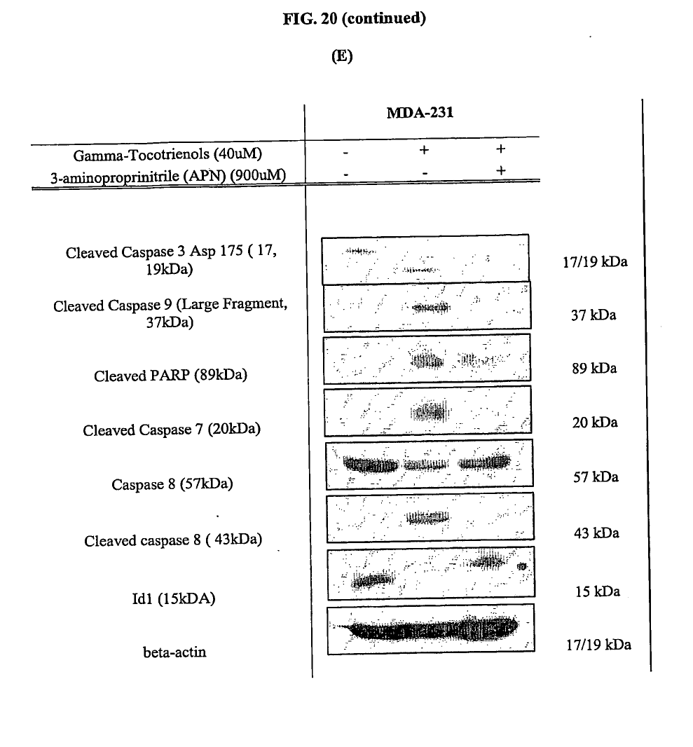

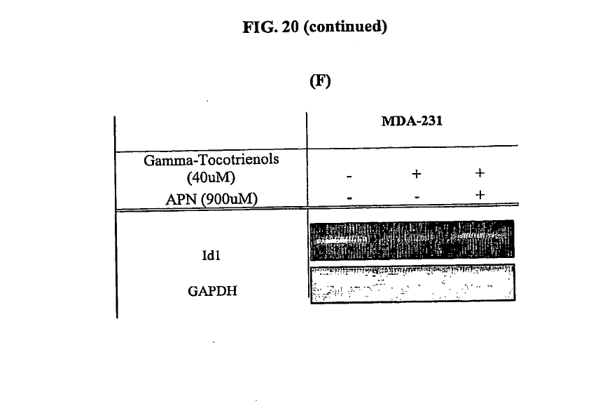

Many of the natural products, such as aged garlic extract or resveratrol which are extracted from fruit or plant have been shown to have anti-cancer effect. Previous studies have shown that many of these natural products increased the sensitivity of cancer cells to chemotherapy and enhanced the effectiveness of radiation treatment against prostate tumor. To test if gamma-T3 can act synergistically with chemotherapeutic agent, the effect of gamma-T3 alone or in combination with Docetaxel was compared. As shown in FIG. 20A, the percentage of apoptotic cells in MDA-MB-231 cell line following co-treatment of Docetaxel with gamma-T3 for 24 hrs was significantly higher than that treated with gamma-T3 or Docetaxel alone. Using Western blotting, we further demonstrated that gamma-T3 co-treatment with Docetaxel enhances cell apoptosis through activation of pro-apoptotic proteins (cleaved PARP, caspases 3, 7, 8, 9) and down-regulation of pro-survival proteins (Id-1, EGFR) (FIG. 20B). Similar effect was also observed in MCF-7 cells (FIG. 20C), suggesting that gamma-T3 and Docetaxel may have synergistic effect against BCa cells.

3.9 β-Aminopropionitrile (APN) Attenuated Gamma-T3 Induced Apoptosis

Co-treatment of gamma-T3 with β-aminopropionitrile (APN; a non-specific inhibitor of LOX) almost completely restored the expression of Id1 and at the same time inhibited the gamma-T3-induced caspase-dependent apoptosis, as evident from the cell proliferation and Western blotting analysis (FIGS. 20D&E). However, the marginal decrease in the levels of PARP cleavage as seen with gamma-T3 and gamma-T3-APN co-treatment suggested an induction of caspase-independent apoptosis. These findings are unexpected and thus suggest involvement of other mechanisms leading to Id1 induction during gamma-T3 and APN co-treatment. FIG. 20F summaries the anti-cancer pathway for gamma tocotrienol in breast cancer cells.

4. In the following experiments the gamma-tocotrienol (γ-T3) in vivo antitumor effect for prostate cancer (PCa) tumors was investigated together with its pharmacokinetic, tissue distribution and synergistic interaction with Docetaxel. Briefly, after intra-peritoneal injection, γ-T3 rapidly disappears from serum and selectively deposit in PCa tumors. Short term administration of γ-T3 resulted in significant shrinkage of the tumors. Meanwhile, further inhibition of the tumor growth was achieved by combined treatment of γ-T3 and Docetaxel. The antitumor effect of γ-T3 was associated with the decrease in expression of cell proliferation markers and increase in the rate of cancer cell apoptosis. The results demonstrated the in vivo antitumor of γ-T3 against PCa tumors.

4.1 Materials and Experimental Conditions

4.1.1 Human prostate cancer cell line, PC-3, was obtained from ATCC and was grown in RPMI 1640 (Invitrogen, Carlsbad, Calif., USA) supplemented with 1% penicillin streptomycin and 5% fetal bovine serum (FBS) (PAA Laboratories GmbH, Pasching, Austria) in humidified 95% air, 5% CO2 at 37° C. Docetaxel (Calbiochem, San Diego, Calif., USA), was dissolved in dimethyl sulphoxide (Sigma Aldrich, St Louis, Mo., USA). Solvents such as heptane and ethyl acetate were bought from Tedia Company Inc. (Fairfield, Ohio, USA). D-luciferin, Butylated hydroxytoluene (BHT) and 10% neutral buffered formalin were obtained from Sigma Aldrich (St Louis, Mo., USA).

4.1.2 For the following experiments the same tocotrienol and tocopherol isomers have been used as described above under 1.1.2.

4.1.3 Establishment of the PC-3 prostate cancer xenograft model—Bioluminescent PC-3-Luc human prostate cancer cell line were generated according to known methods. Briefly, cDNA encoding the luciferase gene was cloned into the pLenti-6/V5. The construct was co-transfected with the packaging mix into HEK293 and lentivirus were collected and used for infecting PC-3 cells. Transfectants were obtained as a pool (PC-3-Luc) by selection with 10 μg/mL of Blasticidine for 1 week. The animal experimental protocol was approved by NACLAR (National Advisory Committee for Laboratory Animal Research) Guideline of Singapore for proper and humane use of animals. Male BALB/c athymic nude mice (4-5 weeks old, 18-22 g) were purchased from The Jackson Laboratory (Bar Harbor, Me., USA). Mice were housed in Department 1, Biological Resource Centre (Biopolis, Singapore) under standard condition (20.8±2° C., 55±1% relative humidity, 12 h light/dark cycle) with rodent diet (Harlan Laboratories, Inc., Indianapolis, Ind.) and chlorinated reverse osmosis water supplied in pathogen free environment. Briefly, 1×10⁶ PC-3-Luc cells in 100 μl serum free RPMI 1640 were injected subcutaneously into the flank of nude mice using a 1-ml syringe with 26-gauge needle (Becton Dickinson, Franklin Lakes, N.J., USA). All surgical operations were performed under aseptic conditions.

Nude mice bearing similar tumor sizes of about 100 mm³ (after 2 weeks inoculation) were selected and randomly divided into three groups (n=5 per group); control (DMSO as vehicle), γ-T3 (50 mg/kg/d) and combination treatment of γ-T3 and Docetaxel (50 mg of γ-T3/kg/d and 7.5 mg of Docetaxel/kg/wk). The mice were weighed as daily basic and the tumors were measured using a Digital Carbon Fiber Caliper (Fisher scientific, Pittsburgh, Pa.) at the same time. The tumor volume was calculated as 4/3*π*(mean diameter/2)³. The mice were dosed 5 times a week for 2 weeks. After 10 days of treatment, the mice were euthanized by CO₂ inhalation. Blood samples were collected through cardiac bleeding using 25-gauge needle. Blood samples were incubated at room temperature for 30 min, followed by centrifugation at 4400 rpm, 4° C. for 30 min. Serum, as the supernatant, was separated from plasma and stored at −80° C. Tumor, liver, kidney, spleen, lung and heart were harvested. Part of the tumors was fixed in 10% neutral buffered formalin solution. The remaining of the tumors and all the isolated organs were immediately immersed in liquid nitrogen and store at −80° C.

4.1.4 Pharmacokinetics of γ-Tocotrienol in Mice—C57BL/6 black mice were purchased from The Jackson Laboratory (Bar Harbor, Me., USA). Forty 5-week old mice were given a single dose i.p injection containing 1 mg of γ-T3. Five mice were sacrificed at different time points (10 min, 30 min, 1 h, 3 h, 6 h, 24 h, 48 h and 72 h). Blood samples were collected through cardiac bleeding. To isolate the serum, blood samples were incubated at room temperature for 30 min, followed by centrifugation at 4400 rpm, 4° C. for 30 min. γ-T3 concentration in serum was analyzed using HPLC method below.

4.1.5 Single Acute Toxicity Test—The maximum tolerated dose (MTD) was determined by increasing doses on different groups of mice until the highest dose without any mortality is found. Briefly, ninety C57BU6 black mice (ten for each group) received single dose i.p injection containing 1, 2, 4, 8, 12, 16, 20, 30 and 40 mg of γ-T3 in 100 μl injection volume. The weight and survival of mice were observed for 30 days, followed by euthanized by CO₂ inhalation.

4.1.6 γ-Tocotrienol Extraction from Serums, Tumors and Organs—Serums were thawed and sonicated in an ultrasonic bath (Lab Companion, Vernon Hills, Ill., USA) for 5 min, followed by vortexing for 10 s. 100 μl of serum was transferred into IWAKI Pyrex glass tube (Jawa Tengah, Indonesia) containing 900 μl of water. For the tumors and organs preparation, the tissues were homogenized in 1 ml of water using borosilicate glass homogenizer (Fisher scientific, Pittsburgh, Pa.), followed by transferring to Pyrex glass tube. 5 μl of δ-T3 with purity 99% (100 mg of δ-T3 dissolved in 1 ml of ethanol) was used as an internal standard solution and was spiked into the mixture. The tube was vortexed for 10 s and sonicated for 2 min. 4 ml of the butylated hydroxytoluene (BHT) solution (5 mg of BHT in 100 ml of heptane) was added into the tube to minimize the oxidation of target analytes. Liquid-liquid extraction was performed by vortexing vigorously for 10 s. After liquid-liquid extraction, the tubes were centrifuged at 4000 rpm for 5 min in Heraeus Multifuge 3-SR Centrifuge (Newport Pagnell, Buckinghamshire, UK). 3.9 ml of the organic layer was transferred into another Pyrex tube. The extraction was repeated and second organic layer was took out and pooled together with the first layer. The organic solution was evaporated using Buchi rotavapor R-205 (Flawil, Switzerland), and the dried residue was reconstituted in 1.5 ml of heptane, filtered, followed by HPLC analysis.

4.1.7 Determination of γ-Tocotrienol level by high performance liquid chromatography—A normal phase of HPLC method was performed as a modification of procedures known in the art. 10 μl of sample was injected into Agilent 1100 series HPLC system (Agilent, Santa Clara, Calif., USA). The chromatographic separation was carried out by a Zorbax Silica 60 (5 μm, 250×4 mm internal diameter (i.d.)) analytical column. The mobile phase used was a mixture of heptane/ethyl acetate (90:10, v/v) at a flow rate of 1.0 ml/min. The absorbance of γ-T3 was monitored with a diode array detector set at an excitation wavelength of 290 nm and emission wavelength of 360 nm.

4.1.8 Serum-based toxicity assay—Ten C57BL/6 black mice were given 5 dose intraperitoneal (i.p) injections per week containing 1 mg of γ-tocotrienol or DMSO blank. Mice were sacrificed by cardiac bleeding and the serum was extracted by method described above. Serum level of the biomarkers albumin, creatine, alanine transaminase ALT, aspartate aminotransferase AST, urea and alkaline phosphatase ALP were then measured by the colorimetric-based detection kits purchased from RANDOX laboratories Ltd. (Crumlin, United Kingdom).

4.1.9 Immunohistochemistry—Tumor, liver, kidney, spleen, lung and heart of mice were fixed in 10% neutral buffered formalin for 12 h. After fixation, the tissue samples were processed into paraffin blocks. Tissue sections were cut at a thickness of 5 using Kedee microtome (China JINHUA Kedi Co., Ltd, Zhejiang, China), then deparaffinized in toluene and rehydrated from graded of alcohols to distilled water. Endogenous peroxidase activity was blocked by treating the sections with 0.6% hydrogen peroxide in methanol for 20 min, followed by antigen retrieval treatment. (Dako, Glostrup, Denmark). The sections were then incubated with peroxidase blocking solution (Dako, Glostrup, Denmark) for 1 h at 37° C. to remove any nonspecific antigens. The specimens were incubated overnight at 4° C. with primary rabbit polyclonal antibody against Snail (1:200), Id1 (1:250) (Abcam, Cambridge, UK), cleaved caspase-3 and cleaved PARP (1:50; Cell Signalling Technology, Inc., Beverly, Mass., USA) and mouse monoclonal antibody against proliferating cell nuclear antigen (PCNA), Ki-67, E-cadherin (1:50; Santa Cruz Biotechnology, Santa Cruz, Calif., USA). After several rinse in TBS, the sections were incubated with Dako REALT EnVisionT/HRP, Rabbit/Mouse solution for 1 h at 37° C. The reaction was visualized by Dako REALT DAB⁺ chromogen. Mayer's haematoxylin (Dako, Glostrup, Denmark) was used as counter stain. Standard inverted light microscopy (Nikon, Tokyo, Japan) was used to analyze the slides.

4.1.10 Bioluminescence Imaging—In vivo bioluminescence imaging of luciferase activity from the spontaneous prostate tumor model was performed using IVIS imaging system (Xenogen Corp., Hopkinton, Mass., USA) with the LivingImage acquisition and analysis software (Xenogen Corp., Hopkinton, Mass., USA). D-Luciferin was dissolved to a concentration of 15 mg/ml in DPBS, filter-sterilized, and stored at −20° C. At the end of the treatment, mice were given i.p. injection of luciferin solution (150 mg/kg of body weight). Images were acquired 5 min after luciferin administration. Signal intensity was quantified as the sum of all detected photon counts with the region of interest from the tumors.

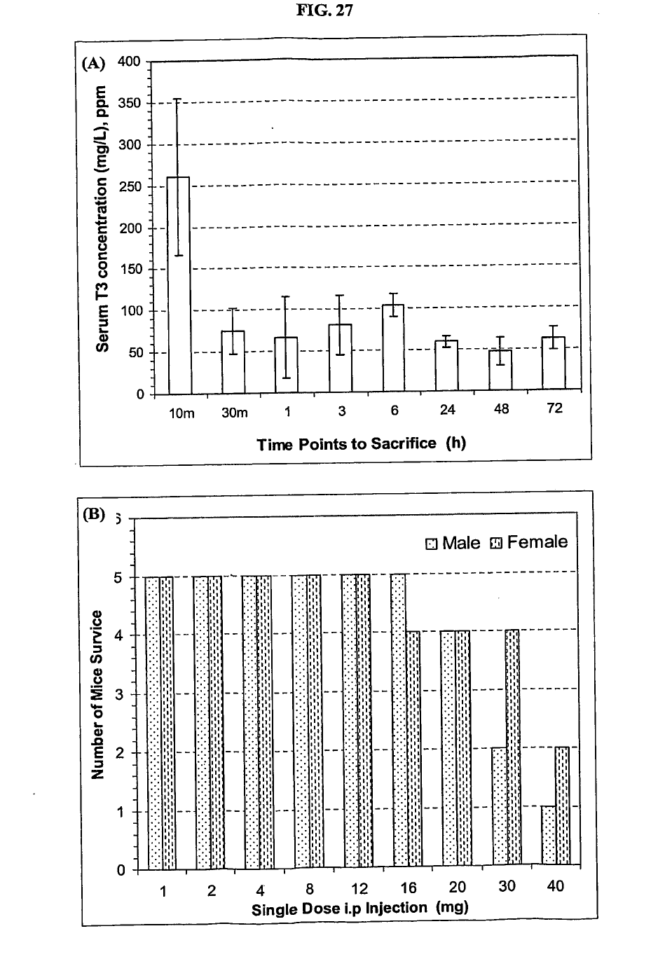

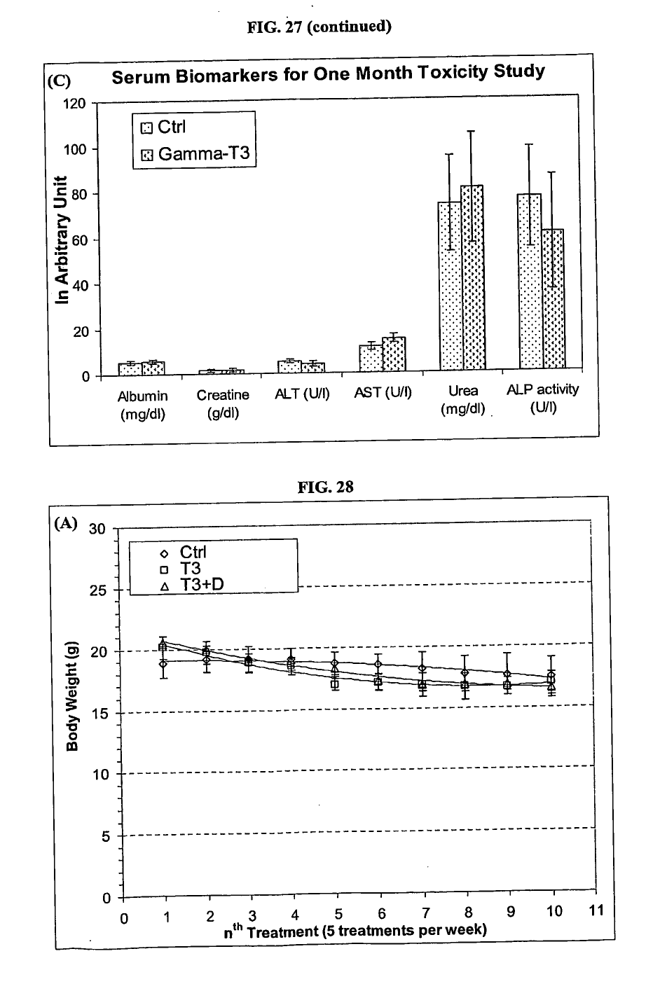

4.2 Results—Pharmacokinetics and single acute toxicity—Because γ-T3 inhibited proliferation and induced apoptosis in PCa cells in vitro reported herein, the antitumor effects of γ-T3 on PCa growth in vivo was investigated. It was started by studying the pharmacokinetic behaviour of γ-T3 in plasma after intra-peritoneal administration. Mice were injected with 1 mg of γ-T3 and blood was assayed for γ-T3 concentration at different time points thereafter. As shown in the serum pharmacokinetic profile (FIG. 27A), plasma γ-T3 level decreased from 260 ppm to 50 ppm within 30 min after administration. The level remains constant for at least 72 hours.

To evaluate single acute toxicity of γ-T3, γ-T3 was injected intraperitoneally (i.p.) at 9 escalating doses for the determination of maximum tolerated dose (MTD). The MTD is defined as the dose at which none of the 10 mice dies within 30-day observation period and at least one of the mice die in the next higher dose. As shown in FIG. 27B, MTD was found to be 12 mg. For mice receiving 5 dose i.p injections per week containing 1 mg of γ-tocotrienol or DMSO blank, there were no toxicological changes in any of the parameters examined (FIG. 27C).

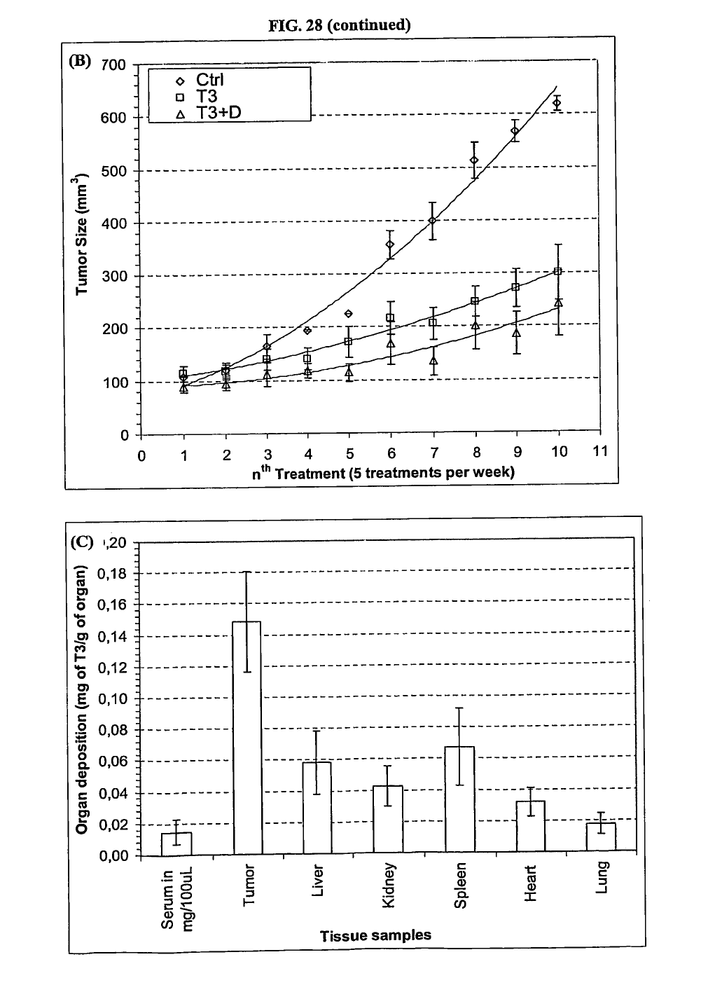

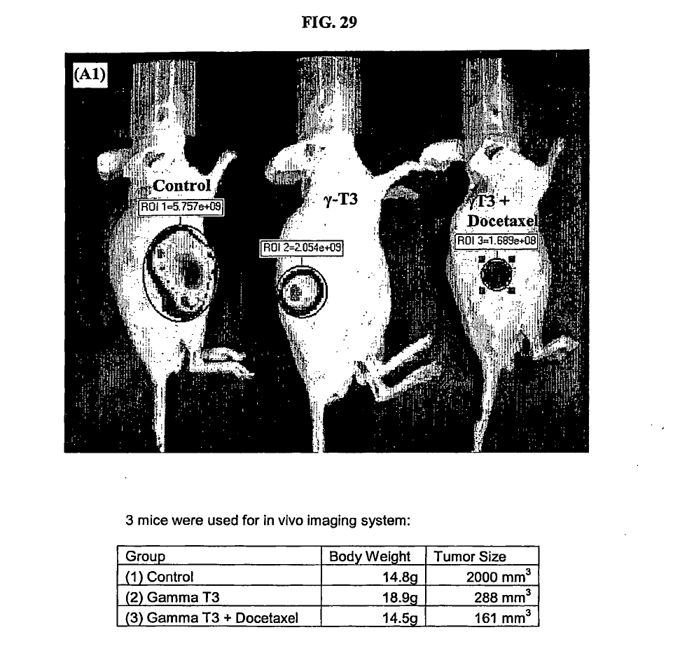

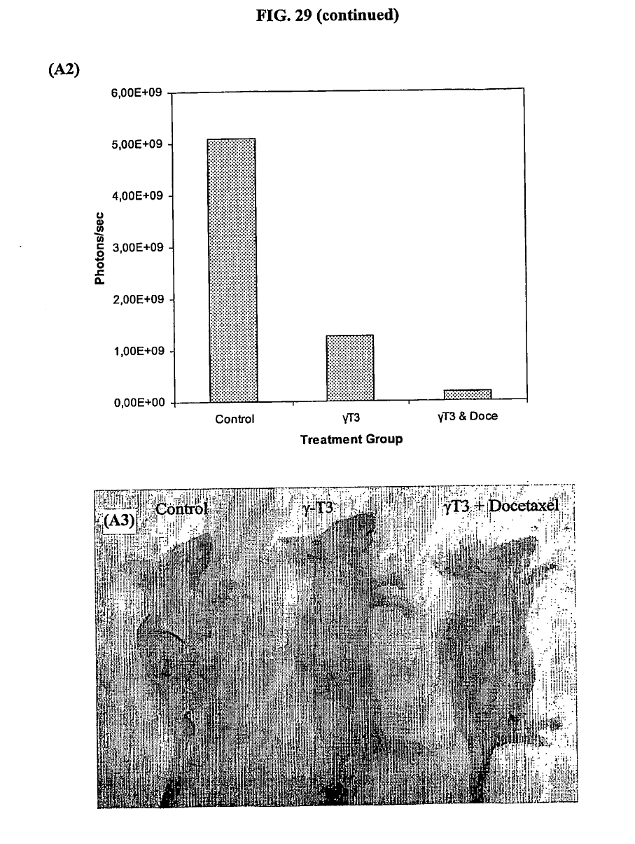

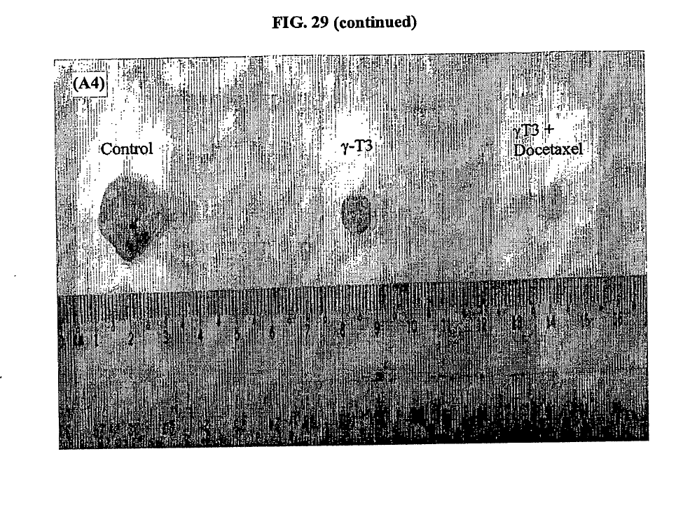

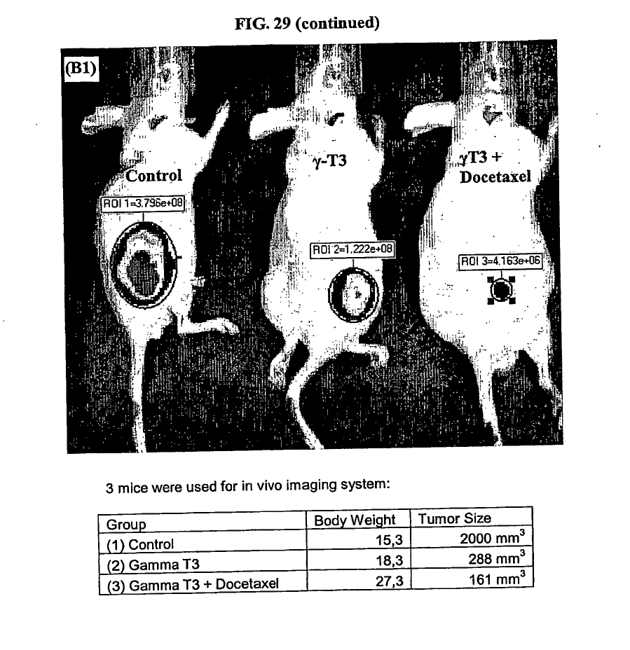

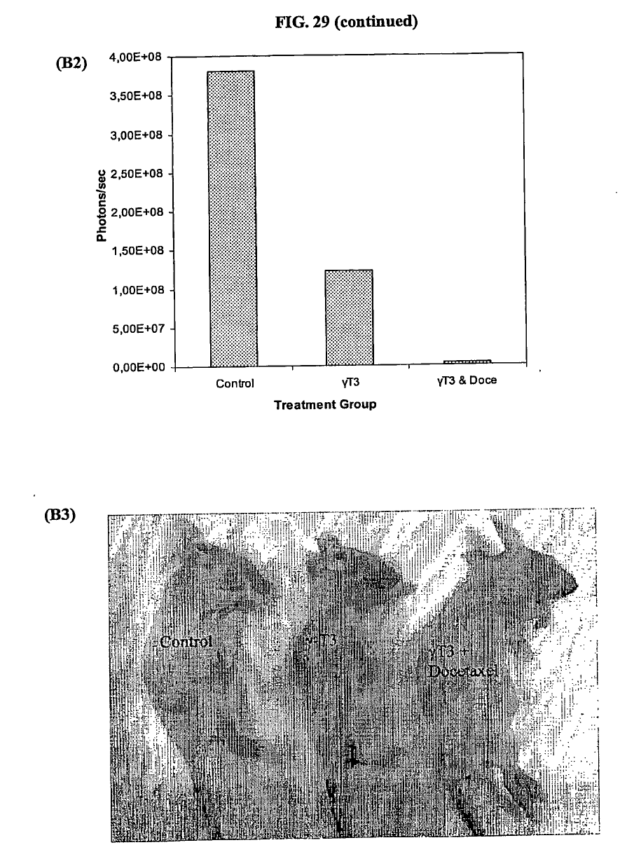

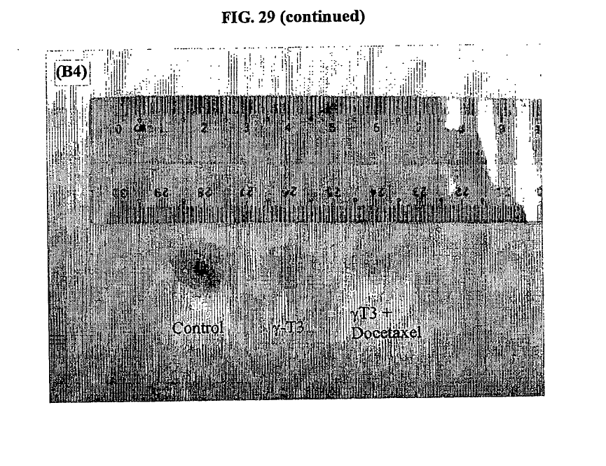

γ-T3 inhibits the growth of the PC-3-Luc prostate cancer xenograft—Because γ-T3 inhibited proliferation and induced apoptosis in PCa cells in vitro, the antitumor effects of γ-T3 on PCa growth in vivo was investigated. Athymic nude mice were allografted with PC-3-Luc cells and were divided into control (DMSO), γ-T3 and combined (γ-T3 plus Docetaxel) treatment groups. Dosage for γ-T3 (50 mg/kg/day) was selected because it provided a significant antitumor effect in the nude mice without inducing the treatment-related mortality observed with higher doses (FIG. 28A). Similarly for Doxetaxel, the dosage was determined to be 7.5 mg/kg/week. Tumor growth was monitored 5 times a week. There was no significant change in body weight throughout the entire study for all groups (FIG. 28A). Tumors in the control groups grew rapidly, reaching an average volume of 620±10 mm³ by day 14^{th} after the start of treatment. In contrast, tumor growth on mice that were administered with γ-T3 or γ-T3 plus Docetaxel was profoundly inhibited; with tumor volume remaining at an average of 300±48 mm³ and 240±62 mm³ respectively (FIG. 28B and FIG. 29). These results indicated that γ-T3 had a significant inhibitory effect on PCa growth in vivo (p value=0.0018) (FIG. 29).

Since serum γ-T3 level drops rapidly after administration (FIG. 27A). It is critical to understand if this is due to drug clearance or specific deposition to internal organs. At first, γ-T3 level of each of the vital organs from mice that treated with 50 mg/kg/day γ-T3 for 10 days with HPLC analysis was determined. As shown in FIG. 28C, spleen and liver was found to have the highest level of γ-T3 deposition at the end of the treatment period, although γ-T3 was also detectable in heart, kidney and lung tissues. More importantly, examination of the tumor tissues revealed that γ-T3 accumulated primarily within the tumors, reaching a concentration of 0.15±0.03 mg of γ-T3 per gram of wet weight (FIG. 28C) which was at least two-fold the amount detected in other internal organs. These results suggest that γ-T3 selectively deposits in prostate tumor tissues, which helps to explain why γ-T3 can exert significant anti-tumor activity at dosage that associate with no observable toxicity.

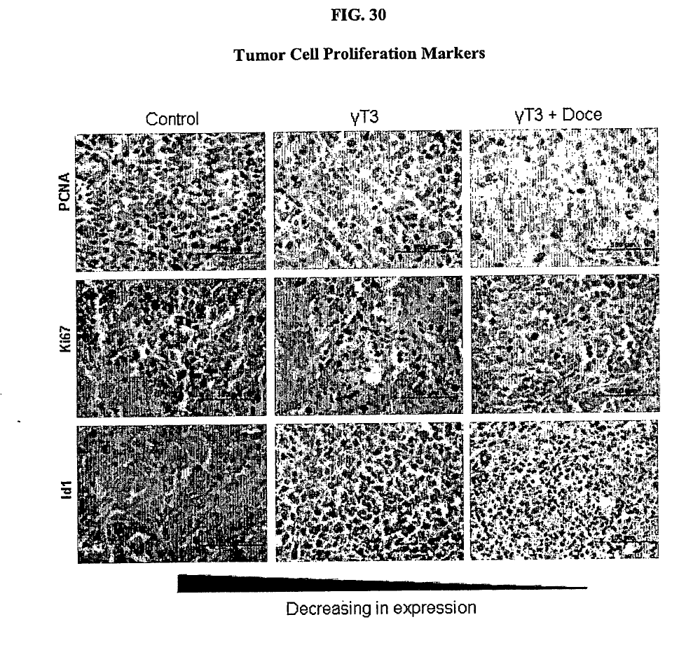

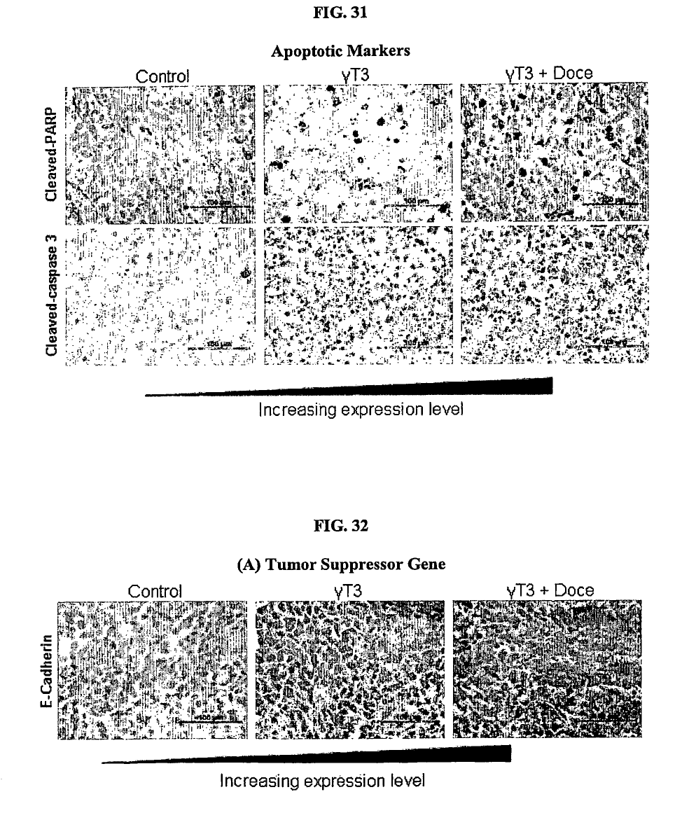

In vivo effect of γ-T3 on cancer cell proliferation and apoptosis—To confirm whether the anti-tumor effect of γ-T3 is, as described in the in vitro experiments (see item 2 above), mediated through inhibition of cell proliferation and induction of apoptosis, tumor tissues of the mice from each treatment group were examined by immunohistochemistry. As shown in FIG. 30, the antiproliferative effects of γ-T3 on PCa tumors were confirmed by examination of the level of PCNA, Ki67 and Id-1, which showed that all proteins were downregulated after treatment with γ-T3 or with γ-T3 plus Docetaxel. Meanwhile, γ-T3 also induced the level of cleaved caspase 3 and PARP (FIG. 31), suggesting that more cells underwent apoptosis after γ-T3 treatment.

γ-T3 antitumor effect on tumor suppressor gene—Down-regulation of E-cadherin expression is one of the most frequently reported characteristics of metastatic cancers.

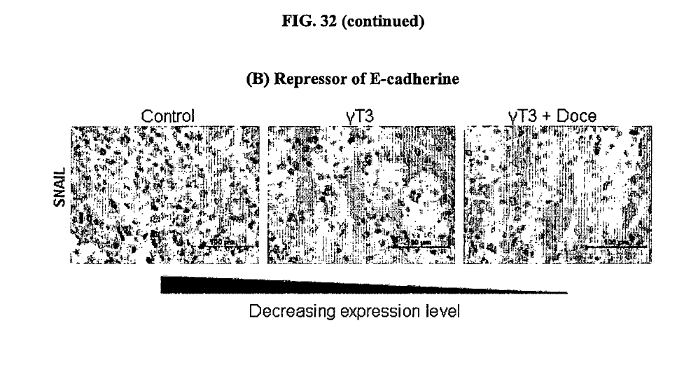

Restoration of E-cadherin expression in cancer cells leads to suppression of metastatic ability. In PCa, down-regulation of E-cadherin expression is correlated with high-grade tumors and poor prognosis, indicating their roles in PCa progression. Since γ-T3 was found to inhibit the in vitro invasion ability of prostate cancer cell through upregulation of E-cadherin expression, it was then analyzed if γ-T3 can also affect E-cadherin level in prostate cancer cells in vivo. E-cadherin expression of the tumor sections from the control-, γ-T3- and combined γ-T3-Docetaxel-treated groups of athymic nude mice was examined by immunohistochemistry and the results showed that E-cadherin was up-regulated after γ-T3 (FIG. 32A), whereas the repressor of E-cadherin, Snail, was down-regulated (FIG. 32B). These data suggested that in addition to inhibition of tumor growth; γ-T3 may possess in vivo anti-metastatic activity.

4.3 The experiments referred to in this section demonstrated that γ-T3 suppressed the growth of prostate tumor in nude mice, which is the first report on the in vivo anti-tumor effect of γ-T3 against prostate cancer. Study of γ-T3 antitumor effect in vivo are limited because of the lack of highly purified γ-T3 and the difficulties in delivering γ-T3 to tumor cells. It was shown that the inhibitory effect of γ-T3 on PCa cell growth is specific for the fast proliferating cells in vitro. Herein, it was observed that the intraperitoneal route of γ-T3 administration was effective in inhibiting PCa tumor growth.

The accumulation of γ-T3 is critical for the antitumor activities. It was found that γ-T3 was accumulated selectively in solid tumors, possibly due to high proliferation rate at the tumor tissue. It was further shown that γ-T3 was found in most of the vital organ. However, the γ-T3 deposition at the five vital organs (heart, liver, spleen, lungs, kidneys) was approximately half of that found in the solid tumor (FIG. 28C). The discrepancy on the findings is likely due to the method of administration, since γ-T3 was administered by intra-peritoneal injection in the experiments referred to herein, but was given to the mice by oral feeding in their study. Nevertheless, despite the deposition of γ-T3 in the vital organs, it has no observable effect on body weight, normal-organ weight and serum toxicity levels.

The mechanism by which γ-T3 inhibits tumor growth in vivo is poorly understood. The hydroxyl moiety, found in all tocochromanol molecules which mediate vitamin E's classical antioxidant properties, is generally believed to be unrelated to γ-T3's antitumor activities.

As described herein, it was found that γ-T3 upregulated E-cadherin gene that is thought to inhibit invasion, and metastasis. Also, Id-1, which is constitutively expressed by the PCa cell line PC-3, was repressed by γ-T3 treatment leading to the suppression of NFκB pathway molecules. Herein, it was possible to further confirm the anti-tumor activity of γ-T3 against prostate cancer under in vivo condition.

Because the cell proliferation and apoptosis are critical processes for tumor growth, the modulation of these processes by γ-T3 in our tumor model was investigated. Consistent with the significant antiproliferative effect of γ-T3 in vitro, it was observed a remarkable antiproliferative effect of γ-T3 in vivo, as evident by the repression of PCNA, Ki67 and Id-1 (FIG. 30) expression. Furthermore, a significant induction of apoptosis in PCa cells in vivo was observed. The exact mechanism responsible for γ-T3 induced apoptosis is not fully understood. The results of the experiments referred to herein support the process of apoptosis as an important mechanism of γ-T3 antitumor effect in vivo (FIG. 31). The implication of these observations is that γ-T3 may be used in synergy with other anti-proliferative agents against PCa.

Although tumor metastasis was not examined in the experiments referred to herein, it was found that γ-T3 treatment resulted in enhanced expression of E-cadherin and thus seems to support that γ-T3 may have anti-metastatic activity. Loss of E-cadherin function or expression has been implicated in cancer progression and metastasis because it decreases cellular adhesion within the tissue, resulting in an increase in cellular motility. This in turn may allow cancer cells to cross the basement membrane and invade surrounding tissues. The exact interaction with γ-T3 remains to be investigated but it may be a unique of tocotrienols in phospholipid membranes. Since it was also demonstrated herein that γ-T3 can inhibit the in vitro cancer cell invasion by induction of E-cadherin, the current finding provide strong evidence to warrant further investigation on the in vivo anti-metastatic effect of γ-T3.

In summary, it was demonstrated for the first time that γ-T3, a derivative of vitamin E, is capable of inhibiting PCa growth in vivo through inhibition of cancer cell proliferation and induction of apoptosis.

5. Evidences support that prostate cancer is originated from a rare sub-population of cells, namely prostate cancer stem cells (CSCs). Conventional therapies for prostate cancer are believed to target mainly the majority of differentiated tumor cells but spare CSCs, which may account for the subsequent disease relapse after the treatment. Therefore, successful elimination of CSCs may be an effective strategy to archive complete remission from this disease. It was demonstrated for the first time that γ-T3 can down-regulate the expression of prostate cancer stem cell markers (CD133/CD44) in androgen independent (AI) prostate cancer cell lines (PC-3 & DU145), as evident from Western blotting and flow cytometry analysis. Meanwhile, spheroid formation ability of the prostate cancer cells was significantly hampered by γ-T3 treatment. More importantly, pre-treatment of PC-3 cells with γ-T3 was found to interfere with the tumor initiation ability of the cells. The data referred to in this section suggest that γ-T3 can be an effective agent in targeting prostate CSCs.

5.1 Materials and Experimental Conditions

5.1.1 Prostate cancer cell lines PC-3, DU145 and bladder cancer cell line MGH-U1 (ATCC, Rockville, Md.) were maintained in RPMI 1640 medium (Invitrogen, Carlsbad, Calif.) supplemented with 1% (w/v) penicillin-streptomycin (Invitrogen, Carlsbad, Calif.) and 5% fetal bovine serum (Invitrogen, Carlsbad, Calif.). All cell types were kept at 37° C. in 5% CO₂ environment.

5.1.2 Tocotrienol isomers were extracted and purified as described above under item 1.1.2.

5.1.3 Generation of PC-3 cells stably expressing the luciferase protein—Luciferase-expressing PC-3 cell line, PC-3 luc, was generated using the Viralpower Lentiviral gene expression system (Invitrogen, Carlsbad, Calif.) according to the manufacturer's instruction. Briefly, HEK293 was transfected with the pLenti6-DEST-V5-Luc vector, which expresses the full length luciferase protein, together with the packaging mix provided with the Lentiviral expression system. Forty-eight hours after transfection, supernatant was collected, mixed with polybrene (8 μg/ml) and used to infect PC-3 cells. After infection, positive transfectants were selected as a pool by treatment with Blasticidine (10 μg/ml) for 6 days.

5.1.4 Cell viability assay—Cell viability upon γ-T3 treatment was measured by 3-(4,5-Dimethyl thiazol-2-yl)-2,5-diphenyl tetrazolium bromide (MTT) assay. Briefly, cells were seeded on 96 well-plates and treated with different concentrations of γ-T3 for the indicated time point. At the end of the treatment, MTT (Sigma, St. Louis, Mo.) was added into each well and incubated for 4 hrs at RT. DMSO was then added into each well to dissolve the formazan crystals. The plate was allowed to incubate for a further 5 min at RT and the optical density (OD) was measured at a wavelength of 570 nm on a Labsystem multiskan microplate reader (Merck Eurolab, Dietikon, Schweiz). All individual wells were set in triplicates. The percentage of cell viability was presented as OD ratio between the treated and untreated cells at indicated concentrations.

5.1.5 Western blotting was carried out as described above under 1.1.5. The membrane was incubated with primary antibodies directed against CD133 (Miltenyi Biotec, Auburn, Calif.), Bcl-2, PARP, cleaved caspase 3, 7, 9 (Cell Signaling, Technology Inc, Beverly, Mass.), CD44 and β-actin (Santa Cruz Biotechnology, Santa Cruz, Calif.). After washing with TBS-T, the membrane was incubated with secondary antibody against either mouse or rabbit IgG and the signals were visualized using ECL plus western blotting system (Amersham, Piscataway, N.J.).

5.1.6 Semi-quantitative RT-PCR—Total RNA was isolated using TRIZOL® reagent (Invitrogen, Carlsbad, Calif.) following the manufacturer's instruction. cDNA was synthesized by using SuperScript First-Strand Synthesis System for RT (Invitrogen, Carlsbad, Calif.) and PCR was carried out with GeneAmp® PCR System 9700 (Applied Biosystems, Foster City, Calif.). The primers sequence and PCR condition for RT-PCR of CD133 were previously described. The amounts of mRNA were quantified relative to GAPDH.

5.1.7 Spheroid formation assay—Spheroid formation assay was performed with a protocol modified from previous study (Folkins C, p3560). Briefly, cells were first trypsinized, washed with 1× PBS and resuspended in DMEM F12 medium. Two hundred cells were added into each well of a 24-well-plate pre-coated with polyHEMA (Sigma, St. Louis, Mo.). Cells will be grown in DMEM/F12 mem (Invitrogen, Carlsbad, Calif.) supplemented with 4 μg/mL insulin (Sigma, St. Louis, Mo.), B27 (Invitrogen, Carlsbad, Calif.), 20 ng/mL EGF (Sigma, St. Louis, Mo.), and 20 ng/mL basic FGF (Invitrogen, Carlsbad, Calif.). Fresh medium with the above supplements was added every day. γ-T3 was added at indicated time points and the number of spheroids was counted at day 14 of the assay or at the end of the treatment. Each experiment was repeated in triplicates and each data point represented the mean and standard derivation.

5.1.8 Flow cytometry—Flow cytometry analysis of the CD44 positive cells were performed with procedure known in the art. Briefly, cells were incubated in PBS containing 2% FBS with PE-conjugated anti-human CD44 antibody. Isotype-matched mouse immunoglobulins served as controls. Samples were then analyzed using a FACS Calibur flow cytometer and CellQuest software (BD Biosciences, San Jose, Calif., USA).

5.1.9 Orthotopic PC-3 xenograft model—The orthotopic model was established with procedures known in the art. Briefly, 8-week-old CB-17 SCID mice were anesthetized and placed under a dissecting microscope (Olympus, Tokyo, Japan). An incision at the midline of abdomen was made and the dorsal prostate was exposed at the base of the bladder. Equal amount of viable PC3-luc cells (2.6×10⁴ cells resuspended in 5 μl of serum free RPMI) with or without prior γ-T3 treatment for 24 hrs were injected into the dorsal prostates of the mice. Organs were replaced and the abdomen was closed. To detect the bioluminescent signal of the cells, mice were anesthetized and then injected with 80 mg/kg of D-luciferin solution by i.p. (Xenogen Corporation, Cranbury, N.J.). Signal was captured by Xenogen IVIS 100 series imaging system. Tumors progression was monitored by measuring the bioluminescent signal (units of photons per second per unit area) every 2 weeks until 6-week post tumor implantation. Mice were sacrificed by cervical dislocation and tumors were collected and fixed in 10% formalin. All surgical and animal handling procedures were carried out according to the guidelines of the Committee on the Use of Live Animals in Teaching and Research (CULATR), The University of Hong Kong.

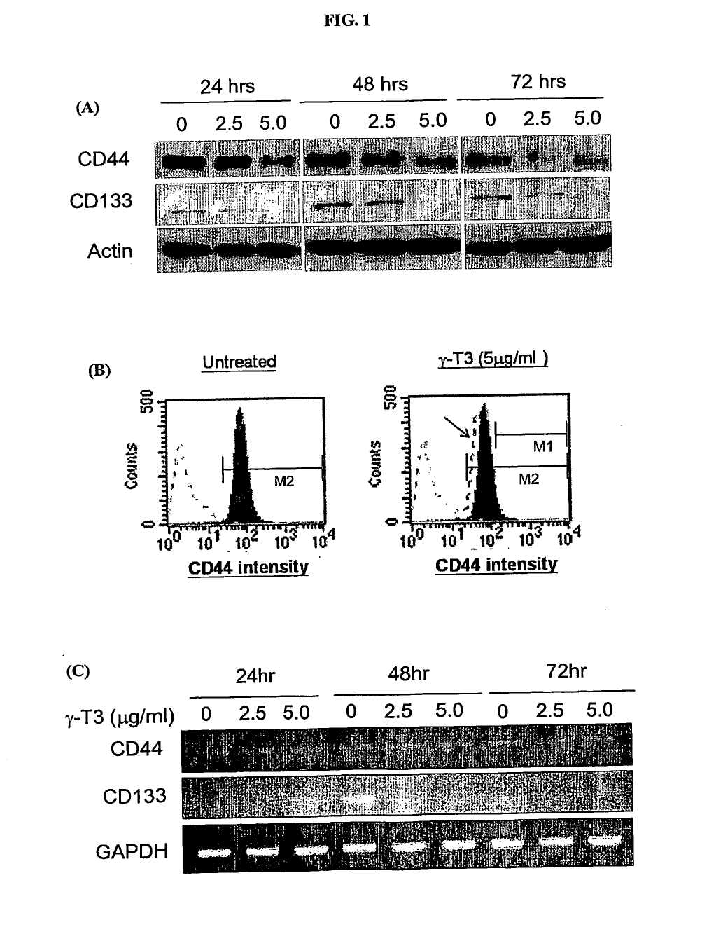

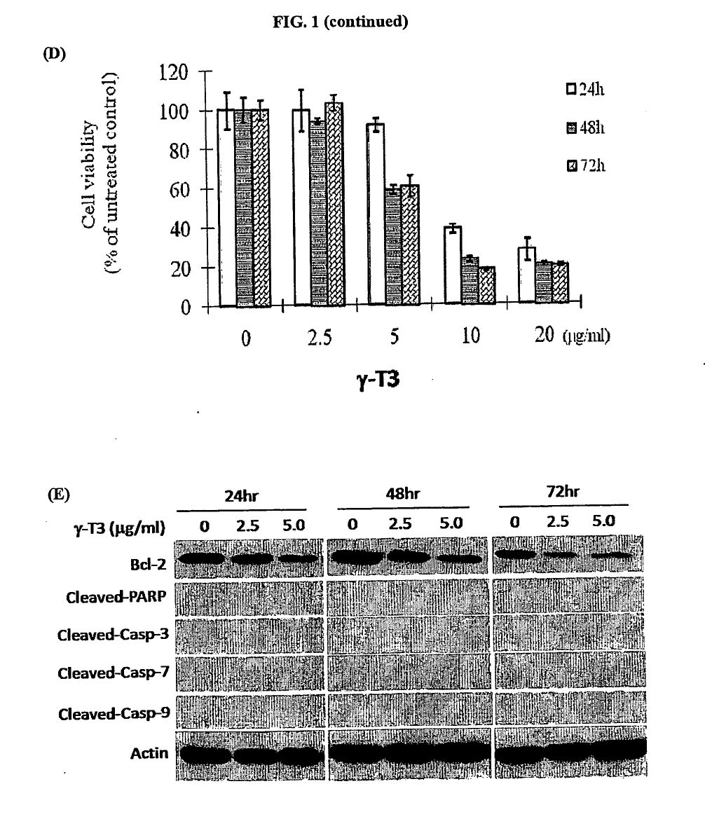

5.2 Results—Effect of γ-T3 on CSC markers expression—In order to test if γ-T3 affects CSC property, the effect of γ-T3 on the expression of prostate CSC markers in PC-3 cell line, which has been reported to contain the highest percentage of CSCs among other cell lines (PCa stem cell Oncogene) was investigated at first. PC-3 cells were first treated with increasing dose of γ-T3 (0, 2.5 & 5 μg/ml) for 24, 48 and 72 hours. After the treatment, the expression of the two established prostate CSC markers, CD44 and CD133, were examined by western blotting. As shown in FIG. 1A, protein expression of CD44 was significantly down regulated after γ-T3 treatment in a time and dose-dependent manner. Similar effect was also observed in CD133, suggesting that γ-T3 treatment is able to target the CSC population (FIG. 1A). To confirm if γ-T3 affect CSC marker expression, the change of CD44⁺ population in PC-3 cells after γ-T3 treatment by flow cytometry was examined. After 24 hrs of γ-T3 treatment (5 μg/ml), the population of CD44⁺ PC-3 cells was found to decrease comparing to untreated control (FIG. 1B), which is consistent with the western blotting results.

To test if the changes in CD44 and CD133 is due to decrease in gene transcription, mRNA levels of CD44 and CD133 in PC-3 cells that treated with 2.5 and 5 μg/ml of γ-T3 were evaluated by RT-PCR. As shown in FIG. 1C, decrease of CD133 mRNA was observed in cells that treated with 2.5 and 5 μg/ml of γ-T3 for 48 and 72 hrs. Downregulation of CD44 mRNA was also observed in cells that treated with γ-T3 for 72 hrs. These results indicated that γ-T3 can suppress CD44 and CD133 expression at the transcriptional level. Interestingly, the downregulation of the CSC marker expressions by γ-T3 are not the result of the induction of apoptosis, as viability assay (FIG. 1D) as well as western blotting of common apoptotic markers (FIG. 1E) both failed to detect a drastic induction of apoptosis.

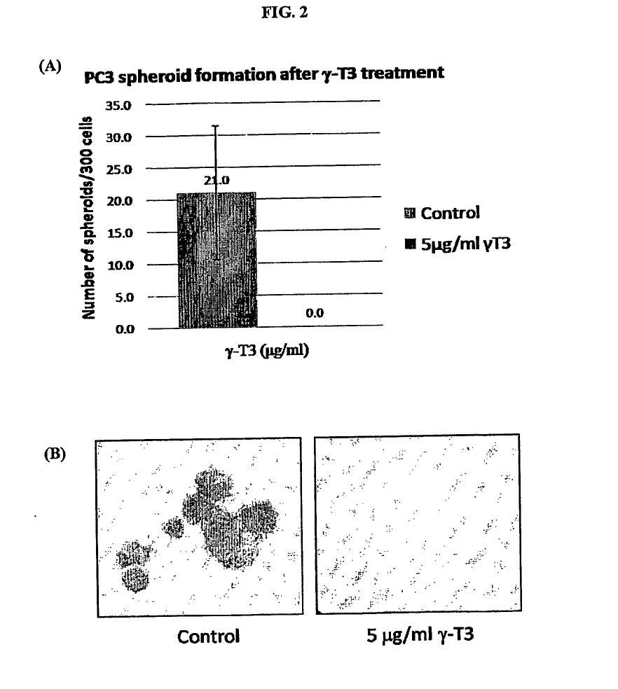

γ-T3 inhibits prostasphere formation of PC-3 under non-adherent culture condition—The ability to form prostaspheres in non-adherent culture is one of the characteristics of prostate cancer stem cells. To further examine the effect γ-T3 on prostate CSCs, prostasphere formation of PC-3 cells were studied in the presence or absence of γ-T3. This is done by plating PC-3 cells into a Poly-HEMA pre-coated plate, which prevents the cells from surface attachment. Cells were allowed to grow in serum replacement medium with or without the γ-T3 (5 μg/ml). As shown in FIG. 2A, after culturing the cells for 10 days, an average of 21 prostaspheres per well were found in the untreated group. However, no prostasphere can be observed in all wells that treated with γ-T3 (FIGS. 2A&B). These results indicate that γ-T3 can effectively inhibit prostasphere formation of the prostate cancer cells.

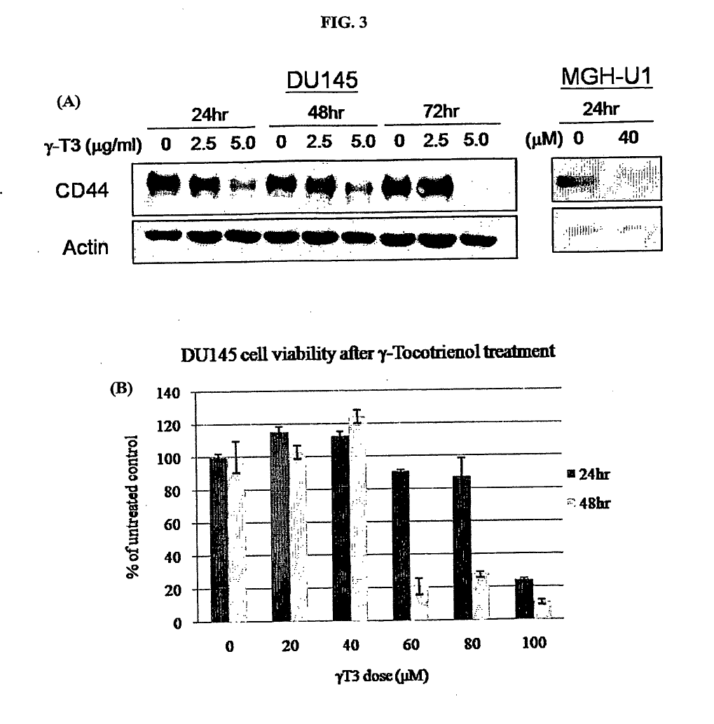

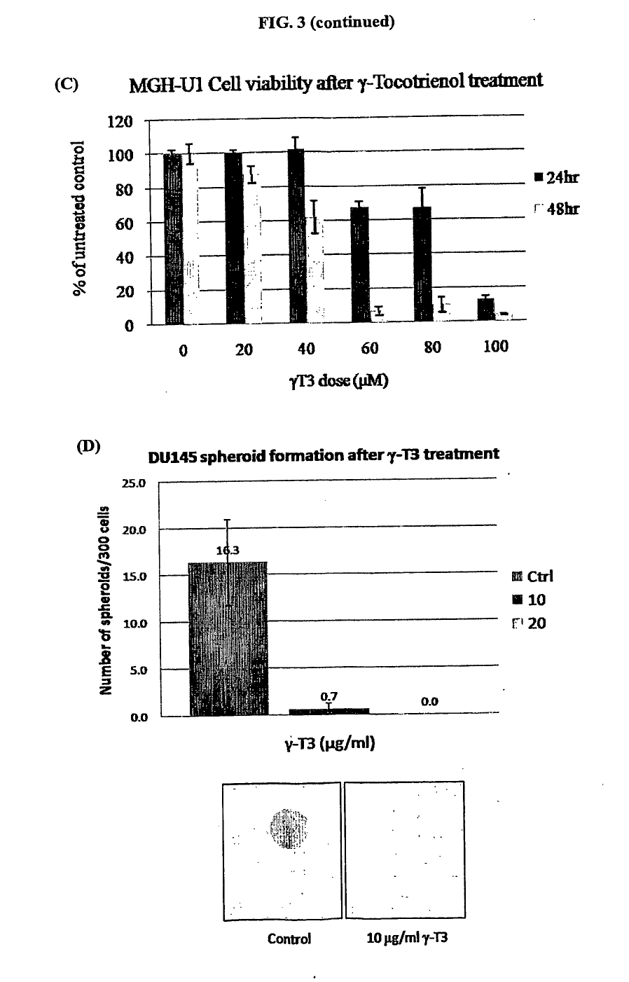

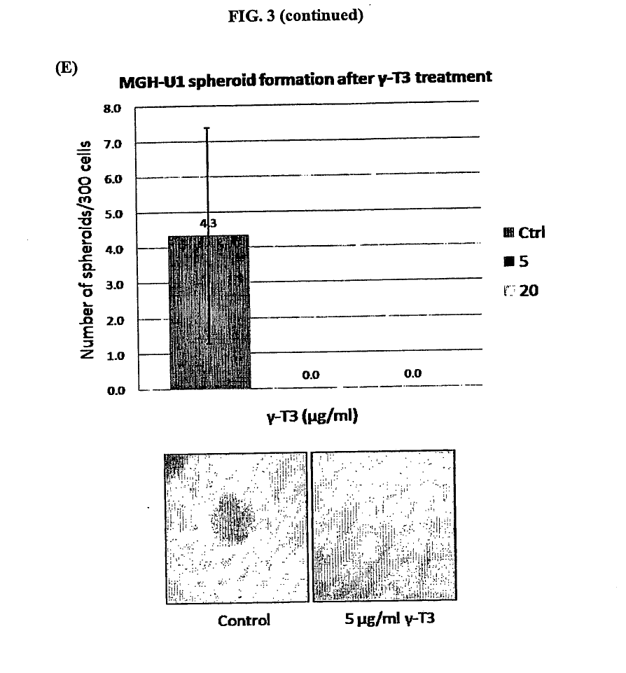

γ-T3 suppresses CSC property in other cancer cell lines—Results from the above experiments suggested that γ-T3 can target CD44⁺ CD133⁺ cancer stem-like cell in the androgen independent prostate cancer cell line PC-3. However, it is possible that the suppressive effect is only specific to PC-3 cells rather than a general effect. This prompts to repeat the experiments using other cancer cell lines. DU145 is another prostate cancer cell line which has been shown to possess CSC properties, and as shown in FIG. 3A, γ-T3 treatment at doses that have minimum effect on cell viability also results in suppression of CD44 expression in a time and dose dependent manner. Meanwhile, spheroid formation ability of DU145 was almost completely suppressed by γ-T3 treatment (FIG. 3D). Similar effect was also observed in a bladder cancer cell line (MGH-U1) (FIGS. 3B, C & E), suggesting that the observed effect of γ-T3 on CSCs is not restricted for prostate cancer.

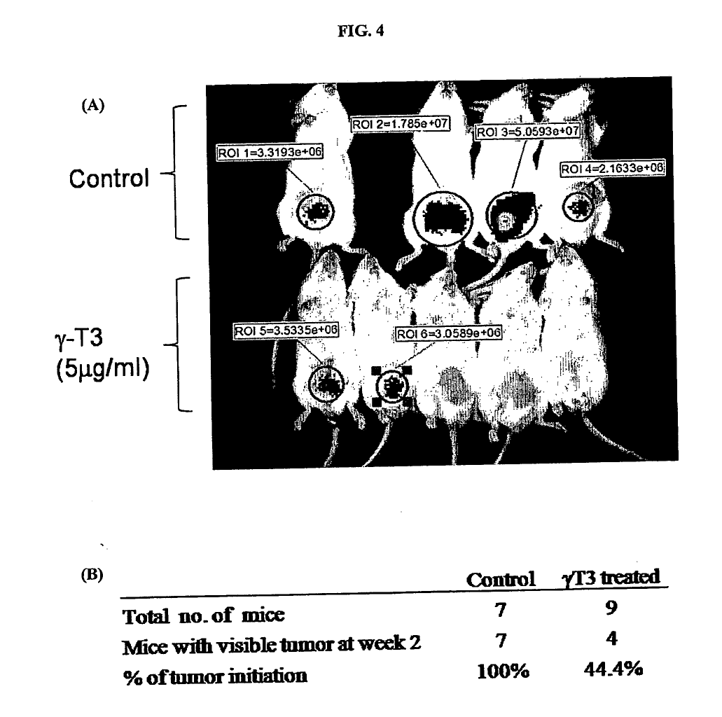

Gamma-T3 significantly reduces the tumorigenicity of prostate cancer cell in vivo—Since CSC is suggested to a play role in cancer initiation, it is possible that γ-T3 treatment may inhibit the tumor formation ability of PC-3 cells. To test this hypothesis, PC-3 cells constitutively expressing the luciferase reporter gene (PC-3-luc) were first pre-treated with 5 μg/ml of γ-T3 or vehicle for 24 hrs. Subsequently, equal number of viable PC-3-luc cells from treatment and control group were injected orthotopically into the SOD mice and the tumor formation was monitored by live bioluminescent imaging. As shown in FIG. 4, two weeks after implantation, all 7 mice implanted with vehicle treated PC-3-luc developed tumors. However, more than half of the mice (5 out of 7) implanted with γ-T3 pretreated PC-3-luc failed to develop visible tumor (FIG. 4). The significant decrease of tumor initiation rate indicates that γ-T3 can reduce the tumorigenic potential of highly aggressive PC-3 cells, which is likely due to the decrease of CSC population after γ-T3 treatment.

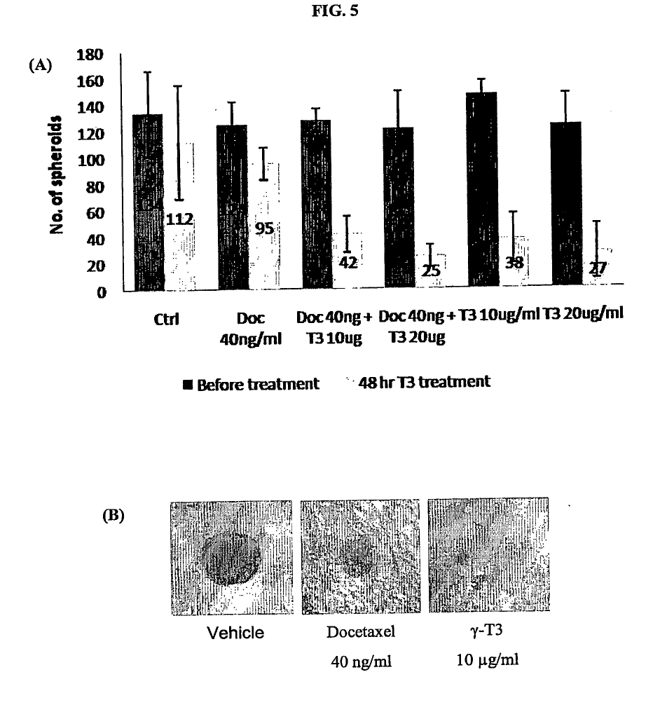

Gamma-T3 effectively eliminates chemo-resistant cancer stem-like cells—It was also tested whether γ-T3 can also target the pre-formed prostasphere, which has been shown to contain enriched-CSC population. The prostaspheres were formed by growing DU145 cells in non-adherent culture for 14 days, where each prostasphere reached a considerable size. As expected, these prostaspheres were highly resistant to chemotherapeutic agent, such as Docetaxel (FIG. 5A). At dosage of 40 ng/ml, which is known to induce apoptosis in DU145 cells, Docetaxel failed to induce any observable effect on prostasphere number, suggesting that the CSC-enriched cells are highly resistant to Docetaxel. However, decrease of spheroid number for 70% and 76% were observed when the spheroids were treated with 10 μg/ml and 20 μg/ml of γ-T3 (FIG. 5A). In addition to the decrease in spheroid number, γ-T3 treatment also reduced the size of the spheroid as well as changed the spheroid shape into a more diffuse structure (FIG. 5B).

5.3 It was demonstrated herein for the first time that γ-T3 has anti-CSC effect, as evidenced by the downregulation of CSC markers and the suppression of prostasphere and tumor formation by γ-T3. Putative cancer stem cell in the prostate was first identified in 2005, where they were found to express CD44+/alpha2beta1hi/CD133+ surface markers. These cancer-initiating cells have also been identified in established androgen dependent cell line LNCaP and androgen independent prostate cancer cell lines DU145.

In was demonstrated herein that CSC markers CD44 and CD133 expressed in PC-3 cells were both downregulated by γ-T3 treatment (FIG. 1). It was also observed a significant decrease of CD44 in androgen independent prostate cancer cell line DU145 and bladder cancer cell line MGH-U1 (FIG. 4). Interestingly, it was not possible to detect any significant decrease in cell viability or increase in cellular apoptosis (FIG. 1) after γ-T3 treatment, indicating that the dosages of γ-T3 that was used in this study is capable of targeting the CSC population, but is insufficient for inducing apoptosis of the non-CSC cells. This further implies that γ-T3 may have specific effect against CSC.

The ability to form spheres in non-adherent, serum free condition is a key property of stem cells. Recently, spheroid formation assay was used as a method to identify and to enrich the putative CSCs. In the experiments referred to herein, all the 3 malignant cell lines PC-3, DU145 and MGH-U1 were able to form spheroids in non-adherent culture, suggesting the presence of cancer stem-like cells within these cell lines. According to the results, 7%, 5.4% and 1.4% of cells from PC-3, DU145 and MGH-U1 respectively (FIGS. 3&4) were capable of forming spheroid. Since prostaspheres are enriched with CSC (6.25% and 12.2% of CD133⁺, CD44⁺ cells in PC-3 and DU145 spheres, respectively), the inhibitory effect of γ-T3 on prostasphere formation supports that γ-T3 can be a potent agent in targeting or eliminating prostate cancer stem-like cells in vitro (FIG. 5). A similar effect was also observed in MGH-U1 cells, where γ-T3 treatment resulted in 100% inhibition in spheroid formation (FIG. 3E). Although the putative cancer stem cells in bladder is yet to be identified, the suppressive effect of γ-T3 towards the stem cell property of MGH-U1 suggests that the anti-CSC effect of γ-T3 does not restricted to prostate cancer. This is support by the finding that γ-T3 can also downregulate CD44 expression in bladder cancer cells.

The ability of the CSCs to generate serial transplantable tumor in vivo suggests that they are likely to be the tumor initiating cells (TIC). This hypothesis is supported by the fact that the isolated CSC population is more tumorigenic than the non-CSC counterpart when injected into the immuno-compromised mice. As disclosed herein, when PC-3 cells were pre-treated with γ-T3, a sharp decrease in tumorigenic potential was observed (FIG. 4). Despite the fact that all γ-T3 pretreated PC-3 can eventually develop detectable tumors (data not shown), the drastic decrease in detectable tumor at early tumor initiation stage and the delay of tumor formation support our hypothesis that γ-T3 is potent in targeting the prostate CSCs.

The presence of CSC is suggested to contribute to chemo-resistance. Prostate cancer cells are in general highly resistant to common chemotherapeutic agents. Docetaxel represent the only effective chemodrug which has demonstrated significant improvement in patient survival. The IC90 dosage of Docetaxel for DU145 is 1.01 ng/ml. However, in this study, treatment of the prostasphere with 40 ng/ml Docetaxel was unable to induce significant reduction of prostasphere numbers, further confirming that CSC is resistant to chemodrug treatment. γ-T3, on the other hand, was able to induce a dramatic decrease in prostasphere number, which is associated with the dissociation of prostaspheres (FIG. 5). This evidence strongly suggests that the anti-CSC effect is likely to account for the chemosensitizing effect of γ-T3. In summary, it was demonstrated for the first time that γ-T3 treatment not only downregulates prostate CSC marker expressions, but also effectively inhibits the CSC properties.

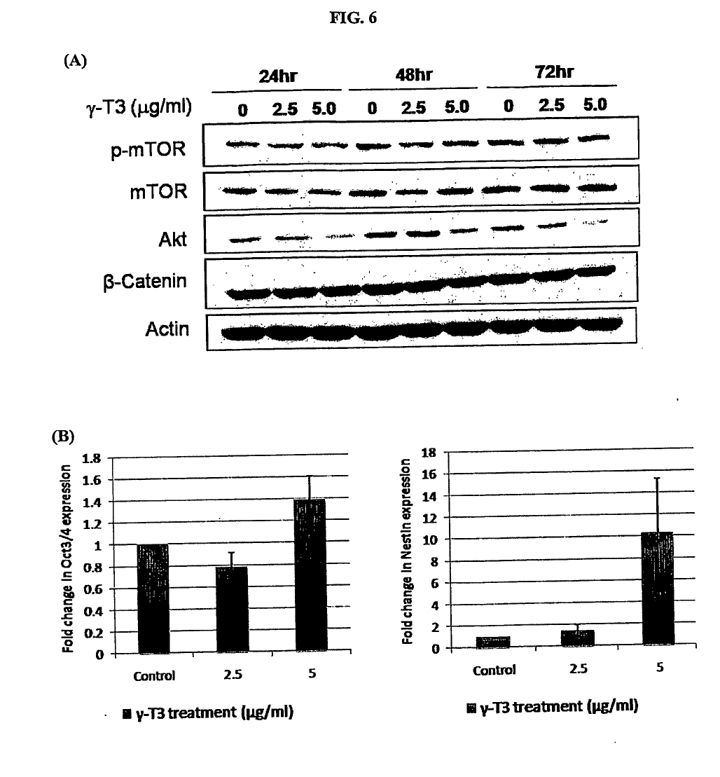

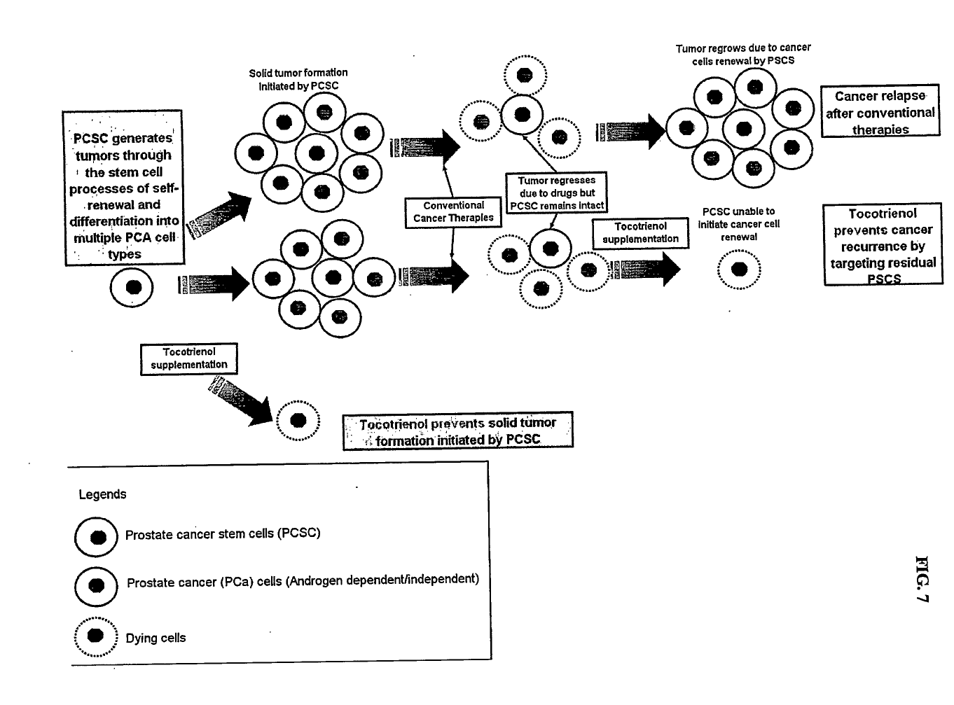

The results illustrated in FIG. 6(A) show that the expression of AKT is downregulated using a low dose (i.e. between 0 to about 5 μg/ml or about 2.5 μg/ml or about 5 μg/ml) of gamma-tocotrienol, suggesting de-activation of AKT signalling pathway. Previously, lentivirus-mediated expression of constitutively active AKT in dissociated prostate cells results in the regeneration of prostate tubules containing prostate intraepithelial neoplasia lesions that progress to frank carcinoma.

The results illustrated in FIG. 6(B) show that the expression of Oct3/4 and Nestin is upregulated using a low dose (i.e. between 0 to about 5 μg/ml or about 2.5 μg/ml or about 5 μg/ml) of gamma-tocotrienol, suggesting activation of stem-cell phenotypes (gain in pluripotency). In general, those two genes are closely regulated because too much or too little will actually cause differentiation of the cells.

6. Prevention of Formation of Prostate Intraepithelial Neoplasia (PIN)

For experiments, 5-week old prostate cancer mouse models previously published (Gabril, M. Y., Duan, W., et al., Molecular Therapy (2005), vol. 11, no. 3, p. 348; Greenberg et al., Proc Natl Acad Sci USA (1995), vol. 92, pp. 3439-3443; Duan, W., Gabril, M. Y., et al., Oncogene (2005) 24, 1510-1524; Wang S, Gao J, et al., Cancer Cell., 2003, vol. 4, no. 3, pp. 209-21; Gabril, M. Y., Onita, T., et al., Gene Ther., 2002, vol. 9, no. 23, pp. 1589-99) are used. All animal experiments were conducted according to standard protocols approved by Animal Care Committee. Genotyping was performed in PSP-TGMAP, and KIMAP mice were identified by a quick PCR genotyping protocol as previously reported (see above references referred to under item 6).

In one exemplary experiment, the animals are given 1 mg of gamma tocotrienol per day for 30 weeks via oral gavage. At the end of 10, 20 and 30 weeks, animals are sacrificed. The prostate along with the male accessory glands, i.e., the ventral and dorsolateral prostate lobes, seminal vesicles, and coagulation gland, were dissected out separately for histopathological characterization of prostate tumor development, prostate intraepithelial neoplasia (PIN) development and microinvasion.

Protocols for IHC with an ABC kit (StreptABC Complex Kit; DAKO, Mississauga, ON, Canada) were preformed following the standard Chromogranin (polyclonal antibody, from Dia Sorin, Stillwater, Minn., USA) was used at 1:500 dilutions.

To study tumor development, some modifications were adopted according to the established diagnostic criteria previously reported (see above references referred to under item 6). According to heterogeneity and multifocality of the clinical standard for CaP diagnosis, a close-to human genetically engineered (GE) mouse standard system for histological grading and scoring was established in this study. The architectural patterns of adenocarcinoma observed were assessed by five different GE histological grades: GE-Grade 1 (very well differentiated), single, separate, uniform glands closely packed, with definite boundaries; GE-Grade 2 (well differentiated), single, separate uniform glands loosely packed, with irregular edges; GE-Grade 3 (glands with variable and distorted architecture), single, separate, uniform scattered glands and smoothly circumscribed papillary/cribriform masses; GE-Grade 4 (poorly differentiated), cribriform masses with ragged, invading edges and fused glands; GE-Grade 5, nonglandular solid, rounded masses of cells, cribriform architecture with foci of central necrosis (known as comedocarcinoma) and undifferentiated anaplastic carcinomas. Based on the most prevalent GE histological grade (the “primary pattern/Grade”) and the second most prevalent GE histological pattern (“secondary pattern/grade”), the new GE scoring system was derived by adding the primary pattern GE grade number to the secondary GE grade number. If only one pattern was seen throughout, the score was derived by the doubling grade number. As illustrated in FIG. 8, mice fed with a composition comprising gamme or delta tocotrienol or a mixture of gamma and delta tocotrienol did not develop PIN.

1. A method of preventing cancer or preventing the recurrence of cancer after undergoing a cancer treatment by administering a composition comprising at least one of γ-tocotrienol or δ-tocotrienol, wherein the cancer is selected from the group consisting of melanoma, prostate cancer, prostate intraepithelial neoplasia, colon cancer, liver cancer, bladder cancer, breast cancer and lung cancer.

2. The method of claim 1, wherein the cancer treatment is selected from the group consisting of surgery, radiation therapy, chemotherapy, hormonal therapy, immunotherapy, differentiating agents, and combinations of the aforementioned treatments or therapies.

3. The method of claim 1 or 2, wherein the cancer is prostate cancer.

4. The method of any one of the preceding claims, wherein the composition is a γ-tocotrienol and/or δ-tocotrienol enriched formulation.

5. The method of any one of the preceding claims, wherein the composition comprises more than 10 wt. % of γ-tocotrienol or δ-tocotrienol or a mixture of γ-tocotrienol and δ-tocotrienol based on the total wt. % of the composition.

6. The method of any one of the preceding claims, wherein the composition comprises γ-tocotrienol and δ-tocotrienol in a ratio of 1:Y wherein Y is less than 10.

7. The method of any one of the preceding claims, wherein the composition further comprises α-tocotrienol and/or β-tocotrienol and/or α-tocopherol.

8. The method of any one of the preceding claims, wherein the composition is administered in an amount of between about 10 mg and about 1000 mg total tocotrienol wt. % content per 60-kg adult or between about 10 mg and about 500 mg total tocotrienol wt. % content per 60-kg adult.

9. The method of any one of the preceding claims, wherein the composition is administered in an amount to obtain a serum level concentration in blood of an animal between about 0.1 to 30 mg/L or between about 10 to 30 mg/L for each individual tocotrienol isomer.

10. The method of claim 9, wherein the animal is a mammal.

11. The method of claim 10, wherein the mammal is selected from the group consisting of human, pig, horse, mouse, rat, cow, dog and cat.

12. The method of any one of the preceding claims, wherein the composition is formulated as liquid native oil, a water soluble emulsion, a cold water dispersible powder, and beadlets.

13. The method of any one of the preceding claims, wherein the composition is administered as tablet, or gel, or dragée, or sustained-release formulation, or ointment, or injectable formulation or in encapsulated form.

14. The method of any one of the preceding claims, wherein the composition is administered oral, or intradermal, or subcutaneous or intraperitoneal.

15. The method of any one of the preceding claims, wherein the composition further comprises a substance selected from the group consisting of a green tea polyphenol, a organosulfur compound, a protein-bound polysaccharide isolated from Trametes versicolor or Coriolus versicolor, a red carotenoid pigment and combinations of the aforementioned substances.

16. The method of claim 15, wherein the green tea polyphenol is selected from the group consisting of epicatechin (EC), epigallocatechin (EGC), epicatechin gallate (ECG) and epigallocatechin gallate (EGCG).

17. The method of claim 15, wherein the organosulfur compound is selected from the group consisting of S-allylmercaptocysteine derived from garlic and allicin derived from garlic.

18. The method of claim 15, wherein the protein-bound polysaccharide is polysaccharide-K (Krestin, PSK) or the polysaccharide peptide (PSP).

19. The method of claim 15, wherein the red carotenoid pigment is lycopene.

20. A composition comprising at least one of γ-tocotrienol or δ-tocotrienol and (2R,3S)-N-carboxy-3-phenylisoserine, N-tert-butylester, 13-ester with 5,20-epoxy-1,2,4,7,10,13-hexahydroxytax-11-en-9-one-4-acetate-2-benzoate, trihydrate (Docetaxel) and/or (5Z)-5-(dimethylaminohydrazinylidene)imidazole-4-carboxamide (Dacarbazine).

21. A method of inhibiting or reversing of cancer by administering a composition comprising at least one of γ-tocotrienol or δ-tocotrienol together with (2R,3S)—N-carboxy-N-tert-butylester-3-phenylisoserine, 13-ester with 5,20-epoxy-1,2,4,7,10,13-hexahydroxytax-11-en-9-one-4-acetate-2-benzoate, trihydrate (Docetaxel) and/or (5Z)-5-(dimethylaminohydrazinylidene)imidazole-4-carboxamide (Dacarbazine).

22. The method of claim 21, wherein the cancer is selected from the group consisting of melanoma, prostate cancer, colon cancer, liver cancer, prostate intraepithelial neoplasia, bladder cancer, breast cancer and lung cancer.

23. The method of claim 21 for inhibiting or reversing of melanoma, wherein the composition comprises at least one of γ-tocotrienol or δ-tocotrienol together with (2R,3S)—N-carboxy-N-tert-butylester-3-phenylisoserine, 13-ester with 5,20-epoxy-1,2,4,7,10,13-hexahydroxytax-11-en-9-one-4-acetate-2-benzoate, trihydrate (Docetaxel) and/or (5Z)-5-(dimethylaminohydrazinylidene)imidazole-4-carboxamide (Dacarbazine).

24. The method of claim 21 for inhibiting or reversing of prostate cancer, wherein the composition comprises at least one of γ-tocotrienol or δ-tocotrienol together with (2R,3S)—N-carboxy-N-tert-butylester-3-phenylisoserine, 13-ester with 5,20-epoxy-1,2,4,7,10,13-hexahydroxytax-11-en-9-one-4-acetate-2-benzoate, trihydrate (Docetaxel).

25. The method of claim 21 for inhibiting or reversing of breast cancer, wherein the composition comprises at least one of γ-tocotrienol or δ-tocotrienol together with (2R,3S)—N-carboxy-N-tert-butylester-3-phenylisoserine, 13-ester with 5,20-epoxy-1,2,4,7,10,13-hexahydroxytax-11-en-9-one-4-acetate-2-benzoate, trihydrate (Docetaxel).

26. The composition of claim 20 or the method of any one of claims 21 to 25, wherein the composition is a γ-tocotrienol and/or δ-tocotrienol enriched formulation.

27. The composition of claim 20 or 26 or the method of any one of claims 21 to 26, wherein the composition comprises more than 10% of γ-tocotrienol or δ-tocotrienol or a mixture of γ-tocotrienol and δ-tocotrienol based on the total weight of the composition.

28. The composition of claims 20 or 26 to 27 or the method of any one of claims 21 to 27, wherein the composition comprises γ-tocotrienol and δ-tocotrienol in a ratio of 1:Y wherein Y is less than 10.

29. The composition of claims 20 or 26 to 28 or the method of any one of claims 21 to 28, wherein the composition further comprises α-tocotrienol and/or β-tocotrienol and/or α-tocopherol.

30. The composition of claims 20 or 26 to 29 or the method of any one of claims 21 to 29, wherein the composition further comprises a substance selected from the group consisting of a green tea polyphenol, a organosulfur compound, a protein-bound polysaccharide, a polysaccharide peptide isolated from Trametes versicolor or Coriolus versicolor, a red carotenoid pigment and combinations of the aforementioned substances.

31. The method of any one of claims 21 to 30, wherein the composition is administered in an amount of between about 10 mg and about 1000 mg total tocotrienol wt. % content per 60-kg adult or between about 10 mg and about 500 mg total tocotrienol wt. % content per 60-kg adult.

32. The method of any one of claims 21 to 31, wherein the composition is administered in an amount to obtain a serum level concentration in blood of an animal between about 0.1 to 30 mg/L or between about 10 to 30 mg/L for each individual tocotrienol isomer.

33. The method of claim 32, wherein the animal is a mammal.

34. The method of claim 33, wherein the mammal is selected from the group consisting of human, pig, horse, mouse, rat, cow, dog and cat.

35. The method of any one of claims 21 to 34, wherein the composition is administered in a water solubilized form.

36. The method of any one of claims 21 to 35, wherein the composition is administered as tablet, or gel, or dragée, or sustained-release formulation, or ointment, or injectable formulation or in encapsulated form.

37. The method of any one of claims 21 to 35, wherein the composition is administered intradermal, or subcutan or intraperitoneal or oral.

38. Use of a composition comprising at least one of γ-tocotrienol or δ-tocotrienol for the manufacture of a medicament for preventing cancer in an animal body or for preventing the recurrence of cancer in an animal body after undergoing a cancer treatment, wherein the cancer is selected from the group consisting of melanoma, prostate cancer, colon cancer, liver cancer, prostate intraepithelial neoplasia, bladder cancer, breast cancer and lung cancer.

39. Use of a composition comprising at least one of γ-tocotrienol or δ-tocotrienol together with (2R,3S)—N-carboxy-3-phenylisoserine, N-tert-butylester, 13-ester with 5,20-epoxy-1,2,4,7,10,13-hexahydroxytax-11-en-9-one-4-acetate-2-benzoate, trihydrate (Docetaxel) or (5Z)-5-(dimethylaminohydrazinylidene)imidazole-4-carboxamide (Dacarbazine) for the manufacture of a medicament for the treatment of cancer.

40. A method of manufacturing a composition according to claim 20 comprising mixing at least one γ-tocotrienol or δ-tocotrienol with (2R,3S)—N-carboxy-3-phenylisoserine, N-tert-butylester, 13-ester with 5,20-epoxy-1,2,4,7,10,13-hexahydroxytax-11-en-9-one-4-acetate-2-benzoate, trihydrate (Docetaxel) and/or (5Z)-5-(dimethylaminohydrazinylidene)imidazole-4-carboxamide (Dacarbazine).
